(19) United States
(12) Patent Application Publication
(10) Pub. No.: US 2022/0339246 A1
(43) Pub. Date: Oct. 27, 2022

(54) COMPOSITIONS INCLUDING MOLECULES OF MODIFIED MRNA AND METHODS OF USING THE SAME

(71) Applicant: ICAHN SCHOOL OF MEDICINE AT MOUNT SINAI, New York, NY (US)

(72) Inventors: Lior ZANGI, New York, NY (US); Keerat KAUR, New York, NY (US)

(21) Appl. No.: 17/753,660

(22) PCT Filed: Sep. 11, 2020
(86) PCT No.: PCT/US2020/050411

§ 371 Date: Mar. 10, 2022

Related U.S. Application Data
(60) Provisional application No. 62/898,958, filed on Sep. 11, 2019.

(51) Int. Cl. A61K 38/17; A61P 9/04; A61K 48/00; A61P 9/10; C12N 5/077

(52) U.S. Cl. CPC A61K 38/1709; A61P 9/04; A61K 48/005; A61P 9/10; C12N 5/0657; C12N 2501/998

(57) ABSTRACT

The present disclosure relates to compositions including molecules of modified mRNA encoding GATA Binding Protein 4, modRNA encoding Myocyte Enhancer Factor 2C, modRNA encoding T-box 5, modRNA encoding Heart- and neural crest derivatives-expressed protein 2, modRNA encoding dominant negative transforming growth factor beta, and modRNA encoding domimant negative Wingless-related integration site 8a, wherein said molecules of modRNAs are present in said composition in a ratio. The present disclosure further relates to pharmaceutical compositions, methods for increasing a ratio of a number of cardiomyocytes to a number of non-cardiomyocytes within a population of cells, methods for treating cardiac injury, methods for stimulating vascular regeneration, methods for treating of stroke, and methods for enhancing wound healing.

This application claims benefit of U.S. Provisional Patent Application Ser. No. 62/898,958, filed on Sep. 11, 2019, which is hereby incorporated by reference in its entirety.

This invention was made with government support under HL142768-01 and RO1 HL149137-01 awarded by the National Institutes of Health. The government has certain rights in the invention.

### SEQUENCE LISTING

The present application contains a Sequence Listing, created on Sep. 11, 2020; the file, in ASCII format, is designated as 3710049AWO_sequencelisting_ST25.txt and is 116 kilobytes in size. The file is hereby incorporated by reference in its entirety into the instant application.

### FIELD

The present disclosure relates generally to compositions including molecules of modified mRNA and methods of using the same.

### BACKGROUND

Ischemic heart disease remains a major cause of morbidity and mortality in the western world causing a significant societal and economic burden. A problem is a massive loss of cardiomyocytes (CMs) following a myocardial infarction (MI), with a corresponding increase in cardiac fibroblasts and development of scar tissue. An ability to increase, promote, restore, or otherwise stimulate the number or growth of cardiac myocytes would present a highly advantageous improvement to treatments for recovery to MI and other cardiac insults where low numbers or loss of cardiomyocytes impair cardiac function, health, or repair. A possibility includes modifying activity of cellular factors involved in development of cardiomyocytes from precursors or from other cell types and promote cardiomyocyte health and/or proliferation. However, difficulties in identifying and modifying activity of, and a lack of understanding of, relevant cellular factors and signaling pathways hampers such goals. Modifying targeted pathways, at an intracellular locus, with treatments that regulate expression of genetically encoded products is difficult due to uncontrolled and low efficiency of the delivered genes. Furthermore, a lack of understanding of what cellular factor or factors to target, and in what relative amounts, in order to obtain desired increases in cardiomyocytes hampers such goals.

The adult mammalian heart has a very limited regeneration capacity; therefore, upon ischemic injury, large numbers of CMs die and are replaced by non-contracting, collagen-rich cardiac scar tissue that builds for several weeks post injury in a process called remodeling. Directly reprogramming the scar cells (i.e. non-CMs) into functional CMs is one strategy/approach now being evaluated by researchers as a way to overcome this lack of CMs and the addition of non-CMs in the left ventricle (LV) post ischemic injury.

In 2010, Idea et al. identified three (Gata4, Mef2C and Tbx5 (GMT)) out of 14 transcription factors that can reprogram cardiac or tail-tip fibroblasts into CM-like cells. Ieda et al., “Direct Reprogramming of Fibroblasts Into Functional Cardiomyocytes by Defined Factors,” Cell 142:375-86 (2010). Since then, a large number of publications have established that GMT may induce cardiac reprogramming. Abad et al., “Notch Inhibition Enhances Cardiac Reprogramming by Increasing MEF2C Transcriptional Activity,” Stem Cell Reports 8:548-560 (2017); Christoforou et al., “Transcription Factors MYOCD, SRF, Mesp1 and SMARCD3 Enhance the Cardio-Inducing Effect of GATA4, TBX5, and MEF2C During Direct Cellular Reprogramming,” PLoS One 8:e63577 (2013); Fu et al., “Direct Reprogramming of Human Fibroblasts Toward a Cardiomyocyte-Like State,” Stem Cell Reports 1:235-47 (2013); Hirai et al., “Accelerated Direct Reprogramming of Fibroblasts Into Cardiomyocyte-like Cells With the MyoD Transactivation Domain,” Cardiovasc Res. 100:105-13 (2013); Ifkovits et al., “Inhibition of TGFβ Signaling Increases Direct Conversion of Fibroblasts to Induced Cardiomyocytes,” PLoS One. 9:e89678 (2014); Mohamed et al., “Chemical Enhancement of In Vitro and In Vivo Direct Cardiac Reprogramming,” Circulation 135:978-995 (2017); Muraoka et al., “MiR-133 Promotes Cardiac Reprogramming by Directly Repressing Snail and Silencing Fibroblast Signatures,” EMBO J. 33:1565-81 (2014); Nam et al., “Induction of Diverse Cardiac Cell Types by Reprogramming Fibroblasts With Cardiac Transcription Factors,” Development 141:4267-78 (2014); Nam et al. “Reprogramming of Human Fibroblasts Toward a Cardiac Fate,” Proc. Natl. Acad. Sci. USA 110:5588-93 (2013); Qian et al., “In Vivo Reprogramming of Murine Cardiac Fibroblasts Into Induced Cardiomyocytes,” Nature 485:593-8 (2012); Singh et al., “MiR-590 Promotes Transdifferentiation of Porcine and Human Fibroblasts Toward a Cardiomyocyte-Like Fate by Directly Repressing Specificity Protein 1,” J. Am. Heart Assoc. 5 (2016); Song et al., “Heart Repair by Reprogramming Non-Myocytes With Cardiac Transcription Factors,” Nature 485:599-604 (2012); Wada et al., “Induction of Human Cardiomyocyte-Like Cells From Fibroblasts by Defined Factors,” Proc. Natl. Acad. Sci. USA. 110:12667-72 (2013); Yamakawa et al., “Fibroblast Growth Factors and Vascular Endothelial Growth Factor Promote Cardiac Reprogramming under Defined Conditions,” Stem Cell Reports 5:1128-1142 (2015); and Zhao et al., “High-Efficiency Reprogramming of Fibroblasts Into Cardiomyocytes Requires Suppression of Pro-Fibrotic Signalling,” Nat. Commun. 6:8243 (2015). Several research groups have shown that for human cardiac reprogramming, adding Myocardin (Myocd) to GMT is essential for successful reprogramming. Christoforou et al., “Transcription Factors MYOCD, SRF, Mesp1 and SMARCD3 Enhance the Cardio-Inducing Effect of GATA4, TBX5, and MEF2C During Direct Cellular Reprogramming,” PLoS One 8:e63577 (2013); Fu et al., “Direct Reprogramming of Human Fibroblasts Toward a Cardiomyocyte-Like State,” Stem Cell Reports 1:235-47 (2013); Mohamed et al., “Chemical Enhancement of In Vitro and In Vivo Direct Cardiac Reprogramming,” Circulation 135:978-995 (2017); Muraoka et al., “MiR-133 Promotes Cardiac Reprogramming by Directly Repressing Snail and Silencing Fibroblast Signatures,” EMBO J. 33:1565-81 (2014); Nam et al., “Induction of Diverse Cardiac Cell Types by Reprogramming Fibroblasts With Cardiac Transcription Factors,” Development 141:4267-78 (2014); Wada et al., “Induction of Human Cardiomyocyte-Like Cells From Fibroblasts by Defined Factors,” Proc. Natl. Acad. Sci. USA. 110:12667-72 (2013); and Addis et al., “Optimization of Direct Fibroblast Reprogramming to Cardiomyocytes Using Calcium Activity as a Functional Measure of Success,” J. Mol. Cell Cardiol. 60:97-106 (2013). Moreover, other studies demonstrated that including basic helix-loop-helix transcription factor Hand2 to GMT (GMTH) leads to superior reprogramming efficiency. Abad et al., “Notch Inhibition Enhances Cardiac Reprogramming by Increasing MEF2C Transcriptional Activity,” Stem Cell Reports 8:548-560 (2017); Hirai et al., “Accelerated Direct Reprogramming of Fibroblasts Into Cardiomyocyte-like Cells With the MyoD Transactivation Domain,” Cardiovasc Res. 100:105-13 (2013); Ifkovits et al., “Inhibition of TGFβ Signaling Increases Direct Conversion of Fibroblasts to Induced Cardiomyocytes,” PLoS One. 9:e89678 (2014); Nam et al., “Induction of Diverse Cardiac Cell Types by Reprogramming Fibroblasts With Cardiac Transcription Factors,” Development 141:4267-78 (2014); Nam et al. “Reprogramming of Human Fibroblasts Toward a Cardiac Fate,” Proc. Natl. Acad. Sci. USA 110:5588-93 (2013); Song et al., “Heart Repair by Reprogramming Non-Myocytes With Cardiac Transcription Factors,” Nature 485:599-604 (2012); Yamakawa et al., “Fibroblast Growth Factors and Vascular Endothelial Growth Factor Promote Cardiac Reprogramming under Defined Conditions,” Stem Cell Reports 5:1128-1142 (2015); and Zhao et al., “High-Efficiency Reprogramming of Fibroblasts Into Cardiomyocytes Requires Suppression of Pro-Fibrotic Signalling,” Nat. Commun. 6:8243 (2015).

To date, there are two major obstacles in cardiac reprogramming: one is the poor efficiency of GMT and GMTH and the other is the use of viral transfection (mostly retro- or lentiviruses) and small molecules that can lead to detrimental side effects and regulatory safety concerns. The initial study of CM reprogramming with GMT indicated 4.8% reprogramming efficiency (cTnT⁺ cells) in vitro (Ieda et al., “Direct Reprogramming of Fibroblasts Into Functional Cardiomyocytes by Defined Factors,” Cell 142:375-86 (2010)) and 12% conversion into CM-like cells (α-Myosin Heavy Chain (αMHC)⁺ cells) in vivo as shown by a lineage-tracing mouse MI model. Qian et al., “In Vivo Reprogramming of Murine Cardiac Fibroblasts Into Induced Cardiomyocytes,” Nature 485:593-8 (2012). Further, the addition of Hand2 to GMT together with Notch inhibitor and AKT-kinase augmented reprogramming efficiency to 70% in mouse embryonic fibroblasts (MEFs) (Abad et al., “Notch Inhibition Enhances Cardiac Reprogramming by Increasing MEF2C Transcriptional Activity,” Stem Cell Reports 8:548-560 (2017)) while combination of GMTH with several different microRNAs (miRs) and small molecules lead to about 60% reprogramming efficiency. Zhao et al., “High-Efficiency Reprogramming of Fibroblasts Into Cardiomyocytes Requires Suppression of Pro-Fibrotic Signalling,” Nat. Commun. 6:8243 (2015). In vivo reprogramming with GMT or GMTH showed 12% or 6.5% conversion of non-CMs to CM-like cells (Qian et al., “In Vivo Reprogramming of Murine Cardiac Fibroblasts Into Induced Cardiomyocytes,” Nature 485:593-8 (2012) and Song et al., “Heart Repair by Reprogramming Non-Myocytes With Cardiac Transcription Factors,” Nature 485:599-604 (2012)), though in both cases this moderate efficiency did improve MI outcome. Qian et al., “In Vivo Reprogramming of Murine Cardiac Fibroblasts Into Induced Cardiomyocytes,” Nature 485:593-8 (2012) and Song et al., “Heart Repair by Reprogramming Non-Myocytes With Cardiac Transcription Factors,” Nature 485:599-604 (2012). To date, however, reprogramming studies have used a lenti- or retro-viral delivery system to deliver reprogramming genes to non-CMs. Ieda et al., “Direct Reprogramming of Fibroblasts Into Functional Cardiomyocytes by Defined Factors,” Cell 142:375-86 (2010); Abad et al., “Notch Inhibition Enhances Cardiac Reprogramming by Increasing MEF2C Transcriptional Activity,” Stem Cell Reports 8:548-560 (2017); Christoforou et al., “Transcription Factors MYOCD, SRF, Mesp1 and SMARCD3 Enhance the Cardio-Inducing Effect of GATA4, TBX5, and MEF2C During Direct Cellular Reprogramming,” PLoS One 8:e63577 (2013); Fu et al., “Direct Reprogramming of Human Fibroblasts Toward a Cardiomyocyte-Like State,” Stem Cell Reports 1:235-47 (2013); Hirai et al., “Accelerated Direct Reprogramming of Fibroblasts Into Cardiomyocyte-like Cells With the MyoD Transactivation Domain,” Cardiovasc Res. 100:105-13 (2013); Ifkovits et al., “Inhibition of TGFβ Signaling Increases Direct Conversion of Fibroblasts to Induced Cardiomyocytes,” PLoS One. 9:e89678 (2014); Mohamed et al., “Chemical Enhancement of In Vitro and In Vivo Direct Cardiac Reprogramming,” Circulation 135:978-995 (2017); Muraoka et al., “MiR-133 Promotes Cardiac Reprogramming by Directly Repressing Snail and Silencing Fibroblast Signatures,” EMBO J. 33:1565-81 (2014); Nam et al., “Induction of Diverse Cardiac Cell Types by Reprogramming Fibroblasts With Cardiac Transcription Factors,” Development 141:4267-78 (2014); Nam et al. “Reprogramming of Human Fibroblasts Toward a Cardiac Fate,” Proc. Natl. Acad. Sci. USA 110:5588-93 (2013); Qian et al., “In Vivo Reprogramming of Murine Cardiac Fibroblasts Into Induced Cardiomyocytes,” Nature 485:593-8 (2012); Singh et al., “MiR-590 Promotes Transdifferentiation of Porcine and Human Fibroblasts Toward a Cardiomyocyte-Like Fate by Directly Repressing Specificity Protein 1,” J. Am. Heart Assoc. 5 (2016); Song et al., “Heart Repair by Reprogramming Non-Myocytes With Cardiac Transcription Factors,” Nature 485:599-604 (2012); Wada et al., “Induction of Human Cardiomyocyte-Like Cells From Fibroblasts by Defined Factors,” Proc. Natl. Acad. Sci. USA. 110:12667-72 (2013); Yamakawa et al., “Fibroblast Growth Factors and Vascular Endothelial Growth Factor Promote Cardiac Reprogramming under Defined Conditions,” Stem Cell Reports 5:1128-1142 (2015); Zhao et al., “High-Efficiency Reprogramming of Fibroblasts Into Cardiomyocytes Requires Suppression of Pro-Fibrotic Signalling,” Nat. Commun. 6:8243 (2015); and Addis et al., “Optimization of Direct Fibroblast Reprogramming to Cardiomyocytes Using Calcium Activity as a Functional Measure of Success,” J. Mol. Cell Cardiol. 60:97-106 (2013). The fundamental problems with using these viral gene delivery approaches into the heart are myocardial inflammation and the potential for insertional mutagenesis. Wasala et al., “The Evolution of Heart Gene Delivery Vectors,” J. Gene Med. 13:557-65 (2011). Furthermore, current methods cause a long-term increase in cardiac development genes, which are transiently expressed under normal conditions and thus may generate untoward effects over longer periods. Gata4 overexpression can induce cardiac hypertrophy (Liang et al., “The Transcription Factors GATA4 and GATA6 Regulate Cardiomyocyte Hypertrophy In Vitro and In Vivo,” J. Biol. Chem. 276:30245-53 (2001)), and increased Hand2 expression is associated with heart defects (Tamura et al. “Overdosage of Hand2 Causes Limb and Heart Defects in the Human Chromosomal Disorder Partial Trisomy Distal 4q,” Hum. Mol. Genet. 22:2471-81 (2013)). Thus, a transient, non-immunogenic gene delivery reprogramming method, without compromising the genomic integrity have advantages over current methodologies.

Modified mRNA (modRNA) is a safe, non-immunogenic, transient gene delivery method that has no risk of genome integration. This group and others have used modRNA to deliver genes into the heart post injury. Carlsson et al., “Biocompatible, Purified VEGF-A mRNA Improves Cardiac Function after Intracardiac Injection 1 Week Post-myocardial Infarction in Swine,” Mol. Ther. Methods Clin. Dev. 9:330-346 (2018); Magadum et al., “Ablation of a Single N-Glycosylation Site in Human FSTL 1 Induces Cardiomyocyte Proliferation and Cardiac Regeneration,” Mol. Ther. Nucleic Acids 13:133-143 (2018); Zangi et al., “Modified mRNA Directs the Fate of Heart Progenitor Cells and Induces Vascular Regeneration After Myocardial Infarction,” Nat. Biotechnol. 31:898-907 (2013); and Zangi et al., “Insulin-Like Growth Factor 1 Receptor-Dependent Pathway Drives Epicardial Adipose Tissue Formation After Myocardial Injury,” Circulation 135:59-72 (2017). The pharmacokinetics of modRNA in the heart allow gene expression to jumpstart promptly after delivery and persist for up to 10 days. Sultana et al., “Optimizing Cardiac Delivery of Modified mRNA,” Mol. Ther. 25:1306-1315 (2017). modRNA has been used directly in vitro to reprogram human fibroblasts into hepatocytes and mesenchymal stem cells into neural-like cells (Kim et al., “Single-Factor SOX2 Mediates Direct Neural Reprogramming of Human Mesenchymal Stem Cells via Transfection of In Vitro Transcribed mRNA,” Cell Transplant. 27:1154-1167 (2018) and Simeonov et al., “Direct Reprogramming of Human Fibroblasts to Hepatocyte-Like Cells by Synthetic Modified mRNAs,” PLoS One. 9:e100134 (2014)), however, no studies have yet reported using modRNA for direct reprogramming in vivo.

The present disclosure is directed to overcoming these and other deficiencies in the art.

### SUMMARY

A first aspect relates to a composition including molecules of modified mRNA (modRNA) encoding GATA Binding Protein 4 (G), modRNA encoding Myocyte Enhancer Factor 2C (M), modRNA encoding T-box 5 (T), modRNA encoding Heart- and neural crest derivatives-expressed protein 2 (H), modRNA encoding dominant negative transforming growth factor beta (dnT), and modRNA encoding dominant negative Wingless-related integration site 8a (dnW), wherein said molecules of modRNAs are present in said composition in a ratio of G:M:T:H:dnT:dnW.

In an example, said ratio is 1:1:1:1:1:1. In another example, said ratio is 2:2:2:2:0.7:0.7. In another example, said ratio is 2:1:1:1:0.7:0.7. In another example, said ratio is 1:2:1:1:0.7:0.7. In another example, said ratio is 1:1:2:1:0.7:0.7. In another example, said ratio is 1:1:1:2:0.7:0.7. In another example, said ratio is 1:2:1:2:0.5:0.5. In one example, when the composition comprises a ratio of G:M:T:H:dnT:dnW, M is present in an amount that is higher than other modRNA present in said composition. In one example, when the composition comprises a ratio of G:M:T:H:dnT:dnW, H is present in an amount that is higher than other modRNA present in said composition.

Another aspect relates to a pharmaceutical composition including a foregoing composition or example thereof and a pharmaceutically acceptable carrier.

Another aspect relates to a method for increasing a ratio of a number of cardiomyocytes to a number of non-cardiomyocytes within a population of cells including contacting said population of cells with the foregoing composition or example thereof. In an example, said non-cardiomyocytes include cardiac fibroblasts.

Another aspect relates to a method for treating cardiac injury including administering to a patient in need of such treatment a therapeutically effective amount of the foregoing composition or example thereof. In an example, the cardiac injury includes myocardial infarction. In another example, the cardiac injury includes reperfusion injury.

Another aspect relates to a method for stimulating vascular regeneration following ischemic damage including contacting tissue damaged by ischemic damage with the foregoing composition or example thereof.

Another aspect relates to a method for treating of stroke including administering to a patient in need of such treatment a therapeutically effective amount of the foregoing composition or example thereof.

Another aspect relates to a method for enhancing wound healing comprising administering to a patient in need of such enhancement a therapeutically effective amount of the foregoing composition or example thereof.

Another aspect relates to a method for stimulating skeletal muscle regeneration comprising administering to a patient in need of said stimulation a therapeutically effective amount of the foregoing composition or example thereof.

Another aspect relates to a composition including molecules of modified mRNA (modRNA) encoding GATA Binding Protein 4 (G), modRNA encoding Myocyte Enhancer Factor 2C (M), modRNA encoding T-box 5 (T), modRNA encoding Heart- and neural crest derivatives-expressed protein 2 (H), modRNA encoding acid ceramidase (A), modRNA encoding dominant negative transforming growth factor beta (dnT), and modRNA encoding dominant negative Wingless-related integration site 8a (dnW), wherein said molecules of modRNAs are present in said composition in a ratio of G:M:T:H:A:dnT:dnW.

In an example, said ratio is 1:1:1:1:1:1:1. In another example, said ratio is 2:2:2:2:0.7:0.7:0.7. In another example, said ratio is 2:1:1:1:0.7:0.7:0.7. In another example, said ratio is 1:2:1:1:0.7:0.7:0.7. In another example, said ratio is 1:1:2:1:0.7:0.7:0.7. In another example, said ratio is 1:1:1:2:0.7:0.7:0.7. In another example, said ratio is 1:2:1:2:0.5:0.5:0.5. In one example, when the composition comprises a ratio of G:M:T:H:A:dnT:dnW, M is present in an amount that is higher than other modRNA present in said composition. In one example, when the composition comprises a ratio of G:M:T:H:A:dnT:dnW, H is present in an amount that is higher than other modRNA present in said composition.

Another aspect relates to a pharmaceutical composition including a foregoing composition or example thereof and a pharmaceutically acceptable carrier.

Another aspect relates to a method for increasing a ratio of a number of cardiomyocytes to a number of non-cardiomyocytes within a population of cells including contacting said population of cells with the foregoing composition or example thereof. In an example, said non-cardiomyocytes include cardiac fibroblasts.

Another aspect relates to a method for treating cardiac injury including administering to a patient in need of such treatment a therapeutically effective amount of the foregoing composition or example thereof. In an example, the cardiac injury includes myocardial infarction. In another example, the cardiac injury includes reperfusion injury.

Another aspect relates to a method for stimulating vascular regeneration following ischemic damage including contacting tissue damaged by ischemic damage with the foregoing composition or example thereof.

Another aspect relates to a method for treating of stroke including administering to a patient in need of such treatment a therapeutically effective amount of the foregoing composition or example thereof.

Another aspect relates to a method for enhancing wound healing comprising administering to a patient in need of such enhancement a therapeutically effective amount of the foregoing composition or example thereof.

Another aspect relates to a method for stimulating skeletal muscle regeneration comprising administering to a patient in need of said stimulation a therapeutically effective amount of the foregoing composition or example thereof.

Ischemic heart disease remains a major cause of morbidity and mortality in the industrialized world, causing significant societal and economic burden. Reprogramming non-cardiomyocytes (non-CMs) into cardiomyocyte (CM)-like cells in vivo is a promising strategy for cardiac regeneration. However, the current viral-based gene transfer delivery methods have low and erratic transduction efficiency obfuscating translation of these technologies to the clinic. Here, a modified mRNA (modRNA) gene delivery platform is used to deliver 4 cardiac-reprogramming genes (Gata4 (G), Mef2c (M), Tbx5 (T) and Hand2 (H)) together with 3 reprogramming helper genes (Dominant Negative (DN)-TGFβ, DN-Wnt8a and Acid ceramidase (AC)) to induce cardiac reprogramming. It is shown that modRNA cocktail of cardiac reprogramming and helper genes, termed 7G, doubled cardiac reprogramming efficiency (57%) in comparison to conventional Gata4, Mef2C and Tbx5 (GMT) alone (28%). Importantly, repeated 7G modRNA transfection results in beating CMs and complete cardiac reprograming in vitro. Nevertheless, using a lineage-tracing myocardial infraction (MI) mouse model, it is determined that one-time delivery of the 7G modRNA cocktail at the time of MI partially reprogrammed ˜25% of the non-CMs in the scar area. Intriguingly, 7G-modRNA cocktail delivery in mice with MI significantly improved cardiac function, scar size, and long-term survival, and capillary density. Mechanistically, it is shown that 7G modRNA leads to significant upregulation of 15 key angiogenic factors in partial reprogramed cells 28 days post-MI. Also, it is shown that 7G modRNA cocktail leads to neovascularization in ApoE^{-/-} mouse hindlimb ischemia model, indicating that 7G-modRNA cocktail administration promotes vascular regeneration post ischemic injury on the cardiac and skeletal muscle. This approach not only has high efficiency but also high margin of safety for translation to clinic.

In this disclosure, the efficacy of combinatory modRNA cocktail in the direct cardiac reprogramming of non-CMs under ischemic conditions is evaluated. Also evaluated is the beneficial effect of partial cardiac reprogramming in the heart and skeletal muscle, in vivo.

### BRIEF DESCRIPTION OF THE DRAWINGS

These and other features, aspects, and advantages of the present disclosure will become better understood when the following detailed description is read with reference to the accompanying drawings, wherein:

FIG. 1 shows some examples compositions in accordance with aspects of the present disclosure.

FIG. 2 shows effects of different compositions on numbers of myocyte-like cells in accordance with aspects of the present disclosure.

FIG. 3 shows effects of different compositions on cardiac ejection fraction in accordance with aspects of the present disclosure.

FIG. 4 shows effects of different compositions on cardiac output accordance with aspects of the present disclosure.

FIG. 5 shows effects of different compositions on stroke volume in accordance with aspects of the present disclosure.

FIG. 6 shows effects of different compositions on percent change in fractional shortening in accordance with aspects of the present disclosure.

FIG. 7 shows effects of different compositions on percent myocardial infarct size in accordance with aspects of the present disclosure.

FIG. 8 shows effects of different compositions on cardiac capillary density in accordance with aspects of the present disclosure.

FIG. 9 shows effects of different compositions on cardiac VEGF-A expression in accordance with aspects of the present disclosure.

FIGS. 10A-10P illustrate a reprogramming strategy and screen for genes to induce cardiomyocyte reprogramming. FIG. 10A shows strategic illustration of the approach used to test candidate cardiomyocyte-inducing factors. Cardiac cells isolated from the transgenic mice were sorted for mCherry-negative cells to eliminate cardiomyocytes. FIG. 10B shows experimental timeline for western blot (WB). FIG. 10C shows WB analysis for mCherry-negative cells transfected with GFP, GATA4, MEF2C, TBX5 modRNA to access the transfection strategy to reprogram non-cardiomyocytes into cardiomyocytes. FIG. 10D is a schematic representation of timeline used to transfect mCherry-negative cells with different reprogramming genes. FIGS. 10E and 10F are graphs showing expression of major cardiac genes (cTNT and α-MHC) determined by quantitative polymerase chain reaction (qPCR) of RNA extracted from non-CMs reprogrammed for 14 days with Gata4, Mef2c and Tbx5 (GMT) or GMT plus Hand2 (GMTH) or GMTH plus AC (GMTHA) and small molecule inhibitors (SMI) (a n=10, b n=7, c n=5 and d n=6). FIG. 10G shows representative immunostaining images of cells transfected with reprogramming genes plus SMI for cardiac markers α-MHC/α-actinin. FIG. 10H shows percentage quantification of 10G. FIGS. 10I and 10J show the mRNA expression of cTNT and α-MHC in mCherry-negative cells evaluated 14 days after GMTHA+SMI and GMTHA plus SMI replacement gene transfections, determined by qPCR (a n=10, d n=6, e n=8, f n=4, g n=4 and h n=4). FIG. 10K shows immunofluorescent staining for α-actinin in mCherry-negative cells 14 days after first transfection with GMTHA+SMI and GMTHA plus SMI replacement genes. FIG. 10L shows percentage quantification of 10K. FIG. 10M shows relative expression of fibroblast markers in non-CMs after 7G transfection for 14 days (Luc n=4, 7G n=4). FIG. 10N shows 7G transfection strategy for mCherry-negative cells post 14 days. FIGS. 10O and 10P show representative immunostaining images for mCherry-negative cells showing striations of α-actinin post 21- and 28-days transfection (respectively) with 7G. One-way ANOVA, Tukey's Multiple Comparison Test for FIGS. 10E and 10F, FIGS. 10I and 10J, FIGS. 10H and 10L. Unpaired two-tailed t-test form. ****, P<0.0001, **, P<0.01, N.S, Not-Significant. Scale bar=10 μm.

FIGS. 11A-11F show that varied stoichiometry among 7 genes influences the reprogramming efficiency of cardiac fibroblasts in vitro. FIG. 11A is a schematic representation of isolating mCherry-negative cells from α-MHC transgenic mice, using FACS sorting, and transfecting them at 3-day intervals with different ratios of 7 genes for cardiac reprogramming experiments. FIG. 11B is a table showing transfection groups with different ratios of 7 genes. FIGS. 11C and 11D show the mRNA relative expression of cTNT and αMHC, respectively, evaluated in mCherry-negative cells 14 days post transfection with different gene ratios, determined by qPCR (a n=10, b n-8, c n-2, d n-2, e n-2, f n-2, g n-2, h n-2, I n-2 and j n=4). FIG. 11E shows immunostaining images of mCherry-negative cells that exhibited αMHC-mCherry and α-actinin after 2 weeks of transfection with reprogramming genes. FIG. 11F shows percentage quantification of e. One-way ANOVA, Tukey's Multiple Comparison Test for c&d and f ***, P<0.001, **, P<0.01, N.S, Not-Significant. Scale bar=10 μm.

FIGS. 12A-12O illustrate that 7G improves cardiac function and outcome post MI. FIG. 12A is an experimental timeline to evaluate cardiovascular function after delivery of Luc and 7G modRNAs in an acute MI mouse model. FIG. 12B shows magnetic resonance imaging (MRI) assessments of left ventricular systolic and diastolic function, performed 28 days post MI. Representative images show the left ventricular chamber (outlined in red) during diastole and systole. FIGS. 12C, 12D, 12E, and 12F show percentage evaluation of ejection fraction (EF), cardiac output (CO) and myocardial infarction (MI) size measured by MRI experiments in b (Luc n=3, 7G n=3). FIG. 12G shows percentage of fractioning shortening differences, obtained by echocardiography, between day 2 (baseline) and day 28 post MI (Luc n=17, 7G n=8). FIGS. 12H and 12I show measurement of differences in left ventricular internal diameter diastole (LVIDd) and systole (LVIDs), evaluated by echocardiography, between day 2 (baseline) and day 28 post-MI (Luc n=17, 7G n=8). FIG. 12J shows an experimental plan for isolating hearts for scar tissue assessment and qPCR analysis for fibrosis markers. FIG. 12K shows representative histological sections with Masson's trichrome staining to evaluate scar size 28 days post MI. FIG. 12L shows percentage quantification of left ventricle area occupied by scar vs. viable tissue 28 days post MI, based on j (Luc n=3, 7G n=3). FIG. 12M is a qPCR panel showing relative expression of fibrosis markers comparing Luc treatment with 7G modRNA, 28 days post MI. FIGS. 12N and 12O show long-term survival curve after myocardial infarction and 7G and 7G modRNA compared to Luc treatment (Luc n=12, 7G n=12). Unpaired two-tailed t-test for c-g and l&m. Two-way ANOVA, Bonferroni post-hoc test for FIGS. 12H and 12I. Mantel-Cox log-rank test for o. ****, P<0.0001, ***, P<0.001, **, P<0.01, *, P<0.05, N.S, Not Significant. Scale bar=1 mm (b&k).

FIGS. 13A-13J show that 7G improves cardiac function by promoting angiogenesis. FIG. 13A shows an experimental plan for tamoxifen injection, LAD ligation and modRNA delivery followed by isolation of hearts from transgenic mice. FIG. 13B shows representative immunostaining images of in vivo CM-like cells (presented in yellow) collected from Luc and 7G modRNA-injected mice, showing positive stain for cardiomyocyte marker cTNT (green) and Tdtomato-cre. FIG. 13C shows quantification of CM-like cells depicted by immunostaining in B (Luc n=3, 7G n=3). FIG. 13D is an experimental model for analyzing endothelial cells. FIG. 13E shows representative immunofluorescence showing the presence of luminal structure (green) in the scar area 28 days after delivery of Luc and 7G modRNA. FIG. 13F shows percentage quantification of capillary density based on e (Luc n=5, 7G n=4). FIG. 13G shows a strategic plan for modRNA injection and collection of hearts for mRNA and protein analysis post MI. FIG. 13H shows qPCR quantification of the expression of major angiogenic genes determined evaluate from RNA extracted from cardiac tissue 28 days post modRNA treatment (n=2). FIG. 13I shows exemplary Western blot images showing the presence of VEGFA protein in cardiac tissue isolated 4 weeks post MI and subsequent modRNA injection of Luc and 7G. FIG. 13J shows quantification of western blot (n=2). Unpaired two-tailed t-test for c, f, j, h. ****, P<0.0001, ***, P<0.001, **, P<0.01, N.S, Not Significant. Scale bar=50 μm (b), 25 μm (e).

FIGS. 14A-14E illustrate that 7G enhances blood perfusion and angiogenesis in ApoE−/− mouse hindlimb ischemia model. FIG. 14A is an experimental plan for femoral artery ligation and modRNA delivery followed blood perfusion analysis from the ApoE−/− mice. FIG. 14B is a representative Laser Doppler perfusion imaging (LDPI) showing dynamic changes in blood perfusion in the foot region of mouse which received Luc and 7G modRNA post femoral artery ligation at days 0, 1, 7, 14 and 21. FIG. 14C shows quantification of foot perfusion measured by LDPI based on FIG. 14C where the ratio of LDPI is calculated by comparing the blood perfusion in ischemic limb to its contralateral hind limb (Luc n=5, 7G n=5). FIG. 14D shows an experimental timeline for performing qPCR and immunostaining on the mouse gastrocnemius muscle. FIG. 14E is a qPCR panel showing the upregulation of angiogenic genes in the ischemic muscle isolated from the mouse injected with Luc and 7G post critical limb ischemia. Two-way ANOVA, Bonferroni post-hoc test for FIG. 14C. Unpaired two-tailed t-test for FIG. 14E. ****, P<0.0001, **, P<0.01, *, P<0.05, N.S, Not Significant.

FIG. 15 shows a proposed model for 7G modRNA induction of vascular regeneration in heart and limb models post ischemic injury.

FIGS. 16A-16K show that a cocktail of 7 Genes+Myocardin delivered by modRNA induces cardiac gene expression in adult human cardiac fibroblasts. FIG. 16A is an experimental timeline for performing protein analysis in normal human ventricular cardiac fibroblasts (NHCF-V) transfected with different modRNAs. FIG. 16B shows a western blot analysis of GFP, GATA4, MEF2C, TBX5 modRNA-transfected NHCF-V at different time points (Day 1-Day 6) to determine the protein expression length. FIG. 16C shows an experimental plan for NHCF-V transfection and cell collection to determine the efficiency of different transfection reagents. FIG. 16D shows representative images of immunostaining performed on NHCF-V, showing nGFP expression, transfected with various transfection reagents. FIG. 16E shows percentage quantification analysis of nGFP-positive cells based on immunostaining FIG. 16D. FIG. 16F shows an experimental plan for transfecting and collecting SV40-T-pretreated NHCF-V. FIGS. 16G, 16H, and 16I show relative expression of cTNT, α-MHC and COLA1, respectively, determined by qPCR analysis on NHCF-V collected after 14 days of repeated transfections with different reprogramming gene cocktails (a n=3, b n=3, c n=2, d n=2, e n=2, f n=2). FIG. 16J shows representative immunofluorescence images showing the appearance of a cardiomyocyte marker (α-actinin) in NHCF-V reprogrammed with different cocktails of genes. (Green: α-actinin, blue: DAPI; scale bar, 50 μm). FIG. 16K shows quantification of the percentage of cells that exhibited α-actinin after 2 weeks of transfection with reprogramming genes. One-way ANOVA, Tukey's Multiple Comparison Test for FIGS. 16E, 16G, 16H, 16I, 16K. ****, P<0.0001, ***, P<0.001, **, P<0.01, *, P<0.05, N.S, Not-Significant. Scale bar=10 μm.

FIGS. 17A-17F show that adding other candidates to 7G does not make cardiac reprogramming more efficient. FIG. 17A shows a representative diagram of experimental plan demonstrating the use of FACS sorting to isolate mCherry-negative cells from transgenic mice, plating the cells and transfections every 3 days with different additions to the 7-gene cocktail. FIG. 17B shows a list of genes screened in addition to the 7 genes and their ratios used in transfecting mCherry-negative cells to identify the best cocktail for reprogramming fibroblasts into cardiomyocytes. FIGS. 17C and 17D show qPCR results comparing the relative gene expression of cTNT and αMHC, respectively, in mCherry-negative cells 14 days after transfection with 7 genes or 7 genes plus the additional candidates (a n=10, b n=8, c n=4, d n=4, e n-4, f n-4, g n-2, h n-2, l n-2 and j n=2, k n=4, 1 n=4, m n=4, n=4 and o n=4). FIG. 17E shows representative images of α-actinin- and αMHC-mCherry-positive cells in mCherry-negative sorted cells, acquired 14 days after repeated transfections of different groups of genes. FIG. 17F shows percentage quantification based on FIG. 17E. One-way ANOVA, Tukey's Multiple Comparison Test for FIGS. 17D, 17E, and 17F. ****, P<0.0001, ***, P<0.001, **, P<0.01, *, P<0.05, N.S, Not-Significant. Scale bar=20 μm.

FIGS. 18A-18D show the effect of different stoichiometry of 7G on cardiac function and outcome post MI. FIG. 18A shows experimental design to assess cardiovascular function post MI. Luc, 7G GMT(Hx2) and 7G G(Mx2)TH modRNA were injected into the myocardium at the time of LAD ligation. FIG. 18B shows percentage quantification of delta in fractional shortening (Day 28-Day 2) based on echocardiography in mice injected with 7G GMT(Hx2) and 7G G(Mx2)TH modRNA. FIG. 18C shows representative Masson's trichrome staining to evaluate scar size 28 days post MI. FIG. 18D shows quantification of scar size based on the images shown in c (Luc n=3, 7G GMT(Hx2) n=3, 7G G(Mx2)TH n=3). One-way ANOVA, Tukey's Multiple Comparison Test for FIGS. 18C and 18D. ****, P<0.0001, **, P<0.01, N.S, Not-Significant. Scale bar=1 mm for b.

FIGS. 19A-19B show lineage-tracing mouse model to identify CM-like cell formation in vivo. FIG. 19A shows an overview of crossing in mouse model used in lineage-tracing experiments. FIG. 19B shows representative immunostaining images of Tnnt2^{MerCreMer/+}/R26^{mTmG/+} collected from Tnnt2^{MerCreMer/+}/R26^{mTmG/+} mouse hearts, comparing the appearance of membrane green in Tnnt2-positive cells in sham vs. tamoxifen-treated mice. Scale bar=50 μm for b.

FIGS. 20A-20H show that 7G GMT(Hx2) enhances the differentiation of fibroblasts into CM-like cells and promotes angiogenesis in mouse heart post MI. FIG. 20A shows an experimental design to assess CM reprogramming in the lineage tracing mouse model. FIG. 20B shows immunostaining images of in vivo reprogrammed cells (presented in yellow) collected from Luc and 7G GMT(Hx2) modRNA-injected mice post MI. FIG. 20C shows CM like cells quantification based on FIG. 20B. FIG. 20D shows an experimental model for analyzing endothelial cells. FIG. 20E shows representative immunostaining images of CD31 positive cells in the scar region of hearts that underwent MI and subsequent Luc and 7G GMT(Hx2) modRNA treatment. FIG. 20F shows percentage quantification of CD31 positive cells. FIG. 20G shows a strategic plan for modRNA injection and collection of hearts for qPCR analysis. FIG. 20H is a qPCR panel showing relative expression of angiogenic markers comparing Luc treatment with 7G GMT(Hx2) modRNA in an acute MI mouse model at days 28 (Luc n=2 and 7G GMT (Hx2) n=2). Unpaired two-tailed t-test for FIGS. 20C, 20F, and 20H. ****, P<0.0001, ***, P<0.001, **, P<0.01, N.S, Not Significant. Scale bar=50 μm (b&e).

FIGS. 21A-21G show results of a weekly assessment of angiogenic and fibroblast markers following intracardiac delivery of modRNA. FIG. 21A shows an experimental design for LAD ligation, subsequent modRNA injection and heart isolation for qPCR and western blot. FIGS. 21B, 21C, and 21D show qPCR panel showing relative expression of angiogenic markers comparing Luc treatment with 7G and 7G GMT(Hx2) modRNA in an acute MI mouse model at days 7, 14 and 21 (Luc n=2, 7G n=2, 7G GMT (Hx2) n=2). FIGS. 21E, 21F, and 21G show known fibrosis markers determined by qPCR at days 7, 14 and 21(Luc n=2, 7G n=2, 7G GMT (Hx2) n=2). FIG. 21H is a western blot analysis of VEGFA protein levels in mouse heart samples collected at Day 21. FIG. 21I show quantification of FIG. 21H. One-way ANOVA, Tukey's Multiple Comparison Test for FIGS. 21B-21I. ****, P<0.0001, ***, P<0.001, **, P<0.01, *, P<0.05, N.S, Not-Significant.

FIGS. 22A-22E show that increased Vegfa level is not associated with leaky vessels or angioma in the heart. FIG. 22A shows an experimental timeline to investigate the association of increased Vegfa levels with tissue perfusion and leaky vessels. FIG. 22B is a graph showing a weekly assessment of Height weight/Body weight ratio quantified for 3 treatment groups. FIG. 22C shows whole heart images to evaluate heart size and myocardial edema in hearts treated with Luc, 7G or 7G GMT(Hx2) modRNA. FIG. 22D is representative images of H&E staining revealing no angioma associated with Luc, 7G or 7G GMT(Hx2) treatment 28 days post MI. FIG. 22E shows representative immunostaining images of mouse hearts, either sham or treated with Luc, 7G and 7G GMT(Hx2) at the time of LAD ligation, to evaluate the association between treatments and systemic vasculature (isolectin B4) or vascular permeability (FITC-dextran beads (70 kDa)). One-way ANOVA, Tukey's Multiple Comparison Test for b. N.S, Not-Significant. Scale bar=1 mm (FIGS. 22C, 22D), 10 μm (FIG. 22E).

FIGS. 23A-23D show Mouse and human CM-like cells significantly upregulate the expression of VegfA, a key angiogenic gene. FIG. 23A shows a schematic representation of procedure to isolate mCherry-negative cells from α-MHC transgenic mice, using FACS sorting, and transfect them at 3-day intervals with or without different cardiac reprogramming cocktails (7G, 7G GMT(Hx2)). FIG. 23B shows relative mRNA expression of VegfA via qPCR evaluation of mCherry-negative cells 14 days post transfections with or without 7G or 7G GMT(Hx2) (Luc n=3, 7G n=3, 7G GMT (Hx2) n=3). FIG. 23C shows an experimental timeline for treating and collecting NHCF-V for qPCR. FIG. 23D is a graph representing VEGFA expression, as determined by qPCR of RNA extracted from human cardiac fibroblast cells reprogramed for 14 days with or without 7G+Myocd (Luc n=3, 7G GMT (Hx2) n=3). One-way ANOVA, Tukey's Multiple Comparison Test for b. Unpaired two-tailed t-test for d. ***, P<0.001, *, P<0.05, N.S, Not-Significant.

### DETAILED DESCRIPTION

A first aspect relates to a composition including molecules of modified mRNA (modRNA) encoding GATA Binding Protein 4 (G), modRNA encoding Myocyte Enhancer Factor 2C (M), modRNA encoding T-box 5 (T), modRNA encoding Heart- and neural crest derivatives-expressed protein 2 (H), modRNA encoding dominant negative transforming growth factor beta (dnT), and modRNA encoding dominant negative Wingless-related integration site 8a (dnW), wherein said molecules of modRNAs are present in said composition in a ratio of G:M:T:H:dnT:dnW.

Another aspect relates to a composition including molecules of modified mRNA (modRNA) encoding GATA Binding Protein 4 (G), modRNA encoding Myocyte Enhancer Factor 2C (M), modRNA encoding T-box 5 (T), modRNA encoding Heart- and neural crest derivatives-expressed protein 2 (H), modRNA encoding acid ceramidase (A), modRNA encoding dominant negative transforming growth factor beta (dnT), and modRNA encoding dominant negative Wingless-related integration site 8a (dnW), wherein said molecules of modRNAs are present in said composition in a ratio of G:M:T:H:A:dnT:dnW.

It is to be appreciated that certain aspects, modes, embodiments, variations and features of the present invention are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this invention belongs.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the terms “subject,” “individual” or “patient,” used interchangeably, means any animal, including mammals, such as mice, rats, other rodents, rabbits, dogs, cats, swine, cattle, sheep, horses, or primates, such as humans.

As used herein, the term “purified” means that when isolated, the isolate contains at least 90%, at least 95%, at least 98%, or at least 99% of a compound described herein by weight of the isolate.

As used herein, the phrase “substantially isolated” means a compound that is at least partially or substantially separated from the environment in which it is formed or detected.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable sub-combination.

The term “cell or group of cells” is intended to encompass single cells as well as multiple cells either in suspension or in monolayers. Whole tissues also constitute a group of cells.

Duration of expression can be tailored to the specific situation by choice of gene delivery method. The term “short term expression,” for example, refers to expression of the desired protein for a duration of several days rather than weeks. So, for example, the use of modRNA as a gene delivery method achieves transient expression of the selected sphingolipid-metabolizing protein for up to about 11 or 12 days. Quick, transient expression of short duration may be sufficient, for example, to extend survival and the quality of oocytes and embryos prior to IVF.

The term “modRNA” refers to a synthetic modified RNA that can be used for expression of a gene of interest. Chemical modifications made in the modRNA, for example substitution of pseudouridine for uridine, stabilize the molecule and enhance transcription. Additionally, unlike delivery of protein agents directly to a cell, which can activate the immune system, the delivery of modRNA can be achieved without immune impact. The use of modRNA for in vivo and in vitro expression is described in more detail in for example, WO 2012/138453, which is hereby incorporated by reference in its entirety.

The discovery by Kariko et al., “Incorporation of Pseudouridine Into mRNA Yields Superior Nonimunogenic Vector With Increased Translational Capacity and Biological Stability,” Mol. Ther. 16(11):1833-1840 (2008), which is hereby incorporated by reference in its entirety) that the substitution of uridine and cytidine with pseudouridine and 5-methylcytidine, respectively, drastically reduced the immune response elicited from exogenous RNA set the stage for a new kind of gene delivery, in which transient expression of therapeutic proteins is achieved.

Modified mRNA (modRNA) is a relatively new gene delivery system, which can be used in vitro or in vivo to achieve transient expression of therapeutic proteins in a heterogeneous population of cells. Incorporation of specific modified nucleosides enables modRNA to be translated efficiently without triggering antiviral and innate immune responses. In the present disclosure, modRNA is shown to be effective at delivering short-term robust gene expression of a “survival gene” and its use in the field of gene therapy is expanding. A stepwise protocol for the synthesis of modRNA for delivery of therapeutic proteins is disclosed in, for example, Kondrat et al., “Synthesis of Modified mRNA for Myocardial Delivery,” Cardiac Gene Therapy 1521:127-138 (2017), which is hereby incorporated by reference in its entirety.

modRNA, a relatively nascent technology, has considerable potential as a therapy for disease. Delivery of a synthetic modified RNA encoding human vascular endothelial growth factor-A, for example, results in expansion and directed differentiation of endogenous heart progenitors in a mouse myocardial infarction model (Zangi et al., “Modified mRNA Directs the Fate of Heart Progenitor Cells and Induces Vascular Regeneration After Myocardial Infarction,” Nature Biotechnology 31:898-907 (2013), which is hereby incorporated by reference in its entirety). In another example, diabetic neuropathy may be lessened by the ability to deliver genes encoding nerve growth factor. Additionally, with the advent of genome editing technology, CRISPR/Cas9 or transcription activator-like effector nuclease (TALEN), transfection will be safer if delivered in a transient and cell-specific manner.

In an example in accordance with the present disclosure, a gene delivery molecule that encodes a protein that may influence development of cardiomyocytes or adoption of a cardiomyocyte-like phenotype includes a modRNA. While various gene delivery methods exist for achieving expression of an exogenous protein, for example, using plasmids, viruses or mRNA, in certain situations modRNA offers several advantages as a gene delivery tool.

An advantage of gene delivery over protein may be the ability to achieve endogenous expression of protein for a specific period of time and therefore extended exposure to a protein translation product of interest. Another advantage of modRNA delivery may be the lack of a requirement for nuclear localization or transcription prior to translation of the gene of interest. Reducing or avoiding transcription of an mRNA before translation of the protein of interest may result in higher efficiency in expression of the protein of interest.

Kariko et al. showed in 2008 that uridine replacement in mRNA with pseudouridine (hence the name modified mRNA (modRNA)) resulted in changes to the mRNA secondary structure that avoid the innate immune system and reduce the recognition of modRNA by RNase. Kariko et al., “Incorporation of Pseudouridine Into mRNA Yields Superior Nonimunogenic Vector With Increased Translational Capacity and Biological Stability,” Mol. Ther. 16(11):1833-1840 (2008), which is hereby incorporated by reference in its entirety. In addition, these changes of nucleotides are naturally occurring in our body and lead to enhance translation of the modRNA compared to unmodified mRNA.

Disclosed herein is a modRNA “cocktail” including modRNAs that encode one or more proteins that influence development of cardiomyocytes or adoption of a cardiomyocyte-like phenotype or promote cardiomyocyte survival, and administration of such cocktail to promote development of non-cardiomyocyte cells into cardiomyocytes or to adopt a cardiomyocyte-like phenotype, or promote cardiac health or function, diminish cardiac damage or impairment that may otherwise follow from cardiac injury or insult.

modRNA is a synthetic mRNA and may include an optimized 5′UTR and 3′UTR sequences, anti-reverse cup analog (ARCA), one or more naturally modified nucleotides, or any combination of the foregoing. Optimized UTRs sequences may enhance the translation efficiency. ARCA may increase the stability of the RNA and enhances the translation efficiency and the naturally modified nucleotides increase the stability of the RNA reduce the innate immune response of cells (in vitro and in vivo) and enhance the translation efficiency of the mRNA. In other examples, mRNA may be treated with a reagent that promotes adoption of a Cap 1 structure, which promotes evasion of an mRNA-directed immune response. modRNA (such as mRNA containing pseudouridine in place of uridine or other ribonucleotide substitutions) may be treated with such a reagent such that it may adopt a 5′UTR Cap 1 structure. For example, treating a mRNA with a commercially available reagent such as CLEANCAP™ (TriLink Biotechnologies) may promote formation of a 5′UTR Cap 1 structure and thereby may mediate a higher and longer expression of proteins with a reduced or minimized immune response.

This disclosure relates to a composition for increasing a number of cardiomyocytes within a population of cells by modifying activity of cellular proteins. By increasing or decreasing activity of proteins or signaling pathways that may be involved in development of a phenotype characteristic of cardiomyocytes, cells within the population that do not have a cardiomyocyte-like phenotype may be induced to develop such a phenotype and thereby become cardiomyocytes. Increased number of cardiomyocytes may then improve cardiac function, including repair or regeneration following insult or injury or prevention of disadvantageous sequelae of insult or injury and thereby lead to improved health or function compared to absence of treatment. In other examples, treatment with a composition may prevent a decrease in cardiomyocytes, or reduce cells that would otherwise promote scarring or poor cardiac function or physiology such as, in an example, by promoting transformation of such cells into cardiomyocytes.

A number of different cellular factors, proteins, or signaling factors may function to promote transformation of a cell from a non-cardiomyocyte phenotype to a cardiomyocyte phenotype, or otherwise be involved in development of a cell into a cardiomyocyte, or otherwise promote the health or survival of cardiomyocytes or cells with a cardiomyocyte-like phenotype. Examples may include GATA Binding Protein 4 (GATA4), Myocyte Enhancer Factor 2C (Mef2c), T-box 5 (Tbx5), Heart- and neural crest derivatives-expressed protein 2 (Hand2), acid ceramidase (ASAH1 gene that encodes acid ceramidase), transforming growth factor beta (TGFB), and Wingless-related integration site 8a (Wnt8a).

Cardiac fibroblasts, which may represent 50% of cells in the mammalian heart, may be directly reprogrammed to cardiomyocyte-like cells in vitro by increasing their expression of the developmental cardiac regulators GATA4, Mef2c and Tbx5. Ieda et al., “Direct Reprogramming of Fibroblasts into Functional Cardiomyocytes by Defined Factors,” Cell 142:375-386 (2010), which is hereby incorporated by reference in its entirety. Furthermore, inhibition of TGFB with small molecule inhibitors, such as SB431542, enhances differentiation of cardiomyocytes, as does inhibition of Wnt signaling with the small molecule inhibitor XAV939. Increased expression of ASAH1 may also promote health, growth, or survival of cardiomyocytes.

However, the potential for complex interrelationships between modification of these factors' activity has hampered development of a combinatorial approach for enhanced cardiomyocyte formation or development, such as following cardiac injury or insult, or for increasing cardiomyocyte levels within a population of cells.

Hand2 has also been demonstrated to promote cardiomyocyte formation, such as in zebrafish. Schindler et al., “Hand2 Elevates Cardiomyocyte Production During Zebrafish Heart Development and Regeneration,” Development 141:3112-3122 (2014), which is hereby incorporated by reference in its entirety. However, whether Hand2 may cooperate with one or more of the aforementioned cellular proteins in promoting development of cardiomyocytes, or transformation of non-cardiomyocyte cells into cardiomyocytes or calls having a cardiomyocyte-like phenotype, is not known. And as with the foregoing cellular influences on cardiomyocyte development, relative ratios of stimulating such factors as promote cardiomyocyte development or survival and/or inhibiting such factors as promote cardiomyocyte development or survival for combinatorial treatment to promote cardiomyocyte development and cardiac health following injury or insult is not known.

As disclosed herein, a composition including modRNA encoding GATA4, Mef2c, Tbx5, and Hand2, together with modRNA encoding peptides that inhibit activity of TGFB and Wnt8a, so-called “dominant negative” TGFB and “dominant negative” Wnt8a (dnTGFB and dnWnt8a, respectively), increases a number of cardiomyocytes within a population of cells including non-cardiomyocytes. In an example, non-cardiomyocytes include cardiac fibroblasts. In another example, such composition includes modRNA encoding ASAH1. In an example, a composition may be applied to cells including non-cardiomyocytes resulting in an increase in a number of cardiomyocytes present within the cell population.

In one embodiment, a ratio of GATA4 modRNA molecules:Mef2c modRNA molecules:Tbx5 modRNA molecules:Hand2 modRNA molecules:dnTGFB modRNA molecules:dnWnt8a modRNA molecules is 1:1:1:1:1:1, or 2:1:1:1:0.7:0.7, or 1:2:1:1:0.7:0.7, or 1:1:2:1:0.7:0.7, or 1:1:1:2:0.7:0.7, or 1:2:1:2:0.5:0.5, or 2:2:2:2:0.7:0.7. In another embodiment, a ratio of GATA4 modRNA molecules:Mef2c modRNA molecules:Tbx5 modRNA molecules:Hand2 modRNA molecules:ASAH1 mod RNA molecules:dnTGFB modRNA molecules:dnWnt8a modRNA molecules is 1:1:1:1:1:1:1, or 2:1:1:1:0.7:0.7:0.7, or 1:2:1:1:0.7:0.7:0.7, or 1:1:2:1:0.7:0.7:0.7, or 1:1:1:2:0.7:0.7:0.7, or 1:2:1:2:0.5:0.5:0.5, or 2:2:2:2:0.7:0.7:0.7.

In one embodiment, when the composition comprises a ratio of G:M:T:H:dnT:dnW, M is present in an amount that is higher than other modRNA present in said composition. In another embodiment, when the composition comprises a ratio of G:M:T:H:dnT:dnW, H is present in an amount that is higher than other modRNA present in said composition. In one embodiment, when the composition comprises a ratio of G:M:T:H:A:dnT:dnW, M is present in an amount that is higher than other modRNA present in said composition. In another embodiment, when the composition comprises a ratio of G:M:T:H:A:dnT:dnW, H is present in an amount that is higher than other modRNA present in said composition.

Sense DNA sequences corresponding to modRNA molecules disclosed herein are presented in Table 1.

TABLE 1
Sense DNA sequences

| | SEQ | |
|---|---|---|
| | ID | |
| Molecule | NO | DNA sense strand sequence |
| Mouse | 1 | ATGTACCAAAGCCTGGCCATGGCCGCCAACCACGGCCCCCCGCCCGGCGCCTACGAAGC |
| GATA4 | | AGGTGGCCCTGGCGCCTTCATGCACAGCGCGGGCGCCGCGTCCTCGCCCGTCTACGTGC |
| | | CCACTCCGCGGGTGCCGTCCTCTGTGCTGGGCCTGTCCTACCTGCAGGGCGGTGGCAGT |
| | | GCCGCTGCAGCTGGAACCACCTCGGGTGGCAGCTCCGGGGCCGGCCCGTCGGGTGCAGG |
| | | GCCTGGGACCCAGCAGGGTAGCCCTGGCTGGAGCCAAGCTGGAGCCGAGGGAGCCGCCT |
| | | ACACCCCGCCGCCCGTGTCCCCGCGCTTCTCTTTCCCGGGGACTACTGGGTCCCTGGCG |
| | | GCCGCTGCCGCCGCTGCCGCAGCCCGGGAAGCTGCAGCCTACGGCAGTGGCGGCGGGGC |
| | | GGCGGGCGCTGGTCTGGCTGGCCGAGAGCAGTACGGGCGTCCGGGCTTCGCCGGCTCCT |
| | | ACTCCAGCCCCTACCCAGCCTACATGGCCGACGTGGGAGCATCCTGGGCCGCAGCCGCT |
| | | GCCGCCTCTGCCGGCCCCTTCGACAGCCCAGTCCTGCACAGCCTGCCTGGACGGGCCAA |
| | | CCCTGGAAGACACCCCAATCTCGTAGATATGTTTGATGACTTCTCAGAAGGCAGAGAGT |
| | | GTGTCAATTGTGGGGCCATGTCCACCCCACTCTGGAGGCGAGATGGGACGGGACACTAC |
| | | CTGTGCAATGCCTGTGGCCTCTATCACAAGATGAACGGCATCAACCGGCCCCTCATTAA |
| | | GCCTCAGCGCCGCCTGTCCGCTTCCCGCCGGGTAGGCCTCTCCTGTGCCAACTGCCAGA |
| | | CTACCACCACCACGCTGTGGCGTCGTAATGCCGAGGGTGAGCCTGTATGTAATGCCTGC |
| | | GGCCTCTACATGAAGCTCCATGGGGTTCCCAGGCCTCTTGCAATGCGGAAGGAGGGGAT |
| | | TCAAACCAGAAAACGGAAGCCCAAGAACCTGAATAAATCTAAGACGCCAGCAGGTCCTG |
| | | CTGGTGAGACCCTCCCTCCCTCCAGTGGTGCCTCCAGCGGTAACTCCAGCAATGCCACT |
| | | AGCAGCAGCAGCAGCAGTGAAGAGATGCGCCCCATCAAGACAGAGCCCGGGCTGTCATC |
| | | TCACTATGGGCACAGCAGCTCCATGTCCCAGACATTCAGTACTGTGTCCGGCCACGGGC |
| | | CCTCCATCCATCCAGTGCTGTCTGCTCTGAAGCTGTCCCCACAAGGCTATGCATCTCCT |
| | | GTCACTCAGACATCGCAGGCCAGCTCCAAGCAGGACTCTTGGAACAGCCTGGTCCTGGC |
| | | TGACAGTCATGGGGACATAATCACCGCGTAA |
| Mouse | 2 | ATGGGGAGAAAAAAGATTCAGATTACGAGGATAATGGATGAGCGTAACAGACAGGTGAC |
| Mef2c | | TTTTACGAAGAGGAAATTTGGATTGATGAAGAAGGCTTATGAGCTGAGCGTGCTGTGCG |
| | | ACTGTGAGATTGCACTGATCATCTTCAACAGCACCAACAAGCTGTTCCAGTACGCCAGC |
| | | ACTGACATGGATAAGGTGTTGCTCAAGTACACCGAGTACAACGAGCCGCACGAGAGCCG |
| | | GACAAACTCAGACATTGTGGAGACATTGAGAAAGAAGGGCCTCAATGGCTGTGACAGCC |
| | | CAGATCCCGATGCAGACGATTCAGTAGGTCACAGCCCTGAGTCTGAGGACAAGTACAGG |
| | | AAAATTAACGAAGATATTGATCTAATGATCAGCAGGCAAAGATTGTGTGCTGTTCCACC |
| | | TCCCAGCTTTGAGATGCCAGTTACCATCCCAGTGTCCAGCCATAACAGTTTGGTGTACA |
| | | GCAATCCTGTCAGCACACTGGGAAACCCCAATCTTCTGCCACTGGCCCACCCGTCTCTG |
| | | CAGAGGAATAGTATGTCTCCTGGTGTAACACATAGACCTCCAAGTGCAGGTAACACAGG |
| | | CGGTCTGATGGGCGGAGATCTGACATCCGGTGCAGGCACCAGCGCAGGGAATGGATACG |
| | | GCAACCCCCGGAACTCACCAGGCCTGCTGGTCTCACCTGGTAACCTGAACAAGAATATA |
| | | CAAGCCAAATCTCCTCCCCCTATGAATCTAGGAATGAATAATCGTAAGCCAGATCTCCG |
| | | CGTTCTTATCCCACCTGGCAGCAAGAACACGATGCCATCAGTGAATCAAAGGATAAATA |
| | | ACTCCCAGTCGGCTCAGTCATTGGCTACCCCGGTGGTTTCCGTAGCAACTCCTACTTTA |
| | | CCAGGACAAGGAATGGGAGGATATCCATCAGCCATTTCAACAACATATGGTACTGAGTA |
| | | CTCTCTGAGTAGCGCAGATCTGTCATCTCTGTCTGGCTTCAACACTGCCAGTGCGCTCC |
| | | ACCTCGGCTCTGTAACTGGCTGGCAGCAGCAGCACCTACATAACATGCCGCCATCTGCC |
| | | CTCAGTCAGTTGGGAGCTTGCACTAGCACTCATTTATCTCAGAGTTCAAATCTCTCCCT |
| | | GCCTTCTACTCAAAGCCTCAGCATCAAGTCAGAACCTGTTTCTCCTCCTAGAGACCGTA |
| | | CCACCACCCCTTCGAGATACCCACAACACACCACGCGCCACGAGGCGGGGAGGTCTCCT |
| | | GTTGACAGCTTGAGCAGCTGTAGCAGTTCCTACGATGGGAGCGACCGAGAGGATCACCG |
| | | GAACGAATTCCACTCCCCCATTGGACTCACCAGACCTTCGCCGGACGAAAGGGAAAGTC |
| | | CTTCAGTCAAGCGCATGCGACTCTCTGAAGGATGGGCAACATGA |
| Mouse | 3 | ATGGCCGATACAGATGAGGGCTTTGGCCTGGCGCGCACGCCTCTGGAGCCTGATTCCAA |
| Tbx5 | | AGACAGGTCTTGCGATTCGAAACCTGAGAGTGCTCTGGGGGCTCCCAGCAAGTCTCCAT |
| | | CATCCCCGCAGGCTGCCTTCACCCAGCAGGGCATGGAAGGAATCAAGGTGTTTCTTCAT |
| | | GAACGTGAACTGTGGCTGAAGTTCCACGAAGTGGGCACAGAGATGATCATCACCAAGGC |
| | | AGGGAGGAGAATGTTTCCTAGTTACAAAGTGAAGGTGACTGGCCTTAATCCCAAAACGA |
| | | AGTATATTCTTCTCATGGATATTGTTCCCGCAGACGACCACAGATATAAATTTGCTGAT |
| | | AACAAATGGTCCGTAACTGGCAAAGCAGAGCCTGCCATGCCGGGGCGCCTTTACGTGCA |
| | | CCCGGACTCCCCAGCAACCGGAGCCCACTGGATGCGACAACTTGTCTCCTTCCAGAAGC |
| | | TCAAACTCACCAACAACCACCTGGACCCGTTTGGACACATTATCCTGAACTCCATGCAC |
| | | AAATACCAGCCCCGATTACACATCGTGAAAGCAGACGAAAATAATGGGTTCGGTTCAAA |
| | | GAACACTGCGTTTTGCACCCACGTCTTCCCGGAGACAGCTTTTATCGCTGTGACTTCGT |
| | | ACCAGAATCACAAGATCACACAGCTGAAAATTGAGAACAACCCCTTCGCCAAAGGCTTT |
| | | CGGGGCAGTGATGACCTGGAGTTACACAGGATGTCTCGGATGCAAAGTAAAGAGTATCC |
| | | TGTGGTTCCCAGGAGCACAGTGAGGCACAAAGTCACCTCCAACCACAGCCCCTTCAGCA |
| | | GCGAGACCCGAGCTCTCTCCACCTCATCCAATTTAGGGTCCCAGTACCAGTGTGAGAAT |
| | | GGTGTCTCTGGCCCCTCCCAGGACCTTCTGCCCCCACCTAACCCATACCCACTGGCCCA |
| | | GGAGCACAGCCAAATTTACCACTGTACCAAGAGGAAAGATGAGGAATGTTCCAGCACGG |
| | | AGCACCCCTATAAGAAGCCGTACATGGAGACATCCCCCAGCGAGGAAGACACCTTCTAT |
| | | CGCTCGGGCTACCCCCAGCAGCAGGGCCTGAGTACCTCTTACAGGACAGAGTCGGCCCA |
| | | GCGGCAGGCCTGCATGTATGCCAGCTCCGCTCCCCCCAGCGAGCCCGTGCCTAGCCTGG |
| | | AGGACATCAGCTGTAACACATGGCCCAGCATGCCCTCCTATAGCAGCTGTACCGTCACC |
| | | ACCGTGCAGCCCATGGACCGTCTTCCCTACCAGCACTTCTCCGCTCATTTCACCTCGGG |
| | | GCCCCTGGTCCCTCGGTTGGCTGGCATGGCCAACCATGGTTCTCCCCAGCTCGGCGAAG |
| | | GGATGTTTCAGCACCAGACCTCAGTGGCCCATCAGCCTGTGGTCAGGCAGTGCGGGCCT |
| | | CAGACTGGCCTTCAGTCTCCGGGCGGCCTCCAGCCCCCAGAGTTTCTCTACACTCACGG |
| | | CGTGCCCAGGACCCTGTCCCCCCATCAGTATCACTCGGTACACGGCGTCGGCATGGTGC |
| | | CAGAGTGGAGTGAGAATAGCTGA |
| Mouse | 4 | ATGAGTCTGGTGGGGGGCTTTCCCCACCACCCCGTGGTGCACCATGAGGGCTACCCATT |
| Hand2 | | CGCCGCAGCCGCCGCCGCCGCTGCCGCCGCCGCCGCCAGCCGCTGCAGCCACGAGGAGA |
| | | ACCCCTACTTCCACGGCTGGCTTATTGGCCACCCGGAGATGTCGCCCCCCGACTACAGT |
| | | ATGGCCCTGTCCTACAGCCCCGAGTACGCCAGCGGTGCCGCGGGCCTGGACCACTCCCA |
| | | TTATGGGGGAGTGCCGCCCGGCGCCGGGCCTCCCGGCCTGGGGGGGCCGCGCCCGGTGA |
| | | AGCGCCGGGGCACCGCCAACCGCAAGGAGCGGCGCAGGACTCAGAGCATCAACAGCGCC |
| | | TTCGCCGAGCTGCGCGAGTGCATCCCCAACGTGCCCGCCGACACCAAACTCTCCAAGAT |
| | | CAAGACACTGCGCCTGGCCACCAGCTACATCGCCTACCTCATGGATCTGCTGGCCAAGG |
| | | ACGACCAGAACGGAGAGGCGGAGGCCTTCAAGGCGGAGATCAAGAAGACCGACGTGAAA |
| | | GAGGAGAAGAGGAAGAAAGAGCTGAATGAGATCTTGAAAAGCACAGTGAGCAGCAACGA |
| | | CAAGAAAACCAAAGGCCGGACAGGCTGGCCACAGCACGTCTGGGCCCTGGAGCTCAAGC |
| | | AGTGA |
| Mouse | 5 | ATGTACCAAAGCCTGGCCATGGCCGCCAACCACGGCCCCCCGCCCGGCGCCTACGAAGC |
| Gata4-Flag | | AGGTGGCCCTGGCGCCTTCATGCACAGCGCGGGCGCCGCGTCCTCGCCCGTCTACGTGC |
| | | CCACTCCGCGGGTGCCGTCCTCTGTGCTGGGCCTGTCCTACCTGCAGGGCGGTGGCAGT |
| | | GCCGCTGCAGCTGGAACCACCTCGGGTGGCAGCTCCGGGGCCGGCCCGTCGGGTGCAGG |
| | | GCCTGGGACCCAGCAGGGTAGCCCTGGCTGGAGCCAAGCTGGAGCCGAGGGAGCCGCCT |
| | | ACACCCCGCCGCCCGTGTCCCCGCGCTTCTCTTTCCCGGGGACTACTGGGTCCCTGGCG |
| | | GCCGCTGCCGCCGCTGCCGCAGCCCGGGAAGCTGCAGCCTACGGCAGTGGCGGCGGGGC |
| | | GGCGGGCGCTGGTCTGGCTGGCCGAGAGCAGTACGGGCGTCCGGGCTTCGCCGGCTCCT |
| | | ACTCCAGCCCCTACCCAGCCTACATGGCCGACGTGGGAGCATCCTGGGCCGCAGCCGCT |
| | | GCCGCCTCTGCCGGCCCCTTCGACAGCCCAGTCCTGCACAGCCTGCCTGGACGGGCCAA |
| | | CCCTGGAAGACACCCCAATCTCGATATGTTTGATGACTTCTCAGAAGGCAGAGAGTGTG |
| | | TCAATTGTGGGGCCATGTCCACCCCACTCTGGAGGCGAGATGGGACGGGACACTACCTG |
| | | TGCAATGCCTGTGGCCTCTATCACAAGATGAACGGCATCAACCGGCCCCTCATTAAGCC |
| | | TCAGCGCCGCCTGTCCGCTTCCCGCCGGGTAGGCCTCTCCTGTGCCAACTGCCAGACTA |
| | | CCACCACCACGCTGTGGCGTCGTAATGCCGAGGGTGAGCCTGTATGTAATGCCTGCGGC |
| | | CTCTACATGAAGCTCCATGGGGTTCCCAGGCCTCTTGCAATGCGGAAGGAGGGGATTCA |
| | | AACCAGAAAACGGAAGCCCAAGAACCTGAATAAATCTAAGACGCCAGCAGGTCCTGCTG |
| | | GTGAGACCCTCCCTCCCTCCAGTGGTGCCTCCAGCGGTAACTCCAGCAATGCCACTAGC |
| | | AGCAGCAGCAGCAGTGAAGAGATGCGCCCCATCAAGACAGAGCCCGGGCTGTCATCTCA |
| | | CTATGGGCACAGCAGCTCCATGTCCCAGACATTCAGTACTGTGTCCGGCCACGGGCCCT |
| | | CCATCCATCCAGTGCTGTCTGCTCTGAAGCTGTCCCCACAAGGCTATGCATCTCCTGTC |
| | | ACTCAGACATCGCAGGCCAGCTCCAAGCAGGACTCTTGGAACAGCCTGGTCCTGGCTGA |
| | | CAGTCATGGGGACATAATCACCGCGGGAGGCGGTGGAGCCGACTACAAGGAC |
| | | CACGACGGCGACTACAAGGACCACGACATCGACTACAAGGACGACGA |
| | | CGACAAGGGGCCCGTT |
| Mouse | 6 | ATGGGGAGAAAAAAGATTCAGATTACGAGGATAATGGATGAGCGTAACAGACAGGTGAC |
| Mef2c- | | TTTTACGAAGAGGAAATTTGGATTGATGAAGAAGGCTTATGAGCTGAGCGTGCTGTGCG |
| Flag | | ACTGTGAGATTGCACTGATCATCTTCAACAGCACCAACAAGCTGTTCCAGTACGCCAGC |
| | | ACTGACATGGATAAGGTGTTGCTCAAGTACACCGAGTACAACGAGCCGCACGAGAGCCG |
| | | GACAAACTCAGACATTGTGGAGACATTGAGAAAGAAGGGCCTCAATGGCTGTGACAGCC |
| | | CAGATCCCGATGCAGACGATTCAGTAGGTCACAGCCCTGAGTCTGAGGACAAGTACAGG |
| | | AAAATTAACGAAGATATTGATCTAATGATCAGCAGGCAAAGATTGTGTGCTGTTCCACC |
| | | TCCCAGCTTTGAGATGCCAGTTACCATCCCAGTGTCCAGCCATAACAGTTTGGTGTACA |
| | | GCAATCCTGTCAGCACACTGGGAAACCCCAATCTTCTGCCACTGGCCCACCCGTCTCTG |
| | | CAGAGGAATAGTATGTCTCCTGGTGTAACACATAGACCTCCAAGTGCAGGTAACACAGG |
| | | CGGTCTGATGGGCGGAGATCTGACATCCGGTGCAGGCACCAGCGCAGGGAATGGATACG |
| | | GCAACCCCCGGAACTCACCAGGCCTGCTGGTCTCACCTGGTAACCTGAACAAGAATATA |
| | | CAAGCCAAATCTCCTCCCCCTATGAATCTAGGAATGAATAATCGTAAGCCAGATCTCCG |
| | | CGTTCTTATCCCACCTGGCAGCAAGAACACGATGCCATCAGTGAATCAAAGGATAAATA |
| | | ACTCCCAGTCGGCTCAGTCATTGGCTACCCCGGTGGTTTCCGTAGCAACTCCTACTTTA |
| | | CCAGGACAAGGAATGGGAGGATATCCATCAGCCATTTCAACAACATATGGTACTGAGTA |
| | | CTCTCTGAGTAGCGCAGATCTGTCATCTCTGTCTGGCTTCAACACTGCCAGTGCGCTCC |
| | | ACCTCGGCTCTGTAACTGGCTGGCAGCAGCAGCACCTACATAACATGCCGCCATCTGCC |
| | | CTCAGTCAGTTGGGAGCTTGCACTAGCACTCATTTATCTCAGAGTTCAAATCTCTCCCT |
| | | GCCTTCTACTCAAAGCCTCAGCATCAAGTCAGAACCTGTTTCTCCTCCTAGAGACCGTA |
| | | CCACCACCCCTTCGAGATACCCACAACACACCACGCGCCACGAGGCGGGGAGGTCTCCT |
| | | GTTGACAGCTTGAGCAGCTGTAGCAGTTCCTACGATGGGAGCGACCGAGAGGATCACCG |
| | | GAACGAATTCCACTCCCCCATTGGACTCACCAGACCTTCGCCGGACGAAAGGGAAAGTC |
| | | CTTCAGTCAAGCGCATGCGACTCTCTGAAGGATGGGCAACAGGAGGCGGTGGAGC |
| | | CGACTACAAGGACCACGACGGCGACTACAAGGACCACGACATCGACT |
| | | ACAAGGACGACGACGACAAGGGGCCCGTT |
| Mouse | 7 | ATGGCCGATACAGATGAGGGCTTTGGCCTGGCGCGCACGCCTCTGGAGCCTGATTCCAA |
| TBX5-Flag | | AGACAGGTCTTGCGATTCGAAACCTGAGAGTGCTCTGGGGGCTCCCAGCAAGTCTCCAT |
| | | CATCCCCGCAGGCTGCCTTCACCCAGCAGGGCATGGAAGGAATCAAGGTGTTTCTTCAT |
| | | GAACGTGAACTGTGGCTGAAGTTCCACGAAGTGGGCACAGAGATGATCATCACCAAGGC |
| | | AGGGAGGAGAATGTTTCCTAGTTACAAAGTGAAGGTGACTGGCCTTAATCCCAAAACGA |
| | | AGTATATTCTTCTCATGGATATTGTTCCCGCAGACGACCACAGATATAAATTTGCTGAT |
| | | AACAAATGGTCCGTAACTGGCAAAGCAGAGCCTGCCATGCCGGGGCGCCTTTACGTGCA |
| | | CCCGGACTCCCCAGCAACCGGAGCCCACTGGATGCGACAACTTGTCTCCTTCCAGAAGC |
| | | TCAAACTCACCAACAACCACCTGGACCCGTTTGGACACATTATCCTGAACTCCATGCAC |
| | | AAATACCAGCCCCGATTACACATCGTGAAAGCAGACGAAAATAATGGGTTCGGTTCAAA |
| | | GAACACTGCGTTTTGCACCCACGTCTTCCCGGAGACAGCTTTTATCGCTGTGACTTCGT |
| | | ACCAGAATCACAAGATCACACAGCTGAAAATTGAGAACAACCCCTTCGCCAAAGGCTTT |
| | | CGGGGCAGTGATGACCTGGAGTTACACAGGATGTCTCGGATGCAAAGTAAAGAGTATCC |
| | | TGTGGTTCCCAGGAGCACAGTGAGGCACAAAGTCACCTCCAACCACAGCCCCTTCAGCA |
| | | GCGAGACCCGAGCTCTCTCCACCTCATCCAATTTAGGGTCCCAGTACCAGTGTGAGAAT |
| | | GGTGTCTCTGGCCCCTCCCAGGACCTTCTGCCCCCACCTAACCCATACCCACTGGCCCA |
| | | GGAGCACAGCCAAATTTACCACTGTACCAAGAGGAAAGATGAGGAATGTTCCAGCACGG |
| | | AGCACCCCTATAAGAAGCCGTACATGGAGACATCCCCCAGCGAGGAAGACACCTTCTAT |
| | | CGCTCGGGCTACCCCCAGCAGCAGGGCCTGAGTACCTCTTACAGGACAGAGTCGGCCCA |
| | | GCGGCAGGCCTGCATGTATGCCAGCTCCGCTCCCCCCAGCGAGCCCGTGCCTAGCCTGG |
| | | AGGACATCAGCTGTAACACATGGCCCAGCATGCCCTCCTATAGCAGCTGTACCGTCACC |
| | | ACCGTGCAGCCCATGGACCGTCTTCCCTACCAGCACTTCTCCGCTCATTTCACCTCGGG |
| | | GCCCCTGGTCCCTCGGTTGGCTGGCATGGCCAACCATGGTTCTCCCCAGCTCGGCGAAG |
| | | GGATGTTTCAGCACCAGACCTCAGTGGCCCATCAGCCTGTGGTCAGGCAGTGCGGGCCT |
| | | CAGACTGGCCTTCAGTCTCCGGGCGGCCTCCAGCCCCCAGAGTTTCTCTACACTCACGG |
| | | CGTGCCCAGGACCCTGTCCCCCCATCAGTATCACTCGGTACACGGCGTCGGCATGGTGC |
| | | CAGAGTGGAGTGAGAATAGCGGAGGCGGTGGAGCCGACTACAAGGACCACG |
| | | ACGGCGACTACAAGGACCACGACATCGACTACAAGGACGACGACGAC |
| | | AAGGGGCCCGTT |
| ASAH1 | 8 | ATGCCGGGCCGGAGTTGCGTCGCCTTAGTCCTCCTGGCTGCCGCCGT |
| | | CAGCTGTGCCGTCGCGCAGCACGCGCCGCCGTGGACAGAGGACTGCA |
| | | GAAAATCAACCTATCCTCCTTCAGGACCAACGTACAGAGGTGCAGTT |
| | | CCATGGTACACCATAAATCTTGACTTACCACCCTACAAAAGATGGCA |
| | | TGAATTGATGCTTGACAAGGCACCAGTGCTAAAGGTTATAGTGAATT |
| | | CTCTGAAGAATATGATAAATACATTCGTGCCAAGTGGAAAAATTATG |
| | | CAGGTGGTGGATGAAAAATTGCCTGGCCTACTTGGCAACTTTCCTGG |
| | | CCCTTTTGAAGAGGAAATGAAGGGTATTGCCGCTGTTACTGATATAC |
| | | CTTTAGGAGAGATTATTTCATTCAATATTTTTTATGAATTATTTACC |
| | | ATTTGTACTTCAATAGTAGCAGAAGACAAAAAAGGTCATCTAATACA |
| | | TGGGAGAAACATGGATTTTGGAGTATTTCTTGGGTGGAACATAAATA |
| | | ATGATACCTGGGTCATAACTGAGCAACTAAAACCTTTAACAGTGAAT |
| | | TTGGATTTCCAAAGAAACAACAAAACTGTCTTCAAGGCTTCAAGCTT |
| | | TGCTGGCTATGTGGGCATGTTAACAGGATTCAAACCAGGACTGTTCA |
| | | GTCTTACACTGAATGAACGTTTCAGTATAAATGGTGGTTATCTGGGT |
| | | ATTCTAGAATGGATTCTGGGAAAGAAAGATGTCATGTGGATAGGGTT |
| | | CCTCACTAGAACAGTTCTGGAAAATAGCACAAGTTATGAAGAAGCCA |
| | | AGAATTTATTGACCAAGACCAAGATATTGGCCCCAGCCTACTTTATC |
| | | CTGGGAGGCAACCAGTCTGGGGAAGGTTGTGTGATTACACGAGACAG |
| | | AAAGGAATCATTGGATGTATATGAACTCGATGCTAAGCAGGGTAGAT |
| | | GGTATGTGGTACAAACAAATTATGACCGTTGGAAACATCCCTTCTTC |
| | | CTTGATGATCGCAGAACGCCTGCAAAGATGTGTCTGAACCGCACCAG |
| | | CCAAGAGAATATCTCATTTGAAACCATGTATGATGTCCTGTCAACAA |
| | | AACCTGTCCTCAACAAGCTGACCGTATACACAACCTTGATAGATGTT |
| | | ACCAAAGGTCAATTCGAAACTTACCTGCGGGACTGCCCTGACCCTTG |
| | | TATAGGTTGGTGA |
| Human | 9 | ATGTATCAGAGCTTGGCCATGGCCGCCAACCACGGGCCGCCCCCCGGTGCCTACGAGGC |
| GATA4 | | GGGCGGCCCCGGCGCCTTCATGCACGGCGCGGGCGCCGCGTCCTCGCCAGTCTACGTGC |
| | | CCACACCGCGGGTGCCCTCCTCCGTGCTGGGCCTGTCCTACCTCCAGGGCGGAGGCGCG |
| | | GGCTCTGCGTCCGGAGGCGCCTCGGGCGGCAGCTCCGGTGGGGCCGCGTCTGGTGCGGG |
| | | GCCCGGGACCCAGCAGGGCAGCCCGGGATGGAGCCAGGCGGGAGCCGACGGAGCCGCTT |
| | | ACACCCCGCCGCCGGTGTCGCCGCGCTTCTCCTTCCCGGGGACCACCGGGTCCCTGGCG |
| | | GCCGCCGCCGCCGCTGCCGCGGCCCGGGAAGCTGCGGCCTACAGCAGTGGCGGCGGAGC |
| | | GGCGGGTGCGGGCCTGGCGGGCCGCGAGCAGTACGGGCGCGCCGGCTTCGCGGGCTCCT |
| | | ACTCCAGCCCCTACCCGGCTTACATGGCCGACGTGGGCGCGTCCTGGGCCGCAGCCGCC |
| | | GCCGCCTCCGCCGGCCCCTTCGACAGCCCGGTCCTGCACAGCCTGCCCGGCCGGGCCAA |
| | | CCCGGCCGCCCGACACCCCAATCTCGTAGATATGTTTGACGACTTCTCAGAAGGCAGAG |
| | | AGTGTGTCAACTGTGGGGCTATGTCCACCCCGCTCTGGAGGCGAGATGGGACGGGTCAC |
| | | TATCTGTGCAACGCCTGCGGCCTCTACCACAAGATGAACGGCATCAACCGGCCGCTCAT |
| | | CAAGCCTCAGCGCCGGCTGTCCGCCTCCCGCCGAGTGGGCCTCTCCTGTGCCAACTGCC |
| | | AGACCACCACCACCACGCTGTGGCGCCGCAATGCGGAGGGCGAGCCTGTGTGCAATGCC |
| | | TGCGGCCTCTACATGAAGCTCCACGGGGTCCCCAGGCCTCTTGCAATGCGGAAAGAGGG |
| | | GATCCAAACCAGAAAACGGAAGCCCAAGAACCTGAATAAATCTAAGACACCAGCAGCTC |
| | | CTTCAGGCAGTGAGAGCCTTCCTCCCGCCAGCGGTGCTTCCAGCAACTCCAGCAACGCC |
| | | ACCACCAGCAGCAGCGAGGAGATGCGTCCCATCAAGACGGAGCCTGGCCTGTCATCTCA |
| | | CTACGGGCACAGCAGCTCCGTGTCCCAGACGTTCTCAGTCAGTGCGATGTCTGGCCATG |
| | | GGCCCTCCATCCACCCTGTCCTCTCGGCCCTGAAGCTCTCCCCACAAGGCTATGCGTCT |
| | | CCCGTCAGCCAGTCTCCACAGACCAGCTCCAAGCAGGACTCTTGGAACAGCCTGGTCTT |
| | | GGCCGACAGTCACGGGGACATAATCACTGCGTAA |
| Human | 10 | ATGGGGAGAAAAAAGATTCAGATTACGAGGATTATGGATGAACGTAACAGACAGGTGAC |
| Mef2c | | ATTTACAAAGAGGAAATTTGGGTTGATGAAGAAGGCTTATGAGCTGAGCGTGCTGTGTG |
| | | ACTGTGAGATTGCGCTGATCATCTTCAACAGCACCAACAAGCTGTTCCAGTATGCCAGC |
| | | ACCGACATGGACAAAGTGCTTCTCAAGTACACGGAGTACAACGAGCCGCATGAGAGCCG |
| | | GACAAACTCAGACATCGTGGAGGCATTGAACAAGAAAGAAAACAAAGGCTGTGAAAGCC |
| | | CCGATCCCGACTCCTCTTATGCACTCACCCCACGCACTGAAGAAAAATACAAAAAAATT |
| | | AATGAAGAATTTGATAATATGATCAAGAGTCATAAAATTCCTGCTGTTCCACCTCCCAA |
| | | CTTCGAGATGCCAGTCTCCATCCCAGTGTCCAGCCACAACAGTTTGGTGTACAGCAACC |
| | | CTGTCAGCTCACTGGGAAACCCCAACCTATTGCCACTGGCTCACCCTTCTCTGCAGAGG |
| | | AATAGTATGTCTCCTGGTGTAACACATCGACCTCCAAGTGCAGGTAACACAGGTGGTCT |
| | | GATGGGTGGAGACCTCACGTCTGGTGCAGGCACCAGTGCAGGGAACGGGTATGGCAATC |
| | | CCCGAAACTCACCAGGTCTGCTGGTCTCACCTGGTAACTTGAACAAGAATATGCAAGCA |
| | | AAATCTCCTCCCCCAATGAATTTAGGAATGAATAACCGTAAACCAGATCTCCGAGTTCT |
| | | TATTCCACCAGGCAGCAAGAATACGATGCCATCAGTGAATCAAAGGATAAATAACTCCC |
| | | AGTCGGCTCAGTCATTGGCTACCCCAGTGGTTTCCGTAGCAACTCCTACTTTACCAGGA |
| | | CAAGGAATGGGAGGATATCCATCAGCCATTTCAACAACATATGGTACCGAGTACTCTCT |
| | | GAGTAGTGCAGACCTGTCATCTCTGTCTGGGTTTAACACCGCCAGCGCTCTTCACCTTG |
| | | GTTCAGTAACTGGCTGGCAACAGCAACACCTACATAACATGCCACCA |
| Human | 11 | ATGGCCGACGCAGACGAGGGCTTTGGCCTGGCGCACACGCCTCTGGAGCCTGACGCAAA |
| Tbx5 | | AGACCTGCCCTGCGATTCGAAACCCGAGAGCGCGCTCGGGGCCCCCAGCAAGTCCCCGT |
| | | CGTCCCCGCAGGCCGCCTTCACCCAGCAGGGCATGGAGGGAATCAAAGTGTTTCTCCAT |
| | | GAAAGAGAACTGTGGCTAAAATTCCACGAAGTGGGCACGGAAATGATCATAACCAAGGC |
| | | TGGAAGGCGGATGTTTCCCAGTTACAAAGTGAAGGTGACGGGCCTTAATCCCAAAACGA |
| | | AGTACATTCTTCTCATGGACATTGTACCTGCCGACGATCACAGATACAAATTCGCAGAT |
| | | AATAAATGGTCTGTGACGGGCAAAGCTGAGCCCGCCATGCCTGGCCGCCTGTACGTGCA |
| | | CCCAGACTCCCCCGCCACCGGGGCGCATTGGATGAGGCAGCTCGTCTCCTTCCAGAAAC |
| | | TCAAGCTCACCAACAACCACCTGGACCCATTTGGGCATATTATTCTAAATTCCATGCAC |
| | | AAATACCAGCCTAGATTACACATCGTGAAAGCGGATGAAAATAATGGATTTGGCTCAAA |
| | | AAATACAGCGTTCTGCACTCACGTCTTTCCTGAGACTGCGTTTATAGCAGTGACTTCCT |
| | | ACCAGAACCACAAGATCACGCAATTAAAGATTGAGAATAATCCCTTTGCCAAAGGATTT |
| | | CGGGGCAGTGATGACATGGAGCTGCACAGAATGTCAAGAATGCAAAGTAAAGAATATCC |
| | | CGTGGTCCCCAGGAGCACCGTGAGGCAAAAAGTGGCCTCCAACCACAGTCCTTTCAGCA |
| | | GCGAGTCTCGAGCTCTCTCCACCTCATCCAATTTGGGGTCCCAATACCAGTGTGAGAAT |
| | | GGTGTTTCCGGCCCCTCCCAGGACCTCCTGCCTCCACCCAACCCATACCCACTGCCCCA |
| | | GGAGCATAGCCAAATTTACCATTGTACCAAGAGGAAAGAGGAAGAATGTTCCACCACAG |
| | | ACCATCCCTATAAGAAGCCCTACATGGAGACATCACCCAGTGAAGAAGATTCCTTCTAC |
| | | CGCTCTAGCTATCCACAGCAGCAGGGCCTGGGTGCCTCCTACAGGACAGAGTCGGCACA |
| | | GCGGCAAGCTTGCATGTATGCCAGCTCTGCGCCCCCCAGCGAGCCTGTGCCCAGCCTAG |
| | | AGGACATCAGCTGCAACACGTGGCCAAGCATGCCTTCCTACAGCAGCTGCACCGTCACC |
| | | ACCGTGCAGCCCATGGACAGGCTACCCTACCAGCACTTCTCCGCTCACTTCACCTCGGG |
| | | GCCCCTGGTCCCTCGGCTGGCTGGCATGGCCAACCATGGCTCCCCACAGCTGGGAGAGG |
| | | GAATGTTCCAGCACCAGACCTCCGTGGCCCACCAGCCTGTGGTCAGGCAGTGTGGGCCT |
| | | CAGACTGGCCTGCAGTCCCCTGGCACCCTTCAGCCCCCTGAGTTCCTCTACTCTCATGG |
| | | CGTGCCAAGGACTCTATCCCCTCATCAGTACCACTCTGTGCACGGAGTTGGCATGGTGC |
| | | CAGAGTGGAGCGACAATAGCTAA |
| Human | 12 | ATGAGTCTGGTAGGTGGTTTTCCCCACCACCCGGTGGTGCACCACGAGGGCTACCCGTT |
| Hand2 | | TGCCGCCGCCGCCGCCGCAGCTGCCGCCGCCGCCGCCAGCCGCTGCAGCCATGAGGAGA |
| | | ACCCCTACTTCCATGGCTGGCTCATCGGCCACCCCGAGATGTCGCCCCCCGACTACAGC |
| | | ATGGCCCTGTCCTACAGCCCCGAGTATGCCAGCGGCGCCGCCGGCCTGGACCACTCCCA |
| | | TTACGGGGGGGTGCCGCCGGGCGCCGGGCCCCCGGGCCTGGGGGGGCCGCGCCCGGTGA |
| | | AGCGCCGAGGCACCGCCAACCGCAAGGAGCGGCGCAGGACTCAGAGCATCAACAGCGCC |
| | | TTCGCCGAACTGCGCGAGTGCATCCCCAACGTACCCGCCGACACCAAACTCTCCAAAAT |
| | | CAAGACCCTGCGCCTGGCCACCAGCTACATCGCCTACCTCATGGACCTGCTGGCCAAGG |
| | | ACGACCAGAATGGCGAGGCGGAGGCCTTCAAGGCAGAGATCAAGAAGACCGACGTGAAA |
| | | GAGGAGAAGAGGAAGAAGGAGCTGAACGAAATCTTGAAAAGCACAGTGAGCAGCAACGA |
| | | CAAGAAAACCAAAGGCCGGACGGGCTGGCCGCAGCACGTCTGGGCCCTGGAGCTCAAGC |
| | | AGTGA |
| Human | 13 | ATGTATCAGAGCTTGGCCATGGCCGCCAACCACGGGCCGCCCCCCGGTGCCTACGAGGC |
| GATA4- | | GGGCGGCCCCGGCGCCTTCATGCACGGCGCGGGCGCCGCGTCCTCGCCAGTCTACGTGC |
| Flag | | CCACACCGCGGGTGCCCTCCTCCGTGCTGGGCCTGTCCTACCTCCAGGGCGGAGGCGCG |
| | | GGCTCTGCGTCCGGAGGCGCCTCGGGCGGCAGCTCCGGTGGGGCCGCGTCTGGTGCGGG |
| | | GCCCGGGACCCAGCAGGGCAGCCCGGGATGGAGCCAGGCGGGAGCCGACGGAGCCGCTT |
| | | ACACCCCGCCGCCGGTGTCGCCGCGCTTCTCCTTCCCGGGGACCACCGGGTCCCTGGCG |
| | | GCCGCCGCCGCCGCTGCCGCGGCCCGGGAAGCTGCGGCCTACAGCAGTGGCGGCGGAGC |
| | | GGCGGGTGCGGGCCTGGCGGGCCGCGAGCAGTACGGGCGCGCCGGCTTCGCGGGCTCCT |
| | | ACTCCAGCCCCTACCCGGCTTACATGGCCGACGTGGGCGCGTCCTGGGCCGCAGCCGCC |
| | | GCCGCCTCCGCCGGCCCCTTCGACAGCCCGGTCCTGCACAGCCTGCCCGGCCGGGCCAA |
| | | CCCGGCCGCCCGACACCCCAATCTCGTAGATATGTTTGACGACTTCTCAGAAGGCAGAG |
| | | AGTGTGTCAACTGTGGGGCTATGTCCACCCCGCTCTGGAGGCGAGATGGGACGGGTCAC |
| | | TATCTGTGCAACGCCTGCGGCCTCTACCACAAGATGAACGGCATCAACCGGCCGCTCAT |
| | | CAAGCCTCAGCGCCGGCTGTCCGCCTCCCGCCGAGTGGGCCTCTCCTGTGCCAACTGCC |
| | | AGACCACCACCACCACGCTGTGGCGCCGCAATGCGGAGGGCGAGCCTGTGTGCAATGCC |
| | | TGCGGCCTCTACATGAAGCTCCACGGGGTCCCCAGGCCTCTTGCAATGCGGAAAGAGGG |
| | | GATCCAAACCAGAAAACGGAAGCCCAAGAACCTGAATAAATCTAAGACACCAGCAGCTC |
| | | CTTCAGGCAGTGAGAGCCTTCCTCCCGCCAGCGGTGCTTCCAGCAACTCCAGCAACGCC |
| | | ACCACCAGCAGCAGCGAGGAGATGCGTCCCATCAAGACGGAGCCTGGCCTGTCATCTCA |
| | | CTACGGGCACAGCAGCTCCGTGTCCCAGACGTTCTCAGTCAGTGCGATGTCTGGCCATG |
| | | GGCCCTCCATCCACCCTGTCCTCTCGGCCCTGAAGCTCTCCCCACAAGGCTATGCGTCT |
| | | CCCGTCAGCCAGTCTCCACAGACCAGCTCCAAGCAGGACTCTTGGAACAGCCTGGTCTT |
| | | GGCCGACAGTCACGGGGACATAATCACTGCGTAAGGAGGCGGTGGAGCCGACTA |
| | | CAAGGACCACGACGGCGACTACAAGGACCACGACATCGACTACAAGG |
| | | ACGACGACGACAAGGGGCCCGTT |
| Human | 14 | ATGGGGAGAAAAAAGATTCAGATTACGAGGATTATGGATGAACGTAACAGACAGGTGAC |
| Mef2c- | | ATTTACAAAGAGGAAATTTGGGTTGATGAAGAAGGCTTATGAGCTGAGCGTGCTGTGTG |
| Flag | | ACTGTGAGATTGCGCTGATCATCTTCAACAGCACCAACAAGCTGTTCCAGTATGCCAGC |
| | | ACCGACATGGACAAAGTGCTTCTCAAGTACACGGAGTACAACGAGCCGCATGAGAGCCG |
| | | GACAAACTCAGACATCGTGGAGGCATTGAACAAGAAAGAAAACAAAGGCTGTGAAAGCC |
| | | CCGATCCCGACTCCTCTTATGCACTCACCCCACGCACTGAAGAAAAATACAAAAAAATT |
| | | AATGAAGAATTTGATAATATGATCAAGAGTCATAAAATTCCTGCTGTTCCACCTCCCAA |
| | | CTTCGAGATGCCAGTCTCCATCCCAGTGTCCAGCCACAACAGTTTGGTGTACAGCAACC |
| | | CTGTCAGCTCACTGGGAAACCCCAACCTATTGCCACTGGCTCACCCTTCTCTGCAGAGG |
| | | AATAGTATGTCTCCTGGTGTAACACATCGACCTCCAAGTGCAGGTAACACAGGTGGTCT |
| | | GATGGGTGGAGACCTCACGTCTGGTGCAGGCACCAGTGCAGGGAACGGGTATGGCAATC |
| | | CCCGAAACTCACCAGGTCTGCTGGTCTCACCTGGTAACTTGAACAAGAATATGCAAGCA |
| | | AAATCTCCTCCCCCAATGAATTTAGGAATGAATAACCGTAAACCAGATCTCCGAGTTCT |
| | | TATTCCACCAGGCAGCAAGAATACGATGCCATCAGTGAATCAAAGGATAAATAACTCCC |
| | | AGTCGGCTCAGTCATTGGCTACCCCAGTGGTTTCCGTAGCAACTCCTACTTTACCAGGA |
| | | CAAGGAATGGGAGGATATCCATCAGCCATTTCAACAACATATGGTACCGAGTACTCTCT |
| | | GAGTAGTGCAGACCTGTCATCTCTGTCTGGGTTTAACACCGCCAGCGCTCTTCACCTTG |
| | | GTTCAGTAACTGGCTGGCAACAGCAACACCTACATAACATGCCACCAGGAGGCGGTG |
| | | GAGCCGACTACAAGGACCACGACGGCGACTACAAGGACCACGACATC |
| | | GACTACAAGGACGACGACGACAAGGGGCCCGTT |
| Human | 15 | ATGGCCGACGCAGACGAGGGCTTTGGCCTGGCGCACACGCCTCTGGAGCCTGACGCAAA |
| Tbx5-Flag | | AGACCTGCCCTGCGATTCGAAACCCGAGAGCGCGCTCGGGGCCCCCAGCAAGTCCCCGT |
| | | CGTCCCCGCAGGCCGCCTTCACCCAGCAGGGCATGGAGGGAATCAAAGTGTTTCTCCAT |
| | | GAAAGAGAACTGTGGCTAAAATTCCACGAAGTGGGCACGGAAATGATCATAACCAAGGC |
| | | TGGAAGGCGGATGTTTCCCAGTTACAAAGTGAAGGTGACGGGCCTTAATCCCAAAACGA |
| | | AGTACATTCTTCTCATGGACATTGTACCTGCCGACGATCACAGATACAAATTCGCAGAT |
| | | AATAAATGGTCTGTGACGGGCAAAGCTGAGCCCGCCATGCCTGGCCGCCTGTACGTGCA |
| | | CCCAGACTCCCCCGCCACCGGGGCGCATTGGATGAGGCAGCTCGTCTCCTTCCAGAAAC |
| | | TCAAGCTCACCAACAACCACCTGGACCCATTTGGGCATATTATTCTAAATTCCATGCAC |
| | | AAATACCAGCCTAGATTACACATCGTGAAAGCGGATGAAAATAATGGATTTGGCTCAAA |
| | | AAATACAGCGTTCTGCACTCACGTCTTTCCTGAGACTGCGTTTATAGCAGTGACTTCCT |
| | | ACCAGAACCACAAGATCACGCAATTAAAGATTGAGAATAATCCCTTTGCCAAAGGATTT |
| | | CGGGGCAGTGATGACATGGAGCTGCACAGAATGTCAAGAATGCAAAGTAAAGAATATCC |
| | | CGTGGTCCCCAGGAGCACCGTGAGGCAAAAAGTGGCCTCCAACCACAGTCCTTTCAGCA |
| | | GCGAGTCTCGAGCTCTCTCCACCTCATCCAATTTGGGGTCCCAATACCAGTGTGAGAAT |
| | | GGTGTTTCCGGCCCCTCCCAGGACCTCCTGCCTCCACCCAACCCATACCCACTGCCCCA |
| | | GGAGCATAGCCAAATTTACCATTGTACCAAGAGGAAAGAGGAAGAATGTTCCACCACAG |
| | | ACCATCCCTATAAGAAGCCCTACATGGAGACATCACCCAGTGAAGAAGATTCCTTCTAC |
| | | CGCTCTAGCTATCCACAGCAGCAGGGCCTGGGTGCCTCCTACAGGACAGAGTCGGCACA |
| | | GCGGCAAGCTTGCATGTATGCCAGCTCTGCGCCCCCCAGCGAGCCTGTGCCCAGCCTAG |
| | | AGGACATCAGCTGCAACACGTGGCCAAGCATGCCTTCCTACAGCAGCTGCACCGTCACC |
| | | ACCGTGCAGCCCATGGACAGGCTACCCTACCAGCACTTCTCCGCTCACTTCACCTCGGG |
| | | GCCCCTGGTCCCTCGGCTGGCTGGCATGGCCAACCATGGCTCCCCACAGCTGGGAGAGG |
| | | GAATGTTCCAGCACCAGACCTCCGTGGCCCACCAGCCTGTGGTCAGGCAGTGTGGGCCT |
| | | CAGACTGGCCTGCAGTCCCCTGGCACCCTTCAGCCCCCTGAGTTCCTCTACTCTCATGG |
| | | CGTGCCAAGGACTCTATCCCCTCATCAGTACCACTCTGTGCACGGAGTTGGCATGGTGC |
| | | CAGAGTGGAGCGACAATAGCTAAGGAGGCGGTGGAGCCGACTACAAGGACCA |
| | | CGACGGCGACTACAAGGACCACGACATCGACTACAAGGACGACGACG |
| | | ACAAGGGGCCCGTT |
| ACVI | 16 | ATGCCGGGCCGGAGTTGCGTCGCCTTAGTCCTCCTGGCTGCCGCCGTCAGCTGTGCCGT |
| | | CGCGCAGCACGCGCCGCCGTGGACAGAGGACTGCAGAAAATCAACCTATCCTCCTTCAG |
| | | GACCAACGTACAGAGGTGCAGTTCCATGGTACACCATAAATCTTGACTTACCACCCTAC |
| | | AAAAGATGGCATGAATTGATGCTTGACAAGGCACCAGTGCTAAAGGTTATAGTGAATTC |
| | | TCTGAAGAATATGATAAATACATTCGTGCCAAGTGGAAAAATTATGCAGGTGGTGGATG |
| | | AAAAATTGCCTGGCCTACTTGGCAACTTTCCTGGCCCTTTTGAAGAGGAAATGAAGGGT |
| | | ATTGCCGCTGTTACTGATATACCTTTAGGAGAGATTATTTCATTCAATATTTTTTATGA |
| | | ATTATTTACCATTTGTACTTCAATAGTAGCAGAAGACAAAAAAGGTCATCTAATACATG |
| | | GGAGAAACATGGATTTTGGAGTATTTCTTGGGTGGAACATAAATAATGATACCTGGGTC |
| | | ATAACTGAGCAACTAAAACCTTTAACAGTGAATTTGGATTTCCAAAGAAACAACAAAAC |
| | | TGTCTTCAAGGCTTCAAGCTTTGCTGGCTATGTGGGCATGTTAACAGGATTCAAACCAG |
| | | GACTGTTCAGTCTTACACTGAATGAACGTTTCAGTATAAATGGTGGTTATCTGGGTATT |
| | | CTAGAATGGATTCTGGGAAAGAAAGATGTCATGTGGATAGGGTTCCTCACTAGAACAGT |
| | | TCTGGAAAATAGCACAAGTTATGAAGAAGCCAAGAATTTATTGACCAAGACCAAGATAT |
| | | TGGCCCCAGCCTACTTTATCCTGGGAGGCAACCAGTCTGGGGAAGGTTGTGTGATTACA |
| | | CGAGACAGAAAGGAATCATTGGATGTATATGAACTCGATGCTAAGCAGGGTAGATGGTA |
| | | TGTGGTACAAACAAATTATGACCGTTGGAAACATCCCTTCTTCCTTGATGATCGCAGAA |
| | | CGCCTGCAAAGATGTGTCTGAACCGCACCAGCCAAGAGAATATCTCATTTGAAACCATG |
| | | TATGATGTCCTGTCAACAAAACCTGTCCTCAACAAGCTGACCGTATACACAACCTTGAT |
| | | AGATGTTACCAAAGGTCAATTCGAAACTTACCTGCGGGACTGCCCTGACCCTTGTATAG |
| | | GTTGGTGA |
| Dn-TGFP | 17 | ATGGGTCGGGGGCTGCTCAGGGGCCTGTGGCCGCTGCACATCGTCCTGTGGACGCGTAT |
| | | CGCCAGCACGATCCCACCGCACGTTCAGAAGTCGGTTAATAACGACATGATAGTCACTG |
| | | ACAACAACGGTGCAGTCAAGTTTCCACAACTGTGTAAATTTTGTGATGTGAGATTTTCC |
| | | ACCTGTGACAACCAGAAATCCTGCATGAGCAACTGCAGCATCACCTCCATCTGTGAGAA |
| | | GCCACAGGAAGTCTGTGTGGCTGTATGGAGAAAGAATGACGAGAACATAACACTAGAGA |
| | | CAGTTTGCCATGACCCCAAGCTCCCCTACCATGACTTTATTCTGGAAGATGCTGCTTCT |
| | | CCAAAGTGCATTATGAAGGAAAAAAAAAAGCCTGGTGAGACTTTCTTCATGTGTTCCTG |
| | | TAGCTCTGATGAGTGCAATGACAACATCATCTTCTCAGAAGAATATAACACCAGCAATC |
| | | CTGACTTGTTGCTAGTCATATTTCAAGTGACAGGCATCAGCCTCCTGCCACCACTGGGA |
| | | GTTGCCATATCTGTCATCATCATCTTCTACTGCTACCGCGTTGGATCCATGGACTACAA |
| | | AGACGATGACGATAAATAG |
| CCN5 | 18 | ATGAGAGGCACACCGAAGACCCACCTCCTGGCCTTCTCCCTCCTCTGCCTCCTCTCAAA |
| | | GGTGCGTACCCAGCTGTGCCCGACACCATGTACCTGCCCCTGGCCACCTCCCCGATGCC |
| | | CGCTGGGAGTACCCCTGGTGCTGGATGGCTGTGGCTGCTGCCGGGTATGTGCACGGCGG |
| | | CTGGGGGAGCCCTGCGACCAACTCCACGTCTGCGACGCCAGCCAGGGCCTGGTCTGCCA |
| | | GCCCGGGGCAGGACCCGGTGGCCGGGGGGCCCTGTGCCTCTGTAAGCAGGACCCCAGTT |
| | | TTCTGGCCTTGTCTCTTCCCTGCCCCCTGGTGTCCCCTGCCCAGAATGGAGCACGGCCT |
| | | GGGGACCCTGCTCGACCACCTGTGGGCTGGGCATGGCCACCCGGGTGTCCAACCAGAAC |
| | | CGCTTCTGCCGACTGGAGACCCAGCGCCGCCTGTGCCTGTCCAGGCCCTGCCCACCCTC |
| | | CAGGGGTCGCAGTCCACAAAACAGTGCCTTCTAGAGCCGGGCTGGGAATGGGGACACGG |
| | | TGTCCACCATCCCCAGCTGGTGGCCCTGTGCCTGGGCCCTGGGCTGATGGAAGATGGTC |
| | | CGTGCCCAGGCCCTTGGCTGCAGGCAACACTTTAGCTTGGGTCCACCATGCAGAACACC |
| | | AATATTAA |
| dnWnt8 | 19 | ATGCAAAACACCACTTTGTTCATCCTTGCAACTCTTCTGATCTTCTGCCCATTCTTCAC |
| | | TGCCTCAGCATGGTCAGTCAATAACTTTCTGATGACAGGACCCAAGGCATATCTGACAT |
| | | ACTCAGCGAGTGTTGCCGTGGGTGCGCAGAATGGAATTGAGGAGTGTAAATATCAGTTT |
| | | GCGTGGGAAAGATGGAATTGCCCTGAAAGTACCCTGCAGCTTGCTACCCACAATGGACT |
| | | TCGAAGTGCAACCAGAGAAACCTCCTTTGTGCATGCCATTAGCTCAGCGGGAGTTATGT |
| | | ATACACTGACGAGAAACTGCAGCATGGGGGACTTTGATAACTGTGGATGTGATGACTCC |
| | | AGAAATGGCCGCATCGGTGGCCGAGGCTGGGTATGGGGCGGCTGCAGTGATAATGCAGA |
| | | ATTTGGTGAGCGGATCTCTAAACTATTCGTCGATGGCTTGGAGACGGGACAAGATGCCA |
| | | GAGCCCTAATGAACTTGCATAACAATGAAGCAGGAAGACTTGCAGTGAAAGAGACAATG |
| | | AAAAGGACTTGCAAGTGCCATGGAATATCTGGAAGTTGCAGCATACAAACTTGCTGGCT |
| | | TCAGCTGGCCGAGTTTCGGGATATTGGCAATCCCTTAAAGATCAAGCACGACCAAGCGC |
| | | TAAAGCTTGAGATGGACAANAGNAAATGA |
| WNT5a | 20 | ATGTCTTCCAAGTTCTTCCTAATGGCTTTGGCCACGTTTTTCTCCTTCGCCCAGGTTGT |
| | | TATAGAAGCTAATTCTTGGTGGTCTCTAGGTATGAATAACCCTGTTCAGATGTCAGAAG |
| | | TATATATCATAGGTGCACAGCCTCTCTGCAGCCAACTGGCAGGACTTTCTCAAGGACAG |
| | | AAGAAACTCTGCCACTTGTATCAGGACCACATGCAGTACATTGGAGAAGGTGCGAAGAC |
| | | AGGCATCAAGGAATGCCAGTACCAGTTCCGGCATCGGAGATGGAACTGCAGCACAGTGG |
| | | ACAATACTTCTGTCTTTGGCAGGGTGATGCAAATAGGCAGCCGAGAGACGGCCTTCACG |
| | | TACGCGGTGAGCGCAGCTGGGGTGGTGAACGCCATGAGCCGAGCATGCCGGGAGGGCGA |
| | | GCTGTCTACCTGTGGCTGCAGCCGCGCTGCGCGCCCCAAGGACCTGCCTCGGGACTGGT |
| | | TGTGGGGCGGCTGCGGAGACAACATCGACTATGGCTACCGCTTCGCCAAGGAGTTCGTG |
| | | GACGCTAGAGAAAGGGAACGAATCCACGCTAAGGGTTCCTATGAGAGCGCACGCATCCT |
| | | CATGAACTTACACAACAATGAAGCAGGCCGTAGGACAGTATACAACCTGGCAGATGTAG |
| | | CCTGTAAGTGTCATGGAGTGTCTGGCTCCTGTAGCCTCAAGACGTGCTGGCTGCAGCTG |
| | | GCGGACTTCCGGAAGGTGGGCGATGCCCTCAAGGAGAAGTATGATAGCGCGGCGGCCAT |
| | | GAGGCTCAACAGCCGGGGCAAGCTGGTGCAGGTCAACAGCCGCTTCAACTCCCCGACCA |
| | | CGCAGGACCTGGTCTACATCGACCCCAGTCCGGACTACTGTGTGCGCAACGAGAGCACT |
| | | GGCTCGCTGGGCACGCAGGGACGCCTGTGCAACAAGACCTCAGAGGGGATGGACGGCTG |
| | | CGAGCTCATGTGCTGTGGGCGTGGCTATGACCAGTTTAAGACAGTGCAGACCGAACGCT |
| | | GTCATTGCAAGTTTCACTGGTGCTGCTATGTCAAATGCAAGAAGTGCACGGAGATTGTG |
| | | GATCAGTTCGTGTGCAAATAG |
| SV40 | 21 | ATGCCTAGGCTTTTGCAAAAAGCTTTGCAAATATGGATAAAGTTTTAAACAGAGAGGAA |
| LargeT | | TCTTTGCAGCTAATGGACCTTCTAGGTCTTGAAAGGAGTGCCTGGGGGAATATTCCTCT |
| | | GATGAGAAAGGCATATTTAAAAAAATGCAAGGAGTTTCATCCTGATAAAGGAGGAGATG |
| | | AAGAAAAAATGAAGAAAATGAATACTCTGTACAAGAAAATGGAAGATGGAGTAAAATAT |
| | | GCTCATCAACCTGACTTTGGAGGCTTCTGGGATGCAACTGAGGTATTTGCTTCTTCCTT |
| | | AAATCCTGGTGTTGATGCAATGTACTGCAAACAATGGCCTGAGTGTGCAAAGAAAATGT |
| | | CTACTAACTGCATATGCTTGCTGTGCTTACTGAGGATGAAGCATGAAAATAGAAAATTA |
| | | TACAGGAAAGATCCACTTGTGTGGGTTGATTGCTACTGCTTCGATTGCTTTAGAATGTG |
| | | GTTTGGACTTGATCTTTGTGAAGGAACCTTACTTCTGTGGTGTGACATAATTGGACAAA |
| | | CTACCTACAGAGATTTAAAGCTATAAGGTAAATATAAAATTTTTAAGTGTATAATGTGT |
| | | TAAACTACTGATTCTAATTGTTTGTGTATTTTAGATTCCAACCTATGGAACTGATGAAT |
| | | GGGAGCAGTGGTGGAATGCCTTTAATGAGGAAAACCTGTTTTGCTCAGAAGAAATGCCA |
| | | TCTAGTGATGATGAGGCTACTGCTGACTCTCAACATTCTACTCCTCCAAAAAAGAAGAG |
| | | AAAGGTAGAAGACCCCAAGGACTTTCCTTCAGAATTGCTAAGTTTTTTGAGTCATGCTG |
| | | TGTTTAGTAATAGAACTCTTGCTTGCTTTGCTATTTACACCACAAAGGAAAAAGCTGCA |
| | | CTGCTATACAAGAAAATTATGGAAAAATAA |
| SRF | 22 | ATGATCACCAGTGAGACCGGCAAGGCACTGATTCAGACCTGCCTCAACTCGCCAGACTC |
| | | TCCACCCCGTTCAGACCCCACAACAGACCAGAGAATGAGTGCCACTGGCTTTGAAGAGA |
| | | CAGATCTCACCTACCAGGTGTCGGAGTCTGACAGCAGTGGGGAGACCAAGGACACACTG |
| | | AAGCCGGCGTTCACAGTCACCAACCTGCCGGGTACAACCTCCACCATCCAAACAGCACC |
| | | TAGCACCTCTACCACCATGCAAGTCAGCAGCGGCCCCTCCTTTCCCATCACCAACTACC |
| | | TGGCACCAGTGTCTGCTAGTGTCAGCCCCAGTGCTGTCAGCAGTGCCAATGGGACTGTG |
| | | CTGAAGAGTACAGGCAGCGGCCCTGTCTCCTCTGGGGGCCTTATGCAGCTGCCTACCAG |
| | | CTTCACCCTCATGCCTGGTGGGGCAGTGGCCCAGCAGGTCCCAGTGCAGGCCATTCAAG |
| | | TGCACCAGGCCCCACAGCAAGCGTCTCCCTCCCGTGACAGCAGCACAGACCTCACGCAG |
| | | ACCTCCTCCAGCGGGACAGTGACGCTGCCCGCCACCATCATGACGTCATCCGTGCCCAC |
| | | AACTGTGGGTGGCCACATGATGTACCCTAGCCCGCATGCGGTGATGTATGCCCCCACCT |
| | | CGGGCCTGGGTGATGGCAGCCTCACCGTGCTGAATGCCTTCTCCCAGGCACCATCCACC |
| | | ATGCAGGTGTCACACAGCCAGGTCCAGGAGCCAGGTGGCGTCCCCCAGGTGTTCCTGAC |
| | | AGCATCATCTGGGACAGTGCAGATCCCTGTTTCAGCAGTTCAGCTCCACCAGATGGCTG |
| | | TGATAGGGCAGCAGGCCGGGAGCAGCAGCAACCTCACCGAGCTACAGGTGGTGAACCTG |
| | | GACACCGCCCACAGCACCAAGAGTGAATGA |
| Human | 23 | ATGACACTCCTGGGGTCTGAGCATTCCTTGCTGATTAGGAGCAAGTT |
| Myocardin | | CAGATCAGTTTTACAGTTAAGACTTCAACAAAGAAGGACCCAGGAAC |
| | | AACTGGCTAACCAAGGCATAATACCACCACTGAAACGTCCAGCTGAA |
| | | TTCCATGAGCAAAGAAAACATTTGGATAGTGACAAGGCTAAAAATTC |
| | | CCTGAAGCGCAAAGCCAGAAACAGGTGCAACAGTGCCGACTTGGTTA |
| | | ATATGCACATACTCCAAGCTTCCACTGCAGAGAGGTCCATTCCAACT |
| | | GCTCAGATGAAGCTGAAAAGAGCCCGACTCGCCGATGATCTCAATGA |
| | | AAAAATTGCTCTACGACCAGGGCCACTGGAGCTGGTGGAAAAAAACA |
| | | TTCTTCCTGTGGATTCTGCTGTGAAAGAGGCCATAAAAGGTAACCAG |
| | | GTGAGTTTCTCCAAATCCACGGATGCTTTTGCCTTTGAAGAGGACAG |
| | | CAGCAGCGATGGGCTTTCTCCGGATCAGACTCGAAGTGAAGACCCCC |
| | | AAAACTCAGCGGGATCCCCGCCAGACGCTAAAGCCTCAGATACCCCT |
| | | TCGACAGGTTCTCTGGGGACAAACCAGGATCTTGCTTCTGGCTCAGA |
| | | AAATGACAGAAATGACTCAGCCTCACAGCCCAGCCACCAGTCAGATG |
| | | CGGGGAAGCAGGGGCTTGGCCCCCCCAGCACCCCCATAGCCGTGCAT |
| | | GCTGCTGTAAAGTCCAAATCCTTGGGTGACAGTAAGAACCGCCACAA |
| | | AAAGCCCAAGGACCCCAAGCCAAAGGTGAAGAAGCTTAAATATCACC |
| | | AGTACATTCCCCCAGACCAGAAGGCAGAGAAGTCCCCTCCACCTATG |
| | | GACTCAGCCTACGCTCGGCTGCTCCAGCAACAGCAGCTGTTCCTGCA |
| | | GCTCCAAATCCTCAGCCAGCAGCAGCAGCAGCAGCAACACCGATTCA |
| | | GCTACCTAGGGATGCACCAAGCTCAGCTTAAGGAACCAAATGAACAG |
| | | ATGGTCAGAAATCCAAACTCTTCTTCAACGCCACTGAGCAATACCCC |
| | | CTTGTCTCCTGTCAAAAACAGTTTTTCTGGACAAACTGGTGTCTCTT |
| | | CTTTCAAACCAGGCCCACTCCCACCTAACCTGGATGATCTGAAGGTC |
| | | TCTGAATTAAGACAACAGCTTCGAATTCGGGGCTTGCCTGTGTCAGG |
| | | CACCAAAACGGCTCTCATGGACCGGCTTCGACCCTTCCAGGACTGCT |
| | | CTGGCAACCCAGTGCCGAACTTTGGGGATATAACGACTGTCACTTTT |
| | | CCTGTCACACCCAACACGCTGCCCAATTACCAGTCTTCCTCTTCTAC |
| | | CAGTGCCCTGTCCAACGGCTTCTACCACTTTGGCAGCACCAGCTCCA |
| | | GCCCCCCGATCTCCCCAGCCTCCTCTGACCTGTCAGTCGCTGGGTCC |
| | | CTGCCGGACACCTTCAATGATGCCTCCCCCTCCTTCGGCCTGCACCC |
| | | GTCCCCAGTCCACGTGTGCACGGAGGAAAGTCTCATGAGCAGCCTGA |
| | | ATGGGGGCTCTGTTCCTTCTGAGCTGGATGGGCTGGACTCCGAGAAG |
| | | GACAAGATGCTGGTGGAGAAGCAGAAGGTGATCAATGAACTCACCTG |
| | | GAAACTCCAGCAAGAGCAGAGGCAGGTGGAGGAGCTGAGGATGCAGC |
| | | TTCAGAAGCAGAAAAGGAATAACTGTTCAGAGAAGAAGCCGCTGCCT |
| | | TTCCTGGCTGCCTCCATCAAGCAGGAAGAGGCTGTCTCCAGCTGTCC |
| | | TTTTGCATCCCAAGTACCTGTGAAAAGACAAAGCAGCAGCTCAGAGT |
| | | GTCACCCACCGGCTTGTGAAGCTGCTCAACTCCAGCCTCTTGGAAAT |
| | | GCTCATTGTGTGGAGTCCTCAGATCAAACCAATGTACTTTCTTCCAC |
| | | ATTTCTCAGCCCCCAGTGTTCCCCTCAGCATTCACCGCTGGGGGCTG |
| | | TGAAAAGCCCACAGCACATCAGTTTGCCCCCATCACCCAACAACCCT |
| | | CACTTTCTGCCCTCATCCTCCGGGGCCCAGGGAGAAGGGCACAGGGT |
| | | CTCCTCGCCCATCAGCAGCCAGGTGTGCACTGCACAGAACTCAGGAG |
| | | CACACGATGGCCATCCTCCAAGCTTCTCTCCCCATTCTTCCAGCCTC |
| | | CACCCGCCCTTCTCTGGAGCCCAAGCAGACAGCAGTCATGGTGCCGG |
| | | GGGAAACCCTTGTCCCAAAAGCCCATGTGTACAGCAAAAGATGGCTG |
| | | GTTTACACTCTTCTGATAAGGTGGGGCCAAAGTTTTCAATTCCATCC |
| | | CCAACTTTTTCTAAGTCAAGTTCAGCAATTTCAGAGGTAACACAGCC |
| | | TCCATCCTATGAAGATGCCGTAAAGCAGCAAATGACCCGGAGTCAGC |
| | | AGATGGATGAACTCCTGGACGTGCTTATTGAAAGCGGAGAAATGCCA |
| | | GCAGACGCTAGAGAGGATCACTCATGTCTTCAAAAAGTCCCAAAGAT |
| | | ACCCAGATCTTCCCGAAGTCCAACTGCTGTCCTCACCAAGCCCTCGG |
| | | CTTCCTTTGAACAAGCCTCTTCAGGCAGCCAGATCCCCTTTGATCCC |
| | | TATGCCACCGACAGTGATGAGCATCTTGAAGTCTTATTAAATTCCCA |
| | | GAGCCCCCTAGGAAAGATGAGTGATGTCACCCTTCTAAAAATTGGGA |
| | | GCGAAGAGCCTCACTTTGATGGGATAATGGATGGATTCTCTGGGAAG |
| | | GCTGCAGAAGACCTCTTCAATGCACATGAGATCTTGCCAGGCCCCCT |
| | | CTCTCCAATGCAGACACAGTTTTCACCCTCTTCTGTGGACAGCAATG |
| | | GGCTGCAGTTAAGCTTCACTGAATCTCCCTGGGAAACCATGGAGTGG |
| | | CTGGACCTCACTCCGCCAAATTCCACACCAGGCTTTAGCGCCCTCAC |
| | | CACCAGCAGCCCCAGCATCTTCAACATCGATTTCCTGGATGTCACTG |
| | | ATCTCAATTTGAATTCTTCCATGGACCTTCACTTGCAGCAGTGGTAG |
| ETS1 | 24 | ATGAGCTACTTTGTGGATTCTGCTGGGAGCAGCCCCGTCCCTTACTCAGCGCCTCGTCC |
| | | TGCAGTGGTGAGGCAAGGACCTAGCAACACTTATGAAGATCCTCGAATGAACTGTGGTT |
| | | TCCAGTCCAATTATCACCAGCAAAGACCTTGCTACCCCTTTTGGGATGAGATGGCAACT |
| | | CAGGAAGTTCCTACTGGTCTTGAACACTGTGTCTCAGATATGGAATGTGCAGATGTCCC |
| | | ACTATTAACTCCAAGCAGCAAAGAAATGATGTCTCAAGCATTAAAAGCTACTTTCAGTG |
| | | GTTTCACTAAAGAACAGCAACGACTGGGGATCCCAAAAGACCCCCGGCAGTGGACAGAA |
| | | ACCCATGTTCGGGACTGGGTGATGTGGGCTGTGAATGAATTCAGCCTGAAAGGTGTAGA |
| | | CTTCCAGAAGTTCTGTATGAATGGAGCAGCCCTCTGCGCCCTGGGTAAAGACTGCTTTC |
| | | TCGAGCTGGCCCCAGACTTTGTTGGGGACATCTTATGGGAACATCTAGAGATCCTGCAG |
| | | AAAGAGGATGTGAAACCATATCAAGTTAATGGAGTCAACCCAGCCTATCCAGAATCCCG |
| | | CTATACCTCGGATTACTTCATTAGCTATGGTATTGAGCATGCCCAGTGTGTTCCACCAT |
| | | CGGAGTTCTCAGAGCCCAGCTTCATCACAGAGTCCTATCAGACGCTCCATCCCATCAGC |
| | | TCGGAAGAGCTCCTCTCCCTCAAGTATGAGAATGACTACCCCTCGGTCATTCTCCGAGA |
| | | CCCTCTCCAGACAGACACCTTGCAGAATGACTACTTTGCTATCAAACAAGAAGTCGTCA |
| | | CCCCAGACAACATGTGCATGGGGAGGACCAGTCGTGGTAAACTCGGGGGCCAGGACTCT |
| | | TTTGAAAGCATAGAGAGCTACGATAGTTGTGATCGCCTCACCCAGTCCTGGAGCAGCCA |
| | | GTCATCTTTCAACAGCCTGCAGCGTGTTCCCTCCTATGACAGCTTCGACTCAGAGGACT |
| | | ATCCGGCTGCCCTGCCCAACCACAAGCCCAAGGGCACCTTCAAGGACTATGTGCGGGAC |
| | | CGTGCTGACCTCAATAAGGACAAGCCTGTCATTCCTGCTGCTGCCCTAGCTGGCTACAC |
| | | AGGCAGTGGACCAATCCAGCTATGGCAGTTTCTTCTGGAATTACTCACTGATAAATCCT |
| | | GTCAGTCTTTTATCAGCTGGACAGGAGATGGCTGGGAATTCAAACTTTCTGACCCAGAT |
| | | GAGGTGGCCAGGAGATGGGGAAAGAGGAAAAACAAACCTAAGATGAATTATGAGAAACT |
| | | GAGCCGTGGCCTACGCTACTATTACGACAAAAACATCATCCACAAGACAGCGGGGAAAC |
| | | GCTACGTGTACCGCTTTGTGTGTGACCTGCAGAGCCTGCTGGGGTACACCCCTGAGGAG |
| | | CTGCACGCCATGCTGGACGTCAAGCCAGATGCCGACGAGTGA |
| ETS2 | 25 | ATGGGGTCGGCTCAATTTCAGGGCCTTATTACCCAAGCCCGGCTGCCCTTCGGTGCCAC |
| | | CAGCACCACTGCTCCGTCGCTGCGGAATTCCAAAGGCAGGTTTGGCGTTAGGGCCTTGG |
| | | CCCCAGAGAGGACGCCGAGCGCTCCACGGAAAGTCTCCGCCCGGCTCCCAGGGCGCACA |
| | | CTCGCGCGCACGTGGGGCCGAGGCCCTGCTCCCGGGGCCTCAGGGCCAGCCGGCGAGGG |
| | | ACCCAGCCGAGTGACAGCAGGAGGCGGAGGGAAGCTCAGAGCTCCCGGAGCCGCCCGGC |
| | | CAGCGTCCGGCCTCCCTGATCGTCTCTGGCCGGCGCCCTCGCCCTCGCCCGGCGCGCAC |
| | | CGAGCAGCCGCGGGCGCCGAGCAGCCACCGTCCCGACCAAGCGCCGGCCCTGCCCGCAG |
| | | CGGCAGGATGAATGATTTCGGAATCAAGAATATGGACCAGGTAGCCCCTGTGGCTAACA |
| | | GTTACAGAGGGACACTCAAGCGCCAGCCAGCCTTTGACACCTTTGATGGGTCCCTGTTT |
| | | GCTGTTTTTCCTTCTCTAAATGAAGAGCAAACACTGCAAGAAGTGCCAACAGGCTTGGA |
| | | TTCCATTTCTCATGACTCCGCCAACTGTGAATTGCCTTTGTTAACCCCGTGCAGCAAGG |
| | | CTGTGATGAGTCAAGCCTTAAAAGCTACCTTCAGTGGCTTCAAAAAGGAACAGCGGCGC |
| | | CTGGGCATTCCAAAGAACCCCTGGCTGTGGAGTGAGCAACAGGTATGCCAGTGGCTTCT |
| | | CTGGGCCACCAATGAGTTCAGTCTGGTGAACGTGAATCTGCAGAGGTTCGGCATGAATG |
| | | GCCAGATGCTGTGTAACCTTGGCAAGGAACGCTTTCTGGAGCTGGCACCTGACTTTGTG |
| | | GGTGACATTCTCTGGGAACATCTGGAGCAAATGATCAAAGAAAACCAAGAAAAGACAGA |
| | | AGATCAATATGAAGAAAATTCACACCTCACCTCCGTTCCTCATTGGATTAACAGCAATA |
| | | CATTAGGTTTTGGCACAGAGCAGGCGCCCTATGGAATGCAGACACAGAATTACCCCAAA |
| | | GGCGGCCTCCTGGACAGCATGTGTCCGGCCTCCACACCCAGCGTACTCAGCTCTGAGCA |
| | | GGAGTTTCAGATGTTCCCCAAGTCTCGGCTCAGCTCCGTCAGCGTCACCTACTGCTCTG |
| | | TCAGTCAGGACTTCCCAGGCAGCAACTTGAATTTGCTCACCAACAATTCTGGGACTCCC |
| | | AAAGACCACGACTCCCCTGAGAACGGTGCGGACAGCTTCGAGAGCTCAGACTCCCTCCT |
| | | CCAGTCCTGGAACAGCCAGTCGTCCTTGCTGGATGTGCAACGGGTTCCTTCCTTCGAGA |
| | | GCTTCGAAGATGACTGCAGCCAGTCTCTCTGCCTCAATAAGCCAACCATGTCTTTCAAG |
| | | GATTACATCCAAGAGAGGAGTGACCCAGTGGAGCAAGGCAAACCAGTTATACCTGCAGC |
| | | TGTGCTGGCCGGCTTCACAGGAAGTGGACCTATTCAGCTGTGGCAGTTTCTCCTGGAGC |
| | | TGCTATCAGACAAATCCTGCCAGTCATTCATCAGCTGGACTGGAGACGGATGGGAGTTT |
| | | AAGCTCGCCGACCCCGATGAGGTGGCCCGCCGGTGGGGAAAGAGGAAAAATAAGCCCAA |
| | | GATGAACTACGAGAAGCTGAGCCGGGGCTTACGCTACTATTACGACAAGAACATCATCC |
| | | ACAAGACGTCGGGGAAGCGCTACGTGTACCGCTTCGTGTGCGACCTCCAGAACTTGCTG |
| | | GGGTTCACGCCCGAGGAACTGCACGCCATCCTGGGCGTCCAGCCCGACACGGAGGACTG |
| | | A |
| ESRRG | 26 | ATGTCAAACAAAGATCGACACATTGATTCCAGCTGTTCGTCCTTCATCAAGACGGAACC |
| | | TTCCAGCCCAGCCTCCCTGACGGACAGCGTCAACCACCACAGCCCTGGTGGCTCTTCAG |
| | | ACGCCAGTGGGAGCTACAGTTCAACCATGAATGGCCATCAGAACGGACTTGACTCGCCA |
| | | CCTCTCTACCCTTCTGCTCCTATCCTGGGAGGTAGTGGGCCTGTCAGGAAACTGTATGA |
| | | TGACTGCTCCAGCACCATTGTTGAAGATCCCCAGACCAAGTGTGAATACATGCTCAACT |
| | | CGATGCCCAAGAGACTGTGTTTAGTGTGTGGTGACATCGCTTCTGGGTACCACTATGGG |
| | | GTAGCATCATGTGAAGCCTGCAAGGCATTCTTCAAGAGGACAATTCAAGGCAATATAGA |
| | | ATACAGCTGCCCTGCCACGAATGAATGTGAAATCACAAAGCGCAGACGTAAATCCTGCC |
| | | AGGCTTGCCGCTTCATGAAGTGTTTAAAAGTGGGCATGCTGAAAGAAGGGGTGCGTCTT |
| | | GACAGAGTACGTGGAGGTCGGCAGAAGTACAAGCGCAGGATAGATGCGGAGAACAGCCC |
| | | ATACCTGAACCCTCAGCTGGTTCAGCCAGCCAAAAAGCCATATAACAAGATTGTCTCAC |
| | | ATTTGTTGGTGGCTGAACCGGAGAAGATCTATGCCATGCCTGACCCTACTGTCCCCGAC |
| | | AGTGACATCAAAGCCCTCACTACACTGTGTGACTTGGCCGACCGAGAGTTGGTGGTTAT |
| | | CATTGGATGGGCGAAGCATATTCCAGGCTTCTCCACGCTGTCCCTGGCGGACCAGATGA |
| | | GCCTTCTGCAGAGTGCTTGGATGGAAATTTTGATCCTTGGTGTCGTATACCGGTCTCTT |
| | | TCGTTTGAGGATGAACTTGTCTATGCAGACGATTATATAATGGACGAAGACCAGTCCAA |
| | | ATTAGCAGGCCTTCTTGATCTAAATAATGCTATCCTGCAGCTGGTAAAGAAATACAAGA |
| | | GCATGAAGCTGGAAAAAGAAGAATTTGTCACCCTCAAAGCTATAGCTCTTGCTAATTCA |
| | | GACTCCATGCACATAGAAGATGTTGAAGCCGTTCAGAAGCTTCAGGATGTCTTACATGA |
| | | AGCGCTGCAGGATTATGAAGCTGGCCAGCACATGGAAGACCCTCGTCGAGCTGGCAAGA |
| | | TGCTGATGACACTGCCACTCCTGAGGCAGACCTCTACCAAGGCCGTGCAGCATTTCTAC |
| | | AACATCAAACTAGAAGGCAAAGTCCCAATGCACAAACTTTTTTTGGAAATGTTGGAGGC |
| | | CAAGGTCTGA |
| SMARCD3 | 27 | ATGGCCGCGGACGAAGTTGCCGGAGGGGCGCGCAAAGCCACGAAAAGCAAACTTTTTGA |
| | | GTTTCTGGTCCATGGGGTGCGCCCCGGGATGCCGTCTGGAGCCCGGATGCCCCACCAGG |
| | | GGGCGCCCATGGGCCCCCCGGGCTCCCCGTACATGGGCAGCCCCGCCGTGCGACCCGGC |
| | | CTGGCCCCCGCGGGCATGGAGCCCGCCCGCAAGCGAGCAGCGCCCCCGCCCGGGCAGAG |
| | | CCAGGCACAGAGCCAGGGCCAGCCGGTGCCCACCGCCCCCGCGCGGAGCCGCAGTGCCA |
| | | AGAGGAGGAAGATGGCTGACAAAATCCTCCCTCAAAGGATTCGGGAGCTGGTCCCCGAG |
| | | TCCCAGGCTTACATGGACCTCTTGGCATTTGAGAGGAAACTGGATCAAACCATCATGCG |
| | | GAAGCGGGTGGACATCCAGGAGGCTCTGAAGAGGCCCATGAAGCAAAAGCGGAAGCTGC |
| | | GACTCTATATCTCCAACACTTTTAACCCTGCGAAGCCTGATGCTGAGGATTCCGACGGC |
| | | AGCATTGCCTCCTGGGAGCTACGGGTGGAGGGGAAGCTCCTGGATGATCCCAGCAAACA |
| | | GAAGCGGAAGTTCTCTTCTTTCTTCAAGAGTTTGGTCATCGAGCTGGACAAAGATCTTT |
| | | ATGGCCCTGACAACCACCTCGTTGAGTGGCATCGGACACCCACGACCCAGGAGACGGAC |
| | | GGCTTCCAGGTGAAACGGCCTGGGGACCTGAGTGTGCGCTGCACGCTGCTCCTCATGCT |
| | | GGACTACCAGCCTCCCCAGTTCAAACTGGATCCCCGCCTAGCCCGGCTGCTGGGGCTGC |
| | | ACACACAGAGCCGCTCAGCCATTGTCCAGGCCCTGTGGCAGTATGTGAAGACCAACAGG |
| | | CTGCAGGACTCCCATGACAAGGAATACATCAATGGGGACAAGTATTTCCAGCAGATTTT |
| | | TGATTGTCCCCGGCTGAAGTTTTCTGAGATTCCCCAGCGCCTCACAGCCCTGCTATTGC |
| | | CCCCTGACCCAATTGTCATCAACCATGTCATCAGCGTGGACCCTTCAGACCAGAAGAAG |
| | | ACGGCGTGCTATGACATTGACGTGGAGGTGGAGGAGCCATTAAAGGGGCAGATGAGCAG |
| | | CTTCCTCCTATCCACGGCCAACCAGCAGGAGATCAGTGCTCTGGACAGTAAGATCCATG |
| | | AGACGATTGAGTCCATAAACCAGCTCAAGATCCAGAGGGACTTCATGCTAAGCTTCTCC |
| | | AGAGACCCCAAAGGCTATGTCCAAGACCTGCTCCGCTCCCAGAGCCGGGACCTCAAGGT |
| | | GATGACAGATGTAGCCGGCAACCCTGAAGAGGAGCGCCGGGCTGAGTTCTACCACCAGC |
| | | CCTGGTCCCAGGAGGCCGTCAGTCGCTACTTCTACTGCAAGATCCAGCAGCGCAGGCAG |
| | | GAGCTGGAGCAGTCGCTGGTTGTGCGCAACACCTAG |
| ZFPM2 | 28 | ATGTCCCGGCGAAAGCAAAGCAAACCCCGGCAGATCAAACGTGATGATGAAGGAATCCA |
| | | GGAGACAGCAGAATCAGATGGGGACACACAGTCAGAGAAACCGGGGCAACCTGGAGTTG |
| | | AGACAGACGACTGGGATGGACCAGGAGAGCTGGAGGTGTTTCAGAAAGATGGGGAACGA |
| | | AAAATTCAGAGTCGACAGCAACTTCCAGTGGGAACAACCTGGGGGCCGTTTCCTGGGAA |
| | | GATGGACTTGAATAATAATTCTTTGAAGACAAAGGCTCAGGTCCCAATGGTGCTGACTG |
| | | CTGGTCCCAAGTGGTTGCTGGATGTGACTTGGCAAGGAGTGGAAGACAACAAAAACAAC |
| | | TGCATTGTGTACAGCAAAGGGGGTCAGCTTTGGTGTACAACTACGAAGGCCATCTCTGA |
| | | GGGTGAAGAGCTAATTGCCTTTGTGGTGGATTTTGACTCAAGGCTACAAGCTGCCAGTC |
| | | AGATGACTCTCACAGAAGGGATGTACCCTGCACGCCTGCTGGACTCAATTCAGCTGCTT |
| | | CCTCAGCAAGCTGCCATGGCTTCTATTTTGCCCACAGCTATTGTCAATAAGGATATATT |
| | | CCCTTGCAAGTCCTGTGGCATCTGGTATCGGAGTGAGCGGAATCTGCAGGCCCATTTGA |
| | | TGTACTACTGCAGTGGGAGGCAAAGAGAAGCTGCTCCGGTGTCAGAGGAAAATGAAGAC |
| | | AGTGCCCATCAGATTTCCAGCCTGTGCCCCTTCCCACAGTGCACCAAGAGCTTTTCAAA |
| | | TGCTCGAGCTCTAGAAATGCACCTGAATTCACACAGTGGAGTGAAAATGGAAGAATTCC |
| | | TGCCCCCTGGTGCTAGTCTAAAATGCACCGTCTGTAGCTACACTGCTGATTCCGTGATC |
| | | AACTTTCACCAACACCTGTTCTCCCATCTCACTCAAGCTGCCTTCCGATGTAATCACTG |
| | | CCATTTCGGCTTCCAGACTCAGAGGGAGTTATTGCAGCACCAGGAGCTCCATGTCCCTA |
| | | GCGGCAAACTTCCCAGAGAAAGTGACATGGAACACTCTCCAAGTGCAACTGAAGACAGC |
| | | TTACAGCCAGCCACAGACTTATTGACCAGAAGCGAACTTCCCCAGAGCCAAAAGGCCAT |
| | | GCAGACTAAAGATGCGAGCTCTGACACAGAGCTGGACAAGTGTGAGAAAAAGACTCAGC |
| | | TCTTTCTCACGAACCAGAGACCAGAGATACAGCCTACAACAAATAAACAAAGCTTTTCT |
| | | TACACAAAAATAAAGTCTGAGCCCTCTAGCCCAAGACTTGCCTCATCTCCAGTTCAGCC |
| | | TAATATTGGGCCTTCTTTCCCTGTGGGCCCTTTCCTATCTCAGTTTTCTTTCCCCCAAG |
| | | ATATCACCATGGTCCCTCAAGCTTCAGAGATCTTAGCTAAGATGTCTGAACTGGTGCAT |
| | | CGGCGACTGAGGCATGGCAGTAGTAGCTACCCTCCCGTCATTTACAGCCCTTTGATGCC |
| | | CAAGGGGGCTACTTGTTTTGAGTGTAACATAACATTCAATAATTTGGATAATTATCTAG |
| | | TGCACAAAAAGCATTATTGCAGCAGCCGATGGCAGCAGATGGCTAAGTCCCCAGAGTTC |
| | | CCTAGTGTGTCAGAAAAGATGCCTGAAGCTTTGAGTCCCAACACTGGCCAAACCTCCAT |
| | | AAACCTTCTCAACCCAGCTGCTCATTCTGCTGATCCTGAGAATCCACTTCTTCAAACAT |
| | | CTTGCATCAATTCTTCCACTGTCTTAGATTTAATTGGGCCAAATGGGAAGGGCCATGAC |
| | | AAGGACTTTTCCACTCAAACTAAGAAGCTCTCCACCTCCAGTAACAATGATGACAAAAT |
| | | TAATGGAAAACCTGTTGATGTGAAAAATCCCAGTGTCCCCTTAGTGGATGGGGAAAGTG |
| | | ACCCAAATAAGACTACCTGTGAAGCTTGCAACATTACCTTCAGCCGGCACGAAACATAC |
| | | ATGGTCCACAAACAGTATTACTGTGCTACACGCCACGACCCTCCACTGAAGAGGTCTGC |
| | | TTCCAACAAAGTGCCTGCCATGCAGAGAACCATGCGCACACGCAAGCGCAGAAAGATGT |
| | | ATGAGATGTGCCTACCTGAGCAGGAACAAAGGCCTCCACTGGTTCAGCAGAGATTTCTT |
| | | GACGTAGCCAACCTCAATAATCCTTGTACCTCCACTCAAGAACCCACAGAAGGGCTAGG |
| | | AGAGTGCTACCACCCAAGATGTGATATCTTTCCAGGAATTGTCTCTAAACACTTGGAAA |
| | | CTTCTCTGACGATCAACAAGTGTGTTCCAGTTTCCAAATGTGATACTACTCATTCCAGT |
| | | GTTTCCTGCCTAGAGATGGACGTGCCCATAGATCTCAGCAAAAAGTGTTTATCTCAGTC |
| | | TGAGCGGACGACCACGTCTCCCAAAAGGCTGCTGGACTATCACGAGTGCACTGTGTGCA |
| | | AGATCAGTTTCAATAAGGTAGAAAACTATCTGGCCCACAAGCAGAATTTCTGCCCGGTT |
| | | ACTGCACATCAGCGTAATGACCTGGGTCAACTGGACGGCAAAGTGTTTCCGAATCCAGA |
| | | AAGCGAACGAAACAGCCCTGATGTCAGCTACGAAAGAAGCATAATAAAATGTGAGAAAA |
| | | ATGGGAATTTGAAGCAGCCTTCCCCCAATGGAAACTTATTTTCATCCCACCTAGCAACC |
| | | CTGCAAGGCTTGAAGGTCTTTAGTGAAGCTGCTCAGCTCATTGCTACAAAAGAAGAAAA |
| | | CAGACATTTGTTTCTTCCACAATGCCTTTACCCTGGAGCAATAAAGAAAGCAAAAGGAG |
| | | CCGACCAGCTTTCTCCATATTATGGAATCAAGCCAAGTGATTATATTTCTGGTTCTCTT |
| | | GTCATCCATAACACTGACATCGAGCAAAGCAGAAATGCAGAAAATGAATCTCCTAAAGG |
| | | CCAGGCTTCCTCAAATGGGTGTGCTGCGCTGAAGAAAGATTCTCTGCCATTGTTGCCCA |
| | | AAAATCGAGGAATGGTAATAGTGAATGGTGGACTGAAACAAGATGAGAGACCTGCTGCC |
| | | AACCCACAGCAAGAGAACATTTCCCAGAATCCTCAGCACGAAGACGACCACAAATCTCC |
| | | CTCGTGGATCTCTGAGAACCCATTAGCTGCCAATGAGAATGTCTCACCAGGAATTCCCT |
| | | CAGCAGAGGAACAGTTGTCTAGTATAGCAAAAGGTGTGAATGGTTCCAGCCAGGCTCCA |
| | | ACCAGTGGGAAATATTGCCGGCTATGTGATATCCAGTTCAACAACCTTTCAAACTTTAT |
| | | AACTCACAAGAAGTTTTATTGCTCATCACATGCAGCAGAACATGTCAAATGA |
| DN SNAI1 | 29 | CACTATAGGGCGAATTGGCGGAAGGCCGTCAAGGCCGCATTTGGACCCTCGTACAGAAG |
| | | CTAATACGACTCACTATAGGGAAATAAGAGAGAAAATAAGAGTAAGAAGAAATATAAGA |
| | | GCCACCATGAGGAAGCCGTCCGACCCCCGCCGGAAGCCCAACTATAGCGAGCTGCAGGA |
| | | CGCGTGTGTGGAGTTCACCTTCCAGCAGCCCTACGACCAGGCCCACCTGCTGGCCGCCA |
| | | TCCCTCCGCCCGAGGTCCTCAACCCCGCCGCTTCGCTGCCCACCCTCATCTGGGACTCT |
| | | CTCCTGGTACCCCAAGTGCGGCCGGTTGCCTGGGCCACCCTCCCGCTGCGGGAGAGCCC |
| | | CAAGGCCGTAGAGCTGACCTCGCTGTCCGATGAGGACAGTGGCAAAAGCTCCCAGCCGC |
| | | CCAGCCCGCCCTCGCCGGCGCCGTCGTCCTTCTCGTCCACCTCGGCCTCCTCCCTGGAG |
| | | GCCGAGGCCTTCATCGCCTTCCCTGGCTTGGGCCAACTTCCCAAGCAGCTGGCCAGGCT |
| | | CTCGGTGGCCAAGGACCCCCAGTCGCGGAAGATCTTCAACTGCAAATATTGTAACAAGG |
| | | AGTACCTCAGCCTGGGCGCTCTGAAGATGCACATCCGAAGCCACACGCTGCCTTGTGTC |
| | | TGCACGACCTGTGGAAAGGCCTTCTCTAGGCCCTGGCTGCTTCAGGGCCACGTCCGCAC |
| | | CCACACTGGTGAGAAGCCATTCTCCTGCTCCCACTGCAACCGTGCTTTTGCTGACCGCT |
| | | CCAACCTGCGTGCCCACCTCCAAACCCACTCGGATGTGAAGAGATACCAGTGCCAGGCC |
| | | TGTGCCCGAACCTTCTCCCGCATGTCCTTGCTCCACAAGCACCAAGAGTCTGGCTGCTC |
| | | CGGAGGCCCTCGCTGAGCTGCCTTCTGCGGGGCTTGCCTTCTGGCCATGCCCTTCTTCT |
| | | CTCCCTTGCACCTGTACCTCTTGGTCTTTGAATAAAGCCTGAGTAGGAACTGGGCCTCA |
| | | TGGGCCTTCCGCTCACTGCCCGCTTTCCAG |
| Mesp1 | 30 | ATGGCCCAGCCCCTGTGCGAGCCGCGCTCCGAGTCCTGGATCCTGAGTCCCGCTGGTCG |
| | | GCAGCCACCGATGCCTTCCGATGGGAACAGCGTCTGCTCCCCAGCCTGGTCCTCGGACC |
| | | CGTGGGACGGTGCCCAGGCCAGCAGCCCTGCACCACCCTGCGCCCGCCCGGCCCGGCGT |
| | | GCTGGGACCCCGGGTAGGCGCGGGACGCACGGTAGCCGCCTGGGTAGCGGACAGCGGCA |
| | | GAGCGCCAGCGAGCGGGAGAAGCTACGTATGCGCACACTCGCCCGCGCGCTGCACGAGC |
| | | TGCGCCGCTTCTTGCCGCCATCCGTGGCACCAACCGGCCAGAACCTGACCAAGATCGAG |
| | | ACGCTGCGCCTGGCCATCCGCTACATTGGCCACCTGTCGGCTGTGCTGGGACTCAGCGA |
| | | GGACAACCTCCGGCGACAGCGGCACGCGGTGTCACCTCGAGGCTGCCCGCTGTGCCCCG |
| | | ACAGCGACCTGGCGCAGTCGCAGTCGCTCGGTCCCCGTTTAAGCCCGGCCGTCTGCAGC |
| | | GGGGTGTCGTGGGGATCCCCGCCTGCCTACCCTAGACCCCGAGTCGCCGCAGAATCGTG |
| | | GGACCCATCGTTCCTGTACGCAGAAACAGCATCCCAGGAAAGGCAGGAAATGGAGCCCA |
| | | GTCCCTCATCTCCGCTCTTCAGCAGCGACATGCTGGCTCTTCTAGAAACCTGGACGCCG |
| | | CCGCAGGAGTGGCCGCCTGCCTGA |
| Sumo1 | 31 | ATGTCTGACCAGGAGGCAAAACCTTCAACTGAGGACTTAGGCGATAAGAAGGAAGGAGA |
| | | ATACATTAAACTCAAAGTTATTGGACAGGATAGCAGTGAGATACATTTCAAAGTGAAAA |
| | | TGACAACACATCTCAAGAAACTCAAAGAATCATACTGTCAAAGACAGGGAGTTCCAATG |
| | | AATTCACTCAGGTTTCTCTTTGAAGGTCAGAGAATTGCTGATAATCATACTCCGAAAGA |
| | | ACTGGGAATGGAGGAAGAAGATGTGATTGAAGTTTATCAGGAACAAACGGGGGGTCACT |
| | | CGACGGTTTAG |
| NKX2.5 | 32 | ATGTTCCCCAGCCCTGCGCTCACACCCACGCCTTTCTCAGTCAAAGACATCCTGAACCT |
| | | GGAGCAGCAGCAGCGTAGCCTGGCGTCTGGGGACCTGTCTGCGCGCCTCGAGGCCACCC |
| | | TGGCCCCTGCCTCCTGCATGCTGGCCGCCTTCAAGCCCGAGGCCTACTCTGGCCCCGAG |
| | | GCGGCAGCGTCCGGCCTGGCAGAGCTGCGCGCGGAGATGGGCCCCGCGCCTTCGCCCCC |
| | | CAAGTGCTCTCCTGCTTTCCCAGCCGCCCCCACATTTTACCCGGGAGCCTACGGTGACC |
| | | CTGACCCAGCCAAAGACCCTCGGGCGGATAAAAAAGAGCTGTGCGCGCTGCAGAAGGCA |
| | | GTGGAGCTGGACAAAGCCGAGACGGATGGCGCCGAGAGACCACGCGCACGGCGGCGACG |
| | | GAAGCCACGCGTGCTCTTCTCGCAGGCGCAGGTCTACGAGCTGGAGCGGCGCTTCAAGC |
| | | AACAGCGGTACCTGTCGGCGCCAGAGCGCGACCAGCTGGCCAGCGTGCTGAAGCTCACG |
| | | TCCACGCAGGTCAAGATCTGGTTCCAGAACCGTCGCTACAAGTGCAAGCGACAGCGGCA |
| | | GGACCAGACTCTGGAGCTTCTGGGGCCGCCGCCGCCGCCCGCGCGCAGGATCGCGGTGC |
| | | CCGTGCTGGTGCGCGACGGGAAGCCCTGCCTGGGGGACCCCGCGGCCTACGCTCCCGCC |
| | | TACGGCGTGGGTCTCAATGCCTATGGCTACAACGCCTACCCCTACCCCAGCTACGGCGG |
| | | CGCGGCCTGCAGTCCCGGCTACAGCTGCGCCGCCTACCCCGCTGCGCCCCCCGCCGCGC |
| | | AGCCCCCCGCCGCCTCCGCCAACAGCAACTTCGTGAACTTTGGCGTCGGGGACTTGAAC |
| | | ACCGTGCAGAGTCCCGGGATGCCGCAGGGCAATTCGGGCGTCTCCACGCTGCACGGCAT |
| | | CCGAGCCTGGTAG |
| Tbx6 | 33 | ATGTACCATCCACGAGAGTTGTACCCCTCCCTGGGGACTGGCTACCGTCTGGGACACCC |
| | | CCAGCCTGGGGCAGACTCCACCTTCCCACCTGCCCTGACAGAGGGTTACCGCTACCCTG |
| | | ATTTGGATACTTCTAAACTGGATTGCTTCCTCTCTGGGATCGAGGCAGCTCCCCACACT |
| | | CTGGCTGCAGCCGCTCCTTTGCCCCTTCTCCCATCTGCTCTGGGCCCCGAGACAGCACC |
| | | GCCACCCCCAGAGGCCCTTCACTCGCTTCCTGGGGTCAGCCTGAGCTTGGAGAACCAGG |
| | | AACTGTGGAAGGAATTCAGCGCTGTGGGGACAGAGATGATCATCACCAAGGCTGGCAGG |
| | | CGCATGTTCCCTGCTTGCCGAGTATCAGTCACTGGCCTGGACCCAGAGGCCCGCTACTT |
| | | GTTTCTTCTGGATGTGGTTCCAGTGGATGGGGCCCGATACCGCTGGCAGGGCCAGCACT |
| | | GGGAGCCAAGTGGCAAGGCTGAACCCCGCCTACCCGACCGTGTCTACATTCACCCTGAC |
| | | TCTCCTGCCACTGGTGCCCACTGGATGCGGCAGCCCGTATCCTTCCATCGTGTTAAGCT |
| | | CACCAACAGCACACTGGACCCCCATGGCCACCTGATCTTGCACTCGATGCACAAGTACC |
| | | AGCCTCGCATCCACCTGGTGAGAGCCACCCAACTATGCAGCCAACACTGGGGGGGTGTG |
| | | GCCTCCTTCCGATTTCCTGAGACCACATTCATCTCTGTGACAGCCTACCAGAACCCTAG |
| | | GATCACACAGCTGAAGATCGCAGCCAATCCCTTTGCCAAAGGTTTCCGAGAAAATGGCA |
| | | GAAACTGTAAGAGGGAGCGGGATGCCCGTGTGAAGAGGAAACTTCGGGGCCCAGAGCCA |
| | | GTGGCCACAGAGGCCTGTGGGAGTGGGGATACACCAGGGGGTCCCTGTGACTCCACCCT |
| | | GGGTGGGGACATTCGGGACTCAGATCCAGAGCAGGCCCCAACCCCCCAGGAAGCTGCTT |
| | | CTGCCTCAGCTCCTCCATGTGGGGGCCCCAGTGCTGAGGCCTACCTTCTACACCCTGCC |
| | | GCTTTTCATGGCGCCCCCAGTCACCTACCAGCCAGGACCCCCAGCTTCGCTGAGGCTCC |
| | | AGACCCTGGGCGCCCAGCCCCCTACTCAGCTGCATTTCTGGACCTACAGCCTGGACCAG |
| | | GGGGCTCTGCCTATCAGGCAGCTCCATCTGTACCATCCTTTGCCCCACACTTCATCCAA |
| | | GGGGGTCCCTTCCCTCTACCGTACCCAGGACCTGGAGGTTATCTGGACATGGGATCCAA |
| | | GCCAATGTACTGA |
| Ad52E4 | 34 | ATGGCTGATCAACATATATATGTGCATTTGCTGGGACGTCGGGCCTTTTTGCCCCAGCA |
| | | ACAGGGTTATTCTAATATGTATGTTCTTTTTTCACCGGAGGATTTTGTGCTTGCTCCCA |
| | | GAGGGATTATTTTGCTGTCTTTGCAGCTGTCGTTGGATATTCCCACTGGGTATCTGGGA |
| | | CGTTTTTTTTCTGTGGCGGATATGAATGTGAGGGGGGTATTGCTGTGTGCTCAGGAGAT |
| | | CCAACCGAGTACATGGTGGGAAGTTTCTGTTGTTCTTTTTAATCATTCGGATGAATTTT |
| | | ACCGCGGTTCCCGGGGACAGCCGGTTGCTTGCCTGCTGCTGGAGCGTGTCATATATCCC |
| | | ACCGTTCGCCAAGCTTCTTTAGTTTAA |
| Human | 35 | ATGCCGCGCGCTCCCCGCTGCCGAGCCGTGCGCTCCCTGCTGCGCAGCCACTACCGCGA |
| TERT | | GGTGCTGCCGCTGGCCACGTTCGTGCGGCGCCTGGGGCCCCAGGGCTGGCGGCTGGTGC |
| | | AGCGCGGGGACCCGGCGGCTTTCCGCGCGCTGGTGGCCCAGTGCCTGGTGTGCGTGCCC |
| | | TGGGACGCACGGCCGCCCCCCGCCGCCCCCTCCTTCCGCCAGGTGTCCTGCCTGAAGGA |
| | | GCTGGTGGCCCGAGTGCTGCAGAGGCTGTGCGAGCGCGGCGCGAAGAACGTGCTGGCCT |
| | | TCGGCTTCGCGCTGCTGGACGGGGCCCGCGGGGGCCCCCCCGAGGCCTTCACCACCAGC |
| | | GTGCGCAGCTACCTGCCCAACACGGTGACCGACGCACTGCGGGGGAGCGGGGCGTGGGG |
| | | GCTGCTGCTGCGCCGCGTGGGCGACGACGTGCTGGTTCACCTGCTGGCACGCTGCGCGC |
| | | TCTTTGTGCTGGTGGCTCCCAGCTGCGCCTACCAGGTGTGCGGGCCGCCGCTGTACCAG |
| | | CTCGGCGCTGCCACTCAGGCCCGGCCCCCGCCACACGCTAGTGGACCCCGAAGGCGTCT |
| | | GGGATGCGAACGGGCCTGGAACCATAGCGTCAGGGAGGCCGGGGTCCCCCTGGGCCTGC |
| | | CAGCCCCGGGTGCGAGGAGGCGCGGGGGCAGTGCCAGCCGAAGTCTGCCGTTGCCCAAG |
| | | AGGCCCAGGCGTGGCGCTGCCCCTGAGCCGGAGCGGACGCCCGTTGGGCAGGGGTCCTG |
| | | GGCCCACCCGGGCAGGACGCGTGGACCGAGTGACCGTGGTTTCTGTGTGGTGTCACCTG |
| | | CCAGACCCGCCGAAGAAGCCACCTCTTTGGAGGGTGCGCTCTCTGGCACGCGCCACTCC |
| | | CACCCATCCGTGGGCCGCCAGCACCACGCGGGCCCCCCATCCACATCGCGGCCACCACG |
| | | TCCCTGGGACACGCCTTGTCCCCCGGTGTACGCCGAGACCAAGCACTTCCTCTACTCCT |
| | | CAGGCGACAAGGAGCAGCTGCGGCCCTCCTTCCTACTCAGCTCTCTGAGGCCCAGCCTG |
| | | ACTGGCGCTCGGAGGCTCGTGGAGACCATCTTTCTGGGTTCCAGGCCCTGGATGCCAGG |
| | | GACTCCCCGCAGGTTGCCCCGCCTGCCCCAGCGCTACTGGCAAATGCGGCCCCTGTTTC |
| | | TGGAGCTGCTTGGGAACCACGCGCAGTGCCCCTACGGGGTGCTCCTCAAGACGCACTGC |
| | | CCGCTGCGAGCTGCGGTCACCCCAGCAGCCGGTGTCTGTGCCCGGGAGAAGCCCCAGGG |
| | | CTCTGTGGCGGCCCCCGAGGAGGAGGACACAGACCCCCGTCGCCTGGTGCAGCTGCTCC |
| | | GCCAGCACAGCAGCCCCTGGCAGGTGTACGGCTTCGTGCGGGCCTGCCTGCGCCGGCTG |
| | | GTGCCCCCAGGCCTCTGGGGCTCCAGGCACAACGAACGCCGCTTCCTCAGGAACACCAA |
| | | GAAGTTCATCTCCCTGGGGAAGCATGCCAAGCTCTCGCTGCAGGAGCTGACGTGGAAGA |
| | | TGAGCGTGCGGGACTGCGCTTGGCTGCGCAGGAGCCCAGGGGTTGGCTGTGTTCCGGCC |
| | | GCAGAGCACCGTCTGCGTGAGGAGATCCTGGCCAAGTTCCTGCACTGGCTGATGAGTGT |
| | | GTACGTCGTCGAGCTGCTCAGGTCTTTCTTTTATGTCACGGAGACCACGTTTCAAAAGA |
| | | ACAGGCTCTTTTTCTACCGGAAGAGTGTCTGGAGCAAGTTGCAAAGCATTGGAATCAGA |
| | | CAGCACTTGAAGAGGGTGCAGCTGCGGGAGCTGTCGGAAGCAGAGGTCAGGCAGCATCG |
| | | GGAAGCCAGGCCCGCCCTGCTGACGTCCAGACTCCGCTTCATCCCCAAGCCTGACGGGC |
| | | TGCGGCCGATTGTGAACATGGACTACGTCGTGGGAGCCAGAACGTTCCGCAGAGAAAAG |
| | | AGGGCCGAGCGTCTCACCTCGAGGGTGAAGGCACTGTTCAGCGTGCTCAACTACGAGCG |
| | | GGCGCGGCGCCCCGGCCTCCTGGGCGCCTCTGTGCTGGGCCTGGACGATATCCACAGGG |
| | | CCTGGCGCACCTTCGTGCTGCGTGTGCGGGCCCAGGACCCGCCGCCTGAGCTGTACTTT |
| | | GTCAAGGTGGATGTGACGGGCGCGTACGACACCATCCCCCAGGACAGGCTCACGGAGGT |
| | | CATCGCCAGCATCATCAAACCCCAGAACACGTACTGCGTGCGTCGGTATGCCGTGGTCC |
| | | AGAAGGCCGCCCATGGGCACGTCCGCAAGGCCTTCAAGAGCCACGTCTCTACCTTGACA |
| | | GACCTCCAGCCGTACATGCGACAGTTCGTGGCTCACCTGCAGGAGACCAGCCCGCTGAG |
| | | GGATGCCGTCGTCATCGAGCAGAGCTCCTCCCTGAATGAGGCCAGCAGTGGCCTCTTCG |
| | | ACGTCTTCCTACGCTTCATGTGCCACCACGCCGTGCGCATCAGGGGCAAGTCCTACGTC |
| | | CAGTGCCAGGGGATCCCGCAGGGCTCCATCCTCTCCACGCTGCTCTGCAGCCTGTGCTA |
| | | CGGCGACATGGAGAACAAGCTGTTTGCGGGGATTCGGCGGGACGGGCTGCTCCTGCGTT |
| | | TGGTGGATGATTTCTTGTTGGTGACACCTCACCTCACCCACGCGAAAACCTTCCTCAGC |
| | | TATGCCCGGACCTCCATCAGAGCCAGTCTCACCTTCAACCGCGGCTTCAAGGCTGGGAG |
| | | GAACATGCGTCGCAAACTCTTTGGGGTCTTGCGGCTGAAGTGTCACAGCCTGTTTCTGG |
| | | ATTTGCAGGTGAACAGCCTCCAGACGGTGTGCACCAACATCTACAAGATCCTCCTGCTG |
| | | CAGGCGTACAGGTTTCACGCATGTGTGCTGCAGCTCCCATTTCATCAGCAAGTTTGGAA |
| | | GAACCCCACATTTTTCCTGCGCGTCATCTCTGACACGGCCTCCCTCTGCTACTCCATCC |
| | | TGAAAGCCAAGAACGCAGGGATGTCGCTGGGGGCCAAGGGCGCCGCCGGCCCTCTGCCC |
| | | TCCGAGGCCGTGCAGTGGCTGTGCCACCAAGCATTCCTGCTCAAGCTGACTCGACACCG |
| | | TGTCACCTACGTGCCACTCCTGGGGTCACTCAGGACAGCCCAGACGCAGCTGAGTCGGA |
| | | AGCTCCCGGGGACGACGCTGACTGCCCTGGAGGCCGCAGCCAACCCGGCACTGCCCTCA |
| | | GACTTCAAGACCATCCTGGACTGA |
| Pkm2 | 36 | ATGACTCAACCTTTCAACTTTGCGGCTTTTGAGAAAATTTTCTTCCTTGGCTCTTCAGG |
| | | GCAAAGCATACTCCACTCTGAGCGATCTTTCCAAGTGAGGCCACCAAGCCACACTGAGT |
| | | CAACAAAACAGCAATATGCCATCAGAAGCCCAGACCCAGAGAACCAAAGGACTTCAGGA |
| | | ACCATGCCGAAGCCACACAGTGAAGCAGGGACTGCCTTCATTCAGACCCAGCAGCTCCA |
| | | TGCAGCCATGGCTGACACCTTCCTGGAACACATGTGCCGCCTGGACATTGACTCTGCCC |
| | | CCATCACGGCCCGCAACACTGGCATCATTTGTACCATTGGGCCTGCTTCCCGATCTGTG |
| | | GAGATGCTGAAGGAGATGATTAAGTCTGGAATGAATGTGGCTCGGCTGAATTTCTCTCA |
| | | TGGAACCCATGAGTACCATGCAGAGACCATCAAGAATGTCCGTGAAGCCACAGAAAGCT |
| | | TTGCATCTGATCCCATTCTCTACCGTCCTGTTGCGGTGGCTCTGGATACAAAGGGACCT |
| | | GAGATCCGGACTGGACTCATCAAGGGCAGCGGCACCGCTGAGGTGGAGCTGAAGAAGGG |
| | | AGCCACTCTGAAGATCACCCTGGACAACGCTTACATGGAGAAGTGTGACGAGAACATCC |
| | | TGTGGCTGGACTACAAGAACATCTGCAAGGTGGTGGAGGTGGGCAGCAAGATCTACGTG |
| | | GACGATGGGCTCATCTCACTGCAGGTGAAGGAGAAAGGCGCTGACTTCCTGGTGACGGA |
| | | GGTGGAGAATGGTGGCTCCTTGGGCAGCAAGAAGGGCGTGAACCTGCCGGGCGCTGCTG |
| | | TGGATCTCCCCGCTGTGTCGGAAAAGGACATCCAGGACCTGAAGTTTGGGGTGGAGCAG |
| | | GATGTGGACATGGTGTTTGCATCTTTCATCCGCAAGGCAGCCGACGTGCATGAAGTCAG |
| | | GAAGGTGCTGGGAGAGAAGGGCAAGAACATCAAGATCATCAGCAAAATCGAGAACCATG |
| | | AAGGCGTCCGCAGGTTTGATGAGATCTTGGAGGCCAGTGATGGGATCATGGTGGCTCGT |
| | | GGTGACCTGGGCATTGAGATTCCTGCAGAGAAGGTCTTCCTGGCTCAGAAGATGATGAT |
| | | CGGGCGATGCAACCGAGCTGGGAAGCCTGTCATCTGTGCCACACAGATGCTGGAGAGCA |
| | | TGATCAAGAAGCCACGCCCCACCCGTGCTGAAGGCAGTGATGTGGCCAATGCAGTCCTG |
| | | GATGGAGCAGACTGCATCATGCTGTCTGGAGAAACAGCCAAGGGGGACTACCCTCTGGA |
| | | GGCTGTTCGCATGCAGCACCTGATAGCTCGGGAGGCTGAGGCAGCCATGTTCCACCGTC |
| | | TGCTGTTTGAAGAGCTTGTGCGAGCCTCCAGTCACTCCACAGACCTCATGGAGGCCATG |
| | | GCCATGGGCAGCGTGGAGGCCTCTTATAAGTGTTTAGCAGCAGCTTTGATAGTTCTCAC |
| | | GGAGTCTGGCAGGAGTGCTCACCAAGTGGCCAGGTACCGCCCTCGGGCTCCTATCATTG |
| | | CCGTGACTCGAAATCCCCAGACTGCTCGCCAGGCCCATCTGTACCGTGGCATCTTCCCT |
| | | GTGCTGTGTAAGGATGCCGTGCTGAATGCCTGGGCTGAGGATGTCGACCTTCGTGTAAA |
| | | CTTGGCCATGGATGTTGGCAAGGCCCGAGGCTTCTTCAAGAAGGGAGATGTGGTCATTG |
| | | TGCTGACCGGGTGGCGCCCTGGCTCTGGATTCACCAACACCATGCGTGTAGTGCCTGTA |
| | | CCTTGA |

An RNA molecule, or a modRNA molecule, according to known codon degeneracy, may have a sequence of nucleotides that may be translated into a given amino acid sequence. Any such RNA or modRNA molecule may be used in accordance with the present disclosure where an RNA or modRNA identified by a polypeptide it encodes is referred to. Examples of amino acid sequence for polypeptides encoded by RNA or modRNA molecules disclosed herein are shown in Table 2.

TABLE 2
Amino acid sequences

| | SEQ | |
|---|---|---|
| Polypeptide | ID | |
| Molecule | NO | amino acid sequence |
| Mouse | 37 | MYQSLAMAANHGPPPGAYEAGGPGAFMHSAGAASSPVYVPTPRVPSS |
| GATA4 | | VLGLSYLQGGGSAAAAGTTSGGSSGAGPSGAGPGTQQGSPGWSQAGA |
| | | EGAAYTPPPVSPRFSFPGTTGSLAAAAAAAAAAAREAAYGSGGGAGA |
| | | GLAGREQYGRPGFAGSYSSPYPAYMADVGASWAAAAAASAGPFDSPV |
| | | LHSLPGRANPGRHPNLVDMFDDFSEGRECVNCGAMSTPLWRRDGTGH |
| | | YLCNACGLYHKMNGINRPLIKPQRRLSASRRVGLSCANCQTTTTTLW |
| | | RRNAEGEPVCNACGLYMKLHGVPRPLAMRKEGIQTRKRKPKNLNKSK |
| | | TPAGPAGETLPPSSGASSGNSSNATSSSSSSEEMRPIKTEPGLSSHY |
| | | GHSSSMSQTFSTVSGHGPSIHPVLSALKLSPQGYASPVTQTSQASSK |
| | | QDSWNSLVLADSHGDIITA* |
| Mouse | 38 | MGRKKIQITRIMDERNRQVTFTKRKFGLMKKAYELSVLCDCEIALII |
| Mef2c | | FNSTNKLFQYASTDMDKVLLKYTEYNEPHESRTNSDIVETLRKKGLN |
| | | GCDSPDPDADDSVGHSPESEDKYRKINEDIDLMISRQRLCAVPPPSF |
| | | EMPVTIPVSSHNSLVYSNPVSTLGNPNLLPLAHPSLQRNSMSPGVTH |
| | | RPPSAGNTGGLMGGDLTSGAGTSAGNGYGNPRNSPGLLVSPGNLNKN |
| | | IQAKSPPPMNLGMNNRKPDLRVLIPPGSKNTMPSVNQRINNSQSAQS |
| | | LATPVVSVATPTLPGQGMGGYPSAISTTYGTEYSLSSADLSSLSGFN |
| | | TASALHLGSVTGWQQQHLHNMPPSALSQLGACTSTHLSQSSNLSLPS |
| | | TQSLSIKSEPVSPPRDRTTTPSRYPQHTTRHEAGRSPVDSLSSCSSS |
| | | YDGSDREDHRNEFHSPIGLTRPSPDERESPSVKRMRLSEGWAT* |
| Mouse | 39 | MADTDEGFGLARTPLEPDSKDRSCDSKPESALGAPSKSPSSPQAAFT |
| Tbx5 | | QQGMEGIKVFLHERELWLKFHEVGTEMIITKAGRRMFPSYKVKVTGL |
| | | NPKTKYILLMDIVPADDHRYKFADNKWSVTGKAEPAMPGRLYVHPDS |
| | | PATGAHWMRQLVSFQKLKLTNNHLDPFGHIILNSMHKYQPRLHIVKA |
| | | DENNGFGSKNTAFCTHVFPETAFIAVTSYQNHKITQLKIENNPFAKG |
| | | FRGSDDLELHRMSRMQSKEYPVVPRSTVRHKVTSNHSPFSSETRALS |
| | | TSSNLGSQYQCENGVSGPSQDLLPPPNPYPLAQEHSQIYHCTKRKDE |
| | | ECSSTEHPYKKPYMEISPSEEDTFYRSGYPQQQGLSTSYRTESAQRQ |
| | | ACMYASSAPPSEPVPSLEDISCNTWPSMPSYSSCTVTTVQPMDRLPY |
| | | QHFSAHFTSGPLVPRLAGMANHGSPQLGEGMFQHQTSVAHQPVVRQC |
| | | GPQTGLQSPGGLQPPEFLYTHGVPRTLSPHQYHSVHGVGMVPEWSEN |
| | | S* |
| Mouse | 40 | MSLVGGFPHHPVVHHEGYPFAAAAAAAAAAAASRCSHEENPYFHGWL |
| Hand2 | | IGHPEMSPPDYSMALSYSPEYASGAAGLDHSHYGGVPPGAGPPGLGG |
| | | PRPVKRRGTANRKERRRTQSINSAFAELRECIPNVPADTKLSKIKTL |
| | | RLATSYIAYLMDLLAKDDQNGEAEAFKAEIKKTDVKEEKRKKELNEI |
| | | LKSTVSSNDKKTKGRTGWPQHVWALELKQ* |
| ASAH1 | 41 | MPGRSCVALVLLAAAVSCAVAQHAPPWTEDCRKSTYPPSGPTYRGAV |
| | | PWYTINLDLPPYKRWHELMLDKAPVLKVIVNSLKNMINTFVPSGKIM |
| | | QVVDEKLPGLLGNFPGPFEEEMKGIAAVTDIPLGEIISFNIFYELFT |
| | | ICTSIVAEDKKGHLIHGRNMDFGVFLGWNINNDTWVITEQLKPLTVN |
| | | LDFQRNNKTVFKASSFAGYVGMLTGFKPGLFSLTLNERFSINGGYLG |
| | | ILEWILGKKDVMWIGFLTRTVLENSTSYEEAKNLLTKTKILAPAYFI |
| | | LGGNQSGEGCVITRDRKESLDVYELDAKQGRWYVVQTNYDRWKHPFF |
| | | LDDRRTPAKMCLNRTSQENISFETMYDVLSTKPVLNKLTVYTTLIDV |
| | | TKGQFETYLRDCPDPCIGW* |
| dnTGFB | 42 | MGRGLLRGLWPLHIVLWTRIASTIPPHVQKSVNNDMIVTDNNGAVKF |
| | | PQLCKFCDVRFSTCDNQKSCMSNCSITSICEKPQEVCVAVWRKNDEN |
| | | ITLETVCHDPKLPYHDFILEDAASPKCIMKEKKKPGETFFMCSCSSD |
| | | ECNDNIIFSEEYNTSNPDLLLVIFQVTGISLLPPLGVAISVIIIFYC |
| | | YRVGSMDYKDDDDK* |
| dnWnt8a | 43 | MQNTTLFILATLLIFCPFFTASAWSVNNFLMTGPKAYLTYSASVAVG |
| | | AQNGIEECKYQFAWERWNCPESTLQLATHNGLRSATRETSFVHAISS |
| | | AGVMYTLTRNCSMGDFDNCGCDDSRNGRIGGRGWVWGGCSDNAEFGE |
| | | RISKLFVDGLETGQDARALMNLHNNEAGRLAVKETMKRTCKCHGISG |
| | | SCSIQTCWLQLAEFRDIGNPLKIKHDQALKLEMDXXK* |
| Human | 44 | MYQSLAMAANHGPPPGAYEAGGPGAFMHGAGAASSPVYVPTPRVPSS |
| GATA4 | | VLGLSYLQGGGAGSASGGASGGSSGGAASGAGPGTQQGSPGWSQAGA |
| | | DGAAYTPPPVSPRFSFPGTTGSLAAAAAAAAAAAREAAYSSGGGAGA |
| | | GLAGREQYGRAGFAGSYSSPYPAYMADVGASWAAAAAASAGPFDSPV |
| | | LHSLPGRANPAARHPNLVDMFDDFSEGRECVNCGAMSTPLWRRDGTG |
| | | HYLCNACGLYHKMNGINRPLIKPQRRLSASRRVGLSCANCQTTTTTL |
| | | WRRNAEGEPVCNACGLYMKLHGVPRPLAMRKEGIQTRKRKPKNLNKS |
| | | KTPAAPSGSESLPPASGASSNSSNATTSSSEEMRPIKTEPGLSSHYG |
| | | HSSSVSQTFSVSAMSGHGPSIHPVLSALKLSPQGYASPVSQSPQTSS |
| | | KQDSWNSLVLADSHGDIITA* |
| Human | 45 | MGRKKIQITRIMDERNRQVTFTKRKFGLMKKAYELSVLCDCEIALII |
| Mef2c | | FNSTNKLFQYASTDMDKVLLKYTEYNEPHESRTNSDIVEALNKKENK |
| | | GCESPDPDSSYALTPRTEEKYKKINEEFDNMIKSHKIPAVPPPNFEM |
| | | PVSIPVSSHNSLVYSNPVSSLGNPNLLPLAHPSLQRNSMSPGVTHRP |
| | | PSAGNTGGLMGGDLTSGAGTSAGNGYGNPRNSPGLLVSPGNLNKNMQ |
| | | AKSPPPMNLGMNNRKPDLRVLIPPGSKNTMPSVNQRINNSQSAQSLA |
| | | TPVVSVATPTLPGQGMGGYPSAISTTYGTEYSLSSADLSSLSGFNTA |
| | | SALHLGSVTGWQQQHLHNMPPSALSQLGACTSTHLSQSSNLSLPSTQ |
| | | SLNIKSEPVSPPRDRTTTPSRYPQHTRHEAGRSPVDSLSSCSSSYDG |
| | | SDREDHRNEFHSPIGLTRPSPDERESPSVKRMRLSEGWAT* |
| Human | 46 | MADADEGFGLAHTPLEPDAKDLPCDSKPESALGAPSKSPSSPQAAFT |
| Tbx5 | | QQGMEGIKVFLHERELWLKFHEVGTEMIITKAGRRMFPSYKVKVTGL |
| | | NPKTKYILLMDIVPADDHRYKFADNKWSVTGKAEPAMPGRLYVHPDS |
| | | PATGAHWMRQLVSFQKLKLTNNHLDPFGHIILNSMHKYQPRLHIVKA |
| | | DENNGFGSKNTAFCTHVFPETAFIAVTSYQNHKITQLKIENNPFAKG |
| | | FRGSDDMELHRMSRMQSKEYPVVPRSTVRQKVASNHSPFSSESRALS |
| | | TSSNLGSQYQCENGVSGPSQDLLPPPNPYPLPQEHSQIYHCTKRKEE |
| | | ECSTTDHPYKKPYMETSPSEEDSFYRSSYPQQQGLGASYRTESAQRQ |
| | | ACMYASSAPPSEPVPSLEDISCNTWPSMPSYSSCTVTTVQPMDRLPY |
| | | QHFSAHFTSGPLVPRLAGMANHGSPQLGEGMFQHQTSVAHQPVVRQC |
| | | GPQTGLQSPGTLQPPEFLYSHGVPRTLSPHQYHSVHGVGMVPEWSDN |
| | | S* |
| Human | 47 | MSLVGGFPHHPVVHHEGYPFAAAAAAAAAAAASRCSHEENPYFHGWL |
| Hand2 | | IGHPEMSPPDYSMALSYSPEYASGAAGLDHSHYGGVPPGAGPPGLGG |
| | | PRPVKRRGTANRKERRRTQSINSAFAELRECIPNVPADTKLSKIKTL |
| | | RLATSYIAYLMDLLAKDDQNGEAEAFKAEIKKTDVKEEKRKKELNEI |
| | | LKSTVSSNDKKTKGRTGWPQHVWALELKQ* |
| Human | 48 | MTLLGSEHSLLIRSKFRSVLQLRLQQRRTQEQLANQGIIPPLKRPAE |
| Myocardin | | FHEQRKHLDSDKAKNSLKRKARNRCNSADLVNMHILQASTAERSIPT |
| | | AQMKLKRARLADDLNEKIALRPGPLELVEKNILPVDSAVKEAIKGNQ |
| | | VSFSKSTDAFAFEEDSSSDGLSPDQTRSEDPQNSAGSPPDAKASDTP |
| | | STGSLGTNQDLASGSENDRNDSASQPSHQSDAGKQGLGPPSTPIAVH |
| | | AAVKSKSLGDSKNRHKKPKDPKPKVKKLKYHQYIPPDQKAEKSPPPM |
| | | DSAYARLLQQQQLFLQLQILSQQQQQQQHRFSYLGMHQAQLKEPNEQ |
| | | MVRNPNSSSTPLSNTPLSPVKNSFSGQTGVSSFKPGPLPPNLDDLKV |
| | | SELRQQLRIRGLPVSGTKTALMDRLRPFQDCSGNPVPNFGDITTVTF |
| | | PVTPNTLPNYQSSSSTSALSNGFYHFGSTSSSPPISPASSDLSVAGS |
| | | LPDTFNDASPSFGLHPSPVHVCTEESLMSSLNGGSVPSELDGLDSEK |
| | | DKMLVEKQKVINELTWKLQQEQRQVEELRMQLQKQKRNNCSEKKPLP |
| | | FLAASIKQEEAVSSCPFASQVPVKRQSSSSECHPPACEAAQLQPLGN |
| | | AHCVESSDQTNVLSSTFLSPQCSPQHSPLGAVKSPQHISLPPSPNNP |
| | | HFLPSSSGAQGEGHRVSSPISSQVCTAQNSGAHDGHPPSFSPHSSSL |
| | | HPPFSGAQADSSHGAGGNPCPKSPCVQQKMAGLHSSDKVGPKFSIPS |
| | | PTFSKSSSAISEVTQPPSYEDAVKQQMTRSQQMDELLDVLIESGEMP |
| | | ADAREDHSCLQKVPKIPRSSRSPTAVLTKPSASFEQASSGSQIPFDP |
| | | YATDSDEHLEVLLNSQSPLGKMSDVTLLKIGSEEPHFDGIMDGFSGK |
| | | AAEDLFNAHEILPGPLSPMQTQFSPSSVDSNGLQLSFTESPWETMEW |
| | | LDLTPPNSTPGFSALTTSSPSIFNIDFLDVTDLNLNSSMDLHLQQW* |

Another aspect relates to a pharmaceutical composition including any of the foregoing compositions or examples thereof and a pharmaceutically acceptable carrier.

This aspect is carried out in accordance with the previously described aspects.

A composition as described herein may be applied to a subject, such as a mammal, and a number of cardiomyocytes present in the subject's heart is increased, after a cardiac injury or insult, compared to a subject subjected to such injury or insult but without being treated with the composition. An injury or insult may include myocardial infarction, reperfusion injury, ischemic damage, or stroke. A subject experiencing such injury or insult may be in need of a treatment to increase a number of cardiomyocytes, such as to prevent, reduce, minimize, or otherwise counteract deleterious effects on cardiac health, structure, or function. Applying a composition to a subject may also promote wound healing, a treatment which such subject may be in need of following cardiac injury or insult.

A composition may be administered as a formulation in combination with one or more pharmaceutically acceptable carrier, excipient, or additive. A carrier, excipient, or additive may be “acceptable” in the sense of being compatible with other ingredients of the formulation and not deleterious to the recipient thereof. Formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy.

In one embodiment, the composition further comprises a pharmaceutically acceptable carrier. “Pharmaceutically acceptable carriers” as used herein refer to conventional pharmaceutically acceptable carriers. See Remington's Pharmaceutical Sciences, by E. W. Martin, Mack Publishing Co., Easton, Pa., 15th Edition (1975), which is hereby incorporated by reference in its entirety (describing compositions suitable for pharmaceutical delivery of the inventive compositions described herein). In particular, a pharmaceutically acceptable carrier as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits. In one embodiment, the pharmaceutically acceptable carrier is selected from the group consisting of a liquid filler, a solid filler, a diluent, an excipient, a solvent, and an encapsulating material.

Pharmaceutically acceptable carriers (e.g., additives such as diluents, immunostimulants, adjuvants, antioxidants, preservatives and solubilizing agents) are nontoxic to the cell or subject being exposed thereto at the dosages and concentrations employed. Examples of pharmaceutically acceptable carriers include water, e.g., buffered with phosphate, citrate and another organic acid. Representative examples of pharmaceutically acceptable excipients that may be useful in the present disclosure include antioxidants such as ascorbic acid; low molecular weight (less than about 10 residues) polypeptides; proteins, such as serum albumin, gelatin, or immunoglobulins; adjuvants (selected so as to avoid adjuvant-induced toxicity, such as a (3-glucan as described in U.S. Pat. No. 6,355,625, which is hereby incorporated by reference in its entirety, or a granulocyte colony stimulating factor (GCSF)); hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants such as TWEEN®, polyethylene glycol (PEG), and PLURONICS®.

In various embodiments, the compositions according to the disclosure may be formulated for delivery via any route of administration. The route of administration may refer to any administration pathway known in the art, including but not limited to intracardiac, aerosol, nasal, oral, transmucosal, transdermal, subcutaneous, or parenteral. Parenteral refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or in the form of lyophilized powders.

In one embodiment, the composition may further comprise an adjuvant. Suitable adjuvants are known in the art and include, without limitation, flagellin, Freund's complete or incomplete adjuvant, aluminum hydroxide, lysolecithin, pluronic polyols, polyanions, peptides, oil emulsion, dinitrophenol, iscomatrix, and liposome polycation DNA particles. In one embodiment, the composition is formulated for increasing a ratio of a number of cardiomyocytes to a number of non-cardiomyocytes, treatment of cardiac injury, stimulating vascular regeneration following ischemic damage, treating stroke, and/or enhancing wound healing.

A method in accordance with the present disclosure may include bringing into association a composition as disclosed (“active ingredient”) with a carrier which constitutes one or more accessory ingredients. In general, formulations may be prepared by uniformly and intimately bringing into association an active ingredient with liquid carriers or finely divided solid carriers or both and then, if necessary, shaping the product into the desired formulation. Formulations of the present disclosure suitable for oral administration may be presented as discrete units such as capsules, cachets or tablets each containing a predetermined amount of an active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

In some examples, the active compounds may be incorporated with excipients and used in the form of ingestible tablets, buccal tablets, troches, capsules, elixirs, suspensions, syrups, wafers, and the like. Tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

A tablet maybe made by compression or molding, optionally with one or more accessory ingredients. Compressed tablets may be prepared by compressing in a suitable machine an active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. Tablets may optionally be coated or scored and may be formulated so as to provide sustained, delayed or controlled release of an active ingredient therein.

Formulations for parenteral administration include aqueous and non-aqueous sterile injection solutions which may contain anti-oxidants, buffers, bacteriostats and solutes which render a formulation isotonic with the blood of an intended recipient. Formulations for parenteral administration also may include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

A formulation may include different types of carriers depending on whether it is to be administered in solid, liquid or aerosol form, and whether it needs to be sterile for such routes of administration as injection. A composition, as a formulation, may be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, which is hereby incorporated by reference in its entirety).

A carrier, excipient, or additive may include a composition to aid in cellular uptake, transportation or transfer of modRNA across a cell membrane to permit intracellular translation of a protein product therefrom. Various cell penetrating peptides, nanoparticles, lipoplexed configurations, and other carriers for enhanced cellular uptake may be used. Commercially available examples include RNA IMAX™, MESSENGER MAX™, JET MESSENGER™, and TRANS IT™.

A composition may be administered to a subject including an amount of a given modRNA, or relative amounts among multiple modRNA molecules encoding different peptides from each other applied in combination in a composition, in a therapeutically effective dose or amount. That is, amounts, or relative amounts, of different species to each other, within a composition may be sufficient to cause a beneficial effect on cardiac function, cardiac health, cardiac structure, wound healing, or measures of cardiac output indicative of or believed or known to correspond to positive health outcomes or positive cardiological health or function. An example may include an increase in cardiomyocytes in a heart of a subject, or improved measures of cardiac output such as increased ejection fraction (percent of the total amount of blood in the left ventricle is pushed out with each heartbeat), increased cardiac output (amount of blood the heart pumps from each ventricle per minute), increased stroke volume (volume of blood ejected from each ventricle due to the contraction of the heart), or fractional shortening (the degree of shortening of the left ventricular diameter between end-diastole and end-systole). In an example, multiple independent compositions may be administered, each including one or only a subset of types of modRNA to be administered, with the independent compositions administered in combination. In an example, such combination of compositions yields a relative ratio of various types of modRNA administered to cells or a subject as if all such subtypes were combined in a single composition.

Another aspect relates to a method for increasing a ratio of a number of cardiomyocytes to a number of non-cardiomyocytes within a population of cells including contacting said population of cells with the foregoing compositions or examples thereof. In one embodiment, the non-cardiomyocytes include cardiac fibroblasts.

This aspect is carried out in accordance with the previously described aspects.

Another aspect relates to a method for treating cardiac injury including administering to a patient in need of such treatment a therapeutically effective amount of the foregoing compositions or examples thereof. In one embodiment, the cardiac injury includes myocardial infarction. In another embodiment, the cardiac injury includes reperfusion injury.

This aspect is carried out in accordance with the previously described aspects.

Another aspect relates to a method for stimulating vascular regeneration following ischemic damage including contacting tissue damaged by ischemic damage with the foregoing compositions or examples thereof.

This aspect is carried out in accordance with the previously described aspects.

Another aspect relates to a method for treating of stroke including administering to a patient in need of such treatment a therapeutically effective amount of the foregoing compositions or examples thereof.

This aspect is carried out in accordance with the previously described aspects.

Another aspect relates to a method for enhancing wound healing comprising administering to a patient in need of such enhancement a therapeutically effective amount of the foregoing compositions or examples thereof.

This aspect is carried out in accordance with the previously described aspects.

Another aspect relates to a method for stimulating skeletal muscle regeneration comprising administering to a patient in need of said stimulation a therapeutically effective amount of the foregoing compositions or examples thereof.

As used herein, the term “reference level” refers to an amount of a substance, e.g., particular cell type (for example, stem cells), which may be of interest for comparative purposes. In some embodiments, a reference level may be the level or concentration of a population of a cell type expressed as an average of the level or concentration from samples of a control population of healthy (disease-free and/or pathogen-free) subjects. In other embodiments, the reference level may be the level in the same subject at a different time, e.g., before the present invention is employed, such as the level determined prior to the subject developing a disease, disease condition, and/or pathogenic infection, prior to initiating therapy, such as, for example, stem cell therapy, or earlier in the therapy. Mammalian subjects according to this aspect of the present invention include, for example, human subjects, equine subjects, porcine subjects, feline subjects, and canine subjects. Human subjects are particularly preferred.

For purposes of this and other aspects of the disclosure, the target “subject” encompasses any vertebrate, such as an animal, preferably a mammal, more preferably a human. In the context of administering a composition of the disclosure for purposes of increasing the ratio of a number of cardiomyocytes to a number of noncardiomyocytes, treating cardiac injury, stimulating vascular regeneration, treating stroke, and/or enhancing wound healing, a target subject encompasses any subject that has or is at risk of having ischemic heart disease, a lower ratio of cardiomyocytes to noncardiomyocytes (as compared to a reference level), cardiac injury, vascular degeneration, stroke, and/or wound(s) caused by any of the conditions described herein. Particularly susceptible subjects include adults and elderly adults. However, any infant, juvenile, adult, or elderly adult that has or is at risk of having any of the conditions described herein can be treated in accordance with the methods of the present disclosure. In one embodiment, the subject is an infant, a juvenile, or an adult.

As used herein, the phrase “therapeutically effective amount” means an amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment. The term “treatment” or “treat” may include effective inhibition, suppression or cessation of ischemic heart disease, a lower ratio of cardiomyocytes to noncardiomyocytes (as compared to a reference level), cardiac injury, vascular degeneration, stroke, and/or wound(s) caused by any of the conditions described herein, so as to prevent or delay the onset, retard the progression, or ameliorate the symptoms of the ischemic heart disease, a lower ratio of cardiomyocytes to noncardiomyocytes (as compared to a reference level), cardiac injury, vascular degeneration, stroke, and/or wound(s) caused by any of the conditions described herein.

As used herein a sample may include any sample obtained from a living system or subject, including, for example, blood, serum, and/or tissue. In one embodiment, a sample is obtained through sampling by minimally invasive or non-invasive approaches (for example, by urine collection, stool collection, blood drawing, needle aspiration, and other procedures involving minimal risk, discomfort, or effort). Alternatively, samples may be gaseous (for example, breath that has been exhaled) or liquid fluid. Liquid samples may include, for example, urine, blood, serum, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, intestinal secretions, nasal excretions, and other liquids. Samples may also include a clinical sample such as serum, plasma, other biological fluid, or tissue samples, and also includes cells in culture, cell supernatants and cell lysates. In one embodiment, the sample is selected from the group consisting of whole blood, serum, urine, and nasal excretion. Samples may be in vivo or ex vivo.

In one embodiment, the method includes administering one or more additional agents which treat ischemic heart disease, a lower ratio of cardiomyocytes to noncardiomyocytes (as compared to a reference level), cardiac injury, vascular degeneration, stroke, and/or wound(s) caused by any of the conditions described herein in the subject.

As used herein, the term “simultaneous” therapeutic use refers to the administration of at least one additional agent beyond the compositions described herein, optionally, by the same route and at the same time or at substantially the same time. As used herein, the term “separate” therapeutic use refers to an administration of at least one additional agent beyond the compositions described herein at the same time or at substantially the same time by different routes. As used herein, the term “sequential” therapeutic use refers to administration of at least one additional agent beyond the compositions described herein at different times, the administration route being identical or different. More particularly, sequential use refers to the whole administration of the additional agent before administration of the compositions described herein. It is thus possible to administer the additional agent over several minutes, hours, or days before applying the compositions described herein.

In one embodiment, the additional agent may include, for example, one or more antibiotic compound; one or more antimicrobial compound; one or more antibody; one or more biocidal agent; one or more nanoparticle; one or more self-assembling nanoparticle; one or more viral particle; one or more bacteriophage particle; one or more bacteriophage DNA; genetic material including but not limited to a plasmid, RNA, mRNA, siRNA, and an aptamer; one or more chemotherapy agent; one or more growth factor; one or more synthetic scaffold including but not limited to hydrogel and others; one or more natural scaffold including but not limited to collagen gel and decellularized tissue (whole, dissolved, denatured, or powdered); one or more electrode, one or more drug or pharmaceutical compound including but not limited to an anti-inflammatory agent, an inflammatory agent, a pain blocking agent, and a numbing agent; one or more microbes, and one or more bacteria.

In the following description, reference is made to the accompanying drawings that form a part hereof, and in which is shown by way of illustration specific embodiments which may be practiced. These embodiments are described in detail to enable those skilled in the art to practice the disclosure, and it is to be understood that other embodiments may be utilized and that structural, logical and electrical changes may be made without departing from the scope of the present disclosure. The following description of example embodiments is, therefore, not to be taken in a limited sense.

The present disclosure may be further illustrated by reference to the following examples.

### EXAMPLES

The following examples are intended to illustrate, but by no means are intended to limit, the scope of the present disclosure as set forth in the appended claims.

### Example 1—Materials and Methods

Mice—All animal procedures were performed under protocols approved by the Icahn School of Medicine at Mount Sinai Institutional Animal Care and Use Committee (IACUC). For in vitro experiments, cardiac fibroblasts were isolated from P0-P4 α-myosin heavy chain-mCherry (α-MHC-mCherry) transgenic neonate hearts (mice purchased from Jackson Laboratories). For lineage-tracing studies, Tnnt2MerCreMer/+/R26mTmG/+mice were generated by crossing TnnT-Cre mice (gifted by Dr. Chen-Leng Cai) and Rosa26mTmG (Jackson Laboratory) mice. At 11 weeks of age, tamoxifen dissolved in sesame oil was injected 3 for consecutive days at 0.1 mg tamoxifen per 1 g body weight. Myocardial infarction (MI) was induced by permanently ligating the left anterior descending (LAD) artery a week after tamoxifen injections. Male and female mice were randomized into four different groups (Luc, 7G, 7G GMT(Hx2) and 7G G(Mx2)TH) and modRNA was injected directly into the myocardium during open chest surgery. For Echo, MRI and long-term survival analyses, 8-10-week-old CFW mice were treated with four modRNA types post induction of MI and allowed to recover for 6 months in the animal facility. Deaths were monitored and documented. For limb ischemic studies, 4 months old ApoE−/− mice (males and females) were used. A unilateral hindlimb ischemia was induced via the ligation of the left femoral artery by isolating the femoral artery from the femoral nerve and vein, and then cut at the level of the internal iliac artery and popliteal artery. Following to artery ligation, randomized mice received the injection of Luc and 7G at 3 different sites in the gastrocnemius muscle.

modRNA synthesis—All modRNA was generated by in vitro transcription of plasmid templates (GeneArt, Thermo Fisher Scientific). The full list of open reading frame sequences used to make modRNA for this study can be found in Table 1 shown herein. The transcription step involved a customized ribonucleotide blend of anti-reverse cap analog; 30-O-Me-m7G(50) ppp(50)G (6 mM, TriLink Biotechnologies); guanosine triphosphate (1.5 mM, Life Technologies); adenosine triphosphate (7.5 mM, Life Technologies); cytidine triphosphate (7.5 mM, Life Technologies) and N1-methylpseudouridine-5-triphosphate (7.5 mM, TriLink Biotechnologies). Next, modRNA was purified with the Megaclear kit (Life Technologies) and treated with Antarctic Phosphatase (New England Biolabs). To eliminate any remaining impurities, modRNA was re-purified with the Megaclear kit and quantified using a Nanodrop spectrometer (Thermo Scientific). Lastly, modRNA was precipitated with ethanol and ammonium acetate and resuspended in 10 mM Tris-HCl and 1 mM EDTA.

modRNA transfection—For in vitro transfection, cells plated in 24-well plates and 6-well plates were transfected with 2.5 ug of mRNA and bug of mRNA, respectively, encoding various genes using RNAiMAX transfection reagent (Life Technologies) in accordance with the instructions provided by the RNAiMAX manufacturer. In vivo gene delivery was performed according to previously published methods. Simeonov et al., “Direct Reprogramming of Human Fibroblasts to Hepatocyte-Like Cells by Synthetic Modified mRNAs,” PLoS One. 9:e100134 (2014), which is hereby incorporated by reference in its entirety. ModRNA was delivered using sucrose citrate buffer containing 20 μl of sucrose in nuclease-free water (0.3 g/ml), with 20 μl of citrate (0.1M pH=7; Sigma) mixed with 20 μl of different modRNA concentrations in saline to a total volume of 60 μl. The transfection mixture was directly injected into heart muscle surrounding the MI (two on either side of the ligation and one in the apex), with 20 μl at each site.

Cell culture: Mouse cardiac fibroblast culture—Hearts isolated from neonate mice were chopped into small pieces of approximately 1 mm³ size and digested for 20 mins on a rocker with collagenase type II in PBS and 0.25% (wt/vol) trypsin. Post digestion, the clumpy heart tissue was centrifuged at 600 g for 2 mins and plated in a 10 cm dish (3-5 hearts per dish) in fibroblast explant media (Iscove's modified Dulbecco medium with 20% FBS (IMDM)) at 37° C. After 30 mins of incubation, the plate was washed with PBS and cells were quenched with fresh media. When confluent, attached cells were washed with PBS, collected with 5 minute 0.05% trypsin treatment and quenched with fibroblast explant media. Cells were then filtered through a 70-μm filter and pellet was collected. Thereafter, the cells were sorted by fluorescence-activated cell sorting (FACS) for mCherry-negative cells, plated onto 6-well gelatin-coated plates at a concentration of 10000 cells/cm² and used fresh for all reprogramming studies. After 6 hours of modRNA transfection with RNAimax, media was replaced with cardiomyocyte induction media (iCM) comprising DMEM:M199 (4:1), 10% FBS, 1× non-essential amino acids (NEAA) and 1× penicillin/streptomycin. Thereafter, SMI SB431542 (2.6 μM) and XAV939 (5 μM) were added 24 h and 48 h post infection, respectively.

Cell culture: Human cell line—Normal human cardiac fibroblasts-ventricular (NHCF-V) were purchased from Lonza (CC-2904) and grown in Cardiac Fibroblast Growth Medium (Lonza, CC-4526). Once semi-confluent, cells were transfected with SV40-Large T modRNA 3 days before transfection with reprogramming genes to obtain a stable immortalized cell line. Cells were transfected with different modRNA and SMI (wherever mentioned) for 6 consecutive days for WB and 14 consecutive days for cardiomyocyte reprogramming assay. For WB experiments, cells were collected every day during Days 1-6, while reprogramming analyses (qPCR and ICC) were performed at day 14.

Immunofluorescence˜28 days post MI, mice hearts were harvested, and excess blood was removed by injecting 1 ml PBS in the right ventricular chamber. Hearts were fixed via overnight incubation in 4% PFA, with subsequent PBS washings for at least an hour. Hearts were then placed in 30% sucrose solution at 4° C. overnight. The following day, hearts were fixed in OCT and frozen at −80° C. Transversal 10 um-thick sections were made by cryostat and rehydrated in PBS for 5 min for immunostaining. All staining was performed on 3-8 hearts/group, with 2-3 sections/heart. For immunostaining mCherry-negative neonatal mouse non-CMs following modRNA treatment, cells were fixed on coverslips with 4% PFA for 15 min at room temperature, then washed 3 times with PBST. Cells/tissues were permeabilization with PBS with 0.1% triton ×100 (PBST) for 7 min followed by overnight staining with primary antibodies. The recommended concentrations of sacromeric α-actinin (Abcam, #9465), cardiac troponin I (Abcam, #7003) and CD31 (R&D Systems, #3628) diluted in PBST, GFP (Abcam, #13970) and tdTomato (Origene, #8181-200) were used. The next day, slides were washed with PBST (5 times for 4 min each) followed by incubation with a secondary antibody (Invitrogen, 1:200) diluted in PBST for 2 hours at room temperature. To remove the secondary antibody, the samples were further washed with PBST (3 times for 5 min each) and stained with Hoechst 33342 (1 μg/ml) diluted in PBST for 7 min. After 5 washes with PBST for 4 min each, slides were mounted with mounting medium (Vectashield) for imaging. Stained slides were stored at 4° C. The fluorescent images were taken on Zeiss fluorescent microscopy at 10×, 20× and 40× magnification.

Masson's trichome staining—Masson's trichome staining was performed to evaluate scar size in the LV post MI and modRNA treatments. The OCT frozen transverse heart sections were air dried for 30 min to 1 hr at room temperature before proceeding to staining. Slides were pre-stained with Bouin's Solution for 45 mins at 55 C. Next, slides were kept in Weigert's Iron Hematoxylin, Biebrich Scarlet-Acid Fucshin, Phosphotungstic/Phosphomolybdic Acid Solution and Aniline Blue Solution for the time suggested by manufacturer. Thereafter, tissue samples were differentiated with acetic acid for 2 mins and dehydrated through 95% ethyl alcohol and absolute ethyl alcohol. After being cleared using xylene, slides were mounted with Permount mounting media (Fisher Scientific). Images were collected using a bright field microscope and scar size analysis was done using ImageJ software.

H&E staining—H&E staining was performed according to standard protocol. The OCT frozen transverse heart sections were air dried for 30 min to 1 hr at room temperature, then hydrated in PBS for 10 mins. The slides were kept in Hematoxylin solution for 2 mins and washed with tap water for 5 mins. Thereafter, the sections were stained using eosin solution for 1 min and washed with tap water for 5 mins. The slides were transferred to PBS for 5 mins. Sections were then dehydrated in 100% ethanol and xylene for 1 min each. Finally, sections were mounted with Permount mounting media (Fisher Scientific). The images were taken on a bright field microscope.

Western blotting—Total protein from the respective cells or thawed tissues was isolated at given time points. 20 ug of protein from each sample was resolved using an SDS-PAG Electrophoresis system in 4%-15% Mini-PROTEAN TGX stain-free gels (Bio-Rad). The resulting bands were transferred onto polyvinylidene fluoride (PVDF) membranes (Bio-Rad). The membranes were blocked (5% BSA in Tris-buffered saline Tween 20 (TBS; 50 mM Tris-HCl [pH 7.4], 150 mM NaCl, 0.1% Tween 20) for 1 hr at room temperature and then incubated with primary antibodies diluted in 5% BSA in TBST overnight at 4 C. Anti-Flag (1:1,000, Sigma, #A8592); anti-Vegfa (1:1,000, Abcam, #51745); anti-GAPDH (horseradish peroxidase [HRP] conjugate 1:3,000, Cell Signaling, #8884) and mouse monoclonal anti-β-actin (horseradish peroxidase [HRP] conjugate 1:3,000, Cell Signaling, #12262) antibodies were used. Anti-rabbit and anti-mouse HRP-conjugated secondary antibodies were purchased from Cell Signaling. Antigen or antibody complexes were visualized with the ChemiDoc Touch imaging system (Bio-Rad).

RNA isolation and gene expression profiling using Real-time PCR—Quick RNA kit (Zymo Research) was used to isolate total RNA from the cells and ischemic mouse tissue at the aforementioned time points and reverse transcribed using iScript™ cDNA Synthesis Kit (Bio Rad) according to the manufacturer's instructions. Real-time qPCR analyses were performed on a Mastercycler Realplex 4 Sequence Detector (Eppendorf) using SYBR Green (PerfeCTa SYBR Green FastMix, QuantaBio). Data were normalized to GAPDH (in vitro experiments and in vivo limb tissue experiments), 18s (in vivo experiments for cardiac tissue), and B2M (for human experiments). Fold-changes in gene expression were determined by the ∂∂CT method and presented relative to an internal control. PCR primer sequences are shown in Table 3.

TABLE 3
Primer Sequences for qPCR in this study

| Gene | | |
|---|---|---|
| name | Forward primer | Reverse primer |
| GAPDH | AGGTCGGTGTGAACGGATTTG | TGTAGACCATGTAGTTGAGGT |
| | (SEQ ID NO: 49) | CA (SEQ ID NO: 50) |
| 18S | TAACGAACGAGACTCTGGCAT | CGGACATCTAAGGGCATCACA |
| | (SEQ ID NO: 51) | G (SEQ ID NO: 52) |
| MYH6 | GGTGTGCGGATAAAAGACGG | TGGTCCACCTAAGAGGGTGT |
| | (SEQ ID NO: 53) | (SEQ ID NO: 54) |
| Tnnt2 | CTGAGACAGAGGAGGCCAAC | TTCCGCTCTGTCTTCTGGAT |
| | (SEQ ID NO: 55) | (SEQ ID NO: 56) |
| Vegfa | CTGTGCAGGCTGCTGTAACG | GTTCCCGAAACCCTGAGGAG |
| | (SEQ ID NO: 57) | (SEQ ID NO: 58) |
| Vegfb | CCCCTGTGTCCCAGTTTG (SEQ ID | CGTCTATCCATGGCACCAC |
| | NO: 59) | (SEQ ID NO: 60) |
| Vegfc | CAGACAAGTTCATTCAATTATTAG | CATGTCTTGTTAGCTGCCTGA |
| | ACG (SEQ ID NO: 61) | (SEQ ID NO: 62) |
| Vegfd | GCAACTTTCTATGACACTGAAACA | TCTCTCTAGGGCTGCATTGG |
| | C (SEQ ID NO: 63) | (SEQ ID NO: 64) |
| Angiogenin | CACAGATGGCCTTGATGTTG (SEQ | CTCTGGCTCAGCATGACTCC |
| 1 | ID NO: 65) | (SEQ ID NO: 66) |
| Angiopoitin- | GGGGGAGGTTGGACAGTAA (SEQ | CATCAGCTCAATCCTCAGC |
| 1 | ID NO: 67) | (SEQ ID NO: 68) |
| Leptin | CAAGCAGTGCCTATCCAGA (SEQ | AAGCCCAGGAATGAAGTCCA |
| | ID NO: 69) | (SEQ ID NO: 70) |
| Proliferin | TTCAACCATGCTCCTGGATA (SEQ | GACCATTCCTCATTGCACAC |
| | ID NO: 71) | (SEQ ID NO: 72) |
| TgfB | GCTGCGCTTGCAGAGATTAAA | TTGCTGTACTGTGTGTCCAG |
| | (SEQ ID NO: 73) | (SEQ ID NO: 74) |
| HGF | GTAAATGGGATTCCAACACGAACA | TGTCGTGCAGTAAGAACCCAA |
| | A (SEQ ID NO: 75) | CTC (SEQ ID NO: 76) |
| Amphiregulin | GCCATTATGCAGCTGCTTTGGAGC | TGTTTTTCTTGGGCTTAATCAC |
| | (SEQ ID NO: 77) | CT(SEQ ID NO: 78) |
| FGF-a | GTTGACCCTACCATGTTCCCTTG | GCCAGCAGCATCTATGGGAC |
| | (SEQ ID NO: 79) | (SEQ ID NO: 80) |
| FGF-b | GAAACACTCTTCTGTAACACACTT | GTCAAACTACAACTCCAAGCAG |
| | (SEQ ID NO: 81) | (SEQ ID NO: 82) |
| EGF | GCAACTCCGTCCGGGCGAGGA | GAAGATGACTGTGGTCCCGGG |
| | (SEQ ID NO: 83) | (SEQ ID NO: 84) |
| TB4 | AAACCCGATATGGCTG AGATTG | GCCTGCTTGCTTCTCCTGTT |
| | (SEQ ID NO: 85) | (SEQ ID NO: 86) |
| PDGF | CCCCTGCCCATTCGGAGGAAGAG | TTGGCCACCTTGACGCTGCGG |
| | (SEQ ID NO: 87) | TG (SEQ ID NO: 88) |
| Human | TTGCCTTGCTGCTCTACCTCCA | GATGGCAGTAGCTGCGCTGAT |
| vegfa | (SEQ ID NO: 89) | A (SEQ ID NO: 90) |
| hB2M | TGCCTGCCGTGTGAACCATGT | TGCGGCATCTTCAAACCTCCAT |
| | (SEQ ID NO: 91) | GA (SEQ ID NO: 92) |

Echocardiography (Echo)—Transthoracic two-dimensional echocardiography was conducted 2 and 28 days after MI to assess LV dimensions and function using a GE Cares InSite (V7R5049) equipped with a 40 MHz mouse ultrasound probe. In a double-blind study (i.e. neither the surgeon nor the echography technician was aware of the treatment), Luc, 7G or 7G GMT (Hx2) were injected into CFW mice (8 to 12 weeks old). Mice were anesthetized with 1-2% isoflurane in air, and imaging was performed. The ejection fraction and fractional shortening were calculated as percentages from the diastolic volume (EDV) and end systolic volume (ESV) dimensions on an M-mode ultrasound scan. % fractional shortening was calculated as =(left ventricular internal dimension at end-diastole (LVIDd)—left ventricular internal dimension at end-systolic (LVIDs))/LVIDd*100. Echocardiograms were performed on 3-10 hearts/treatment group.

Magnetic Resonance Imaging (MRI)—In a double-blind study, CFW mice (8 weeks old) treated with Luc, 7G or 7G GMT (Hx2) modRNA were subjected to MRI assessment on day 28 post LAD ligation. Delayed-enhancement CINE images were obtained on a 7-T Bruker Pharmascan with cardiac and respiratory gating (SA Instruments). For imaging, mice were anesthetized with 1-2% isoflurane in air. To monitor optimal temperature during ECG, respiratory and temperature probes were placed on the mouse. Imaging was performed 10 to 20 min after IV injection of 0.3 mmol/kg gadolinium-diethylene triamine pentaacetic acid. A stack of 8 to 10 short-axis slices spanning from the apex to the base of the heart were acquired with an ECG-triggered and respiratory-gated FLASH sequence with the following parameters: echo time (TE) 2.7 msec with resolution of 200 μm×200 μm; slice thickness of 1 mm; 16 frames per R-R interval; 4 excitations with flip angle at 60°. After imaging, the obtained data were analyzed to calculate % ejection fraction, cardiac output, stroke volume and % MI size.

Immunodetection methods—Leaky vessel detection was performed on heart tissues isolated from mice 28 days post MI and modRNA injection. Isolectin B4 (0.5 mg/ml, Vector Lab) was used to stain endothelial cells in cryosections to determine capillary density. To evaluate blood vessel leakiness, a mixture of 250 ul of isolectin B4 and 250 ul 70 kD FITC-dextran beads (50 mg/ml, Sigma) was injected into the mouse tail vein. Hearts were collected 30 mins after injection and fixed overnight using 4% PFA. After 4 washes with PBS, hearts were placed in 30% sucrose overnight and frozen in OCT the following day. Sectioned heart tissue was evaluated for vessel leakiness under microscope.

Blood Flow analysis in Ischemic Limb Model—Using the Laser Doppler perfusion imaging (LDPI) analyzer (Moor Instruments, Axminster, UK), blood flow measurement was performed in the ligated and control limbs at predetermined time points. Hind limb hair were removed and the animals were kept on a heating pad at 37° C. to minimize temperature variation. Blood flow was recorded on day 0 (before ligation), 1, 7, 14 and 21 post-ischemic and modRNA administration. % blood perfusion was calculated by comparing the blood flow in the ischemic limbs to the control hind limbs.

Statistical analysis—Statistical significance was determined by Unpaired two-tailed t-test, One-way ANOVA, Tukey's Multiple Comparison Test, One-way ANOVA, Bonferroni post hoc test or Log-rank (Mantel-Cox) test for survival curves, as detailed in respective Figure legends. p-Value<0.05 was considered significant. All graphs represent average values, and values were reported as mean±standard error of the mean. Unpaired two-tailed t-test was based on assumed normal distributions. ****, P<0.0001 ***, P<0.001, **, P<0.01, *, P<0.05, N. S, Not Significant.

### Example 2—modRNA Compositions Reprogram Fibroblasts to Cardiomyocyte-Like Cells

Cardiac fibroblasts were isolated from mice bearing a transgene in which alpha-myosin heavy chain (alpha-MHC) promotor, a cardiomyocyte-enriched protein, drove expression of marker protein mCherry and transfected with different combinations of modRNA, including GATA4 plus Mef2c plus Tbx5 (GMT), GMT plus Hand2 (GMTH), GMT plus ASAH1 (GMTA), or GMT, GMTH, or GMTHA plus TGFB small molecule inhibitor SB431542 and Wnt8a small molecule inhibitor XAV939) (SMI). modRNAs included pseudouridine in place of uridine and were treated with a reagent to form 5′UTR Cap 1 structure (CLEANCAP™). All treatments increased the number of α-actinin-positive cells that also expressed mCherry, indicating adoption of a cardiomyocyte phenotype. The highest number of such cells was seen following treatment with GMTHA+SMI. Treatments also increased cardiac troponin (cTNT) expression, further signifying adoption of cardiomyocyte phenotype by the cardiac fibroblasts, with highest levels seen in combinations that included Hand2 modRNA. In other treatment conditions, inclusion of dnTGFB modRNA instead of SB431542 or dnWnt8a modRNA instead of XAV939 also increased mCherry expression and cTNT expression.

Highest levels of mCherry expression were seen following treatment with all seven of GATA4, Mef2c, Tbx5, Hand2, ASAH1, dnTGFB, and dnWnt8a modRNAs. Three weeks after transfections, cells receiving this treatment also showed spontaneous contractions, further confirming a cardiomyocyte-like phenotype. Cells received different relative amounts of different modRNA molecules, as set out in Table 4:

TABLE 4
Ratios of modRNA molecules in different treatments

| Treatment | | | | | | | |
|---|---|---|---|---|---|---|---|
| condition | GATA4 | Mef2c | Tbx5 | Hand2 | ASAH1 | dnTGFB | dnWnt8a |
| 7G | 1 | 1 | 1 | 1 | 1 | 1 | 1 |
| 7G (GMTH)x2 | 2 | 2 | 2 | 2 | 0.7 | 0.7 | 0.7 |
| 7G G(x2)MTH | 2 | 1 | 1 | 1 | 0.7 | 0.7 | 0.7 |
| 7G GM(x2)TH | 1 | 2 | 1 | 1 | 0.7 | 0.7 | 0.7 |
| 7G GMT(x2)H | 1 | 1 | 2 | 1 | 0.7 | 0.7 | 0.7 |
| 7G GMTH(x2) | 1 | 1 | 1 | 2 | 0.7 | 0.7 | 0.7 |
| 7G GM(x2)TH(x2) | 1 | 2 | 1 | 2 | 0.5 | 0.5 | 0.5 |

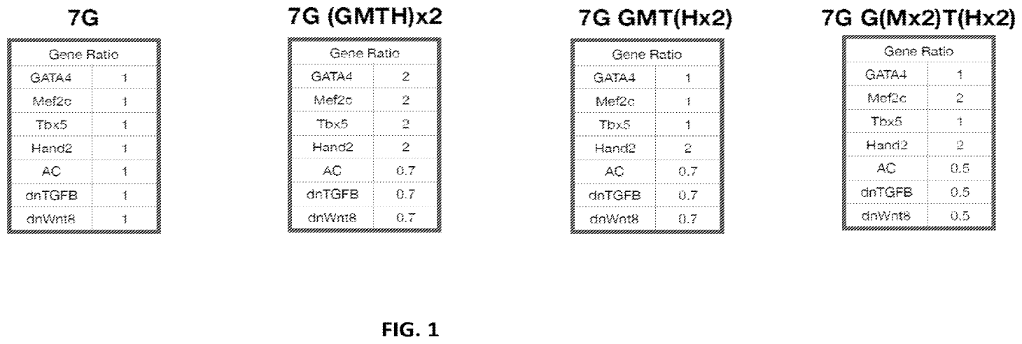

Some examples are illustrated in FIG. 1. All treatments caused an increase in cTNT and alpha-MHC expression. Interestingly, and surprisingly, increasing GATA4, Mef2c, Tbx5, or Hand2 by 50% (offset by 30% reductions in the remaining GATA4, Mef2c, Tbx5, or Hand2 modRNAs, depending on the example), eliminated the increases in cardiomyocyte protein expression. Transfection with additional modRNA molecules did not further increase.

### Example 3—modRNA Compositions in Mice Increase Cardiomyocyte-Like Cells and Improved Cardiac Health

Mice with the cardiac troponin T promoter driving expression of a tamoxifen-inducible Cre recombinase were crossed with transgenic mice carrying the mT/mG reporter driven by the Rosa26 promotor. Bi-transgenic mice treated with tamoxifen express cell membrane-localized tdTomato (mT) fluorescence except Cre recombinase expressing cells (and future cell lineages derived from these cells), in this case cardiomyocytes, which have cell membrane-localized EGFP (mG) fluorescence. Cardiomyocytes could therefore be identified by expression of cell membrane localized EGFP.

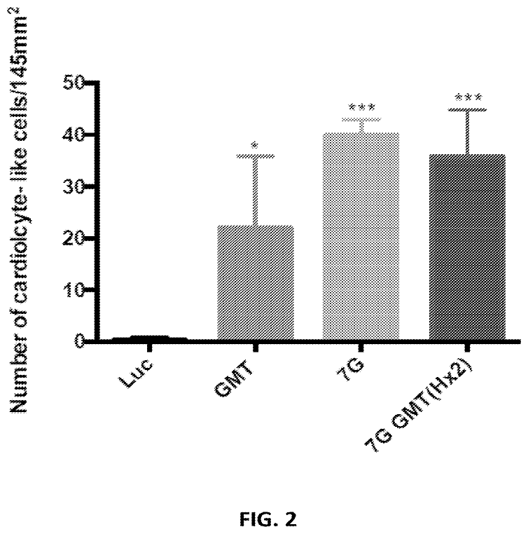

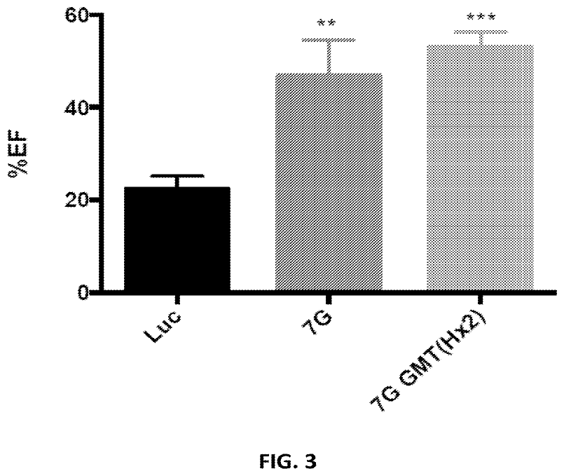

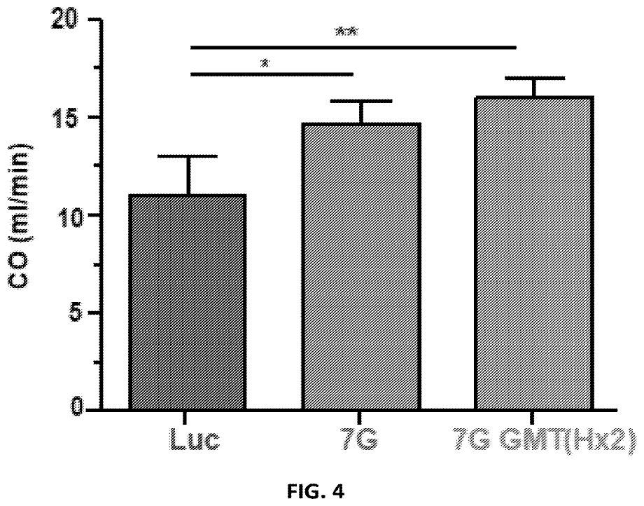

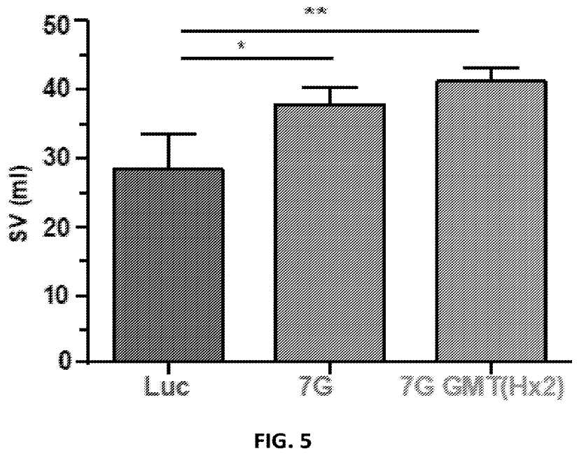

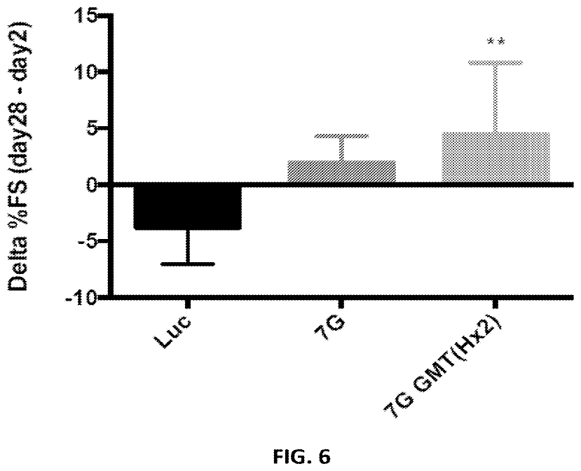

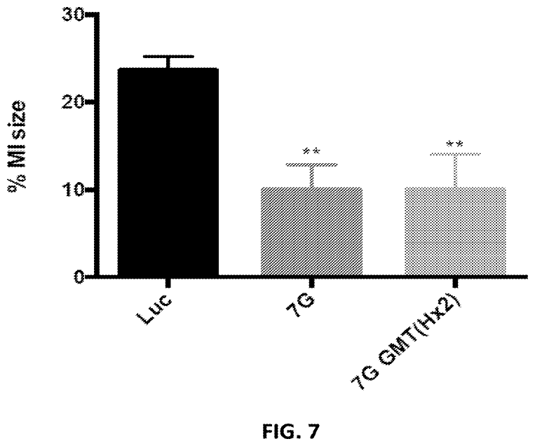

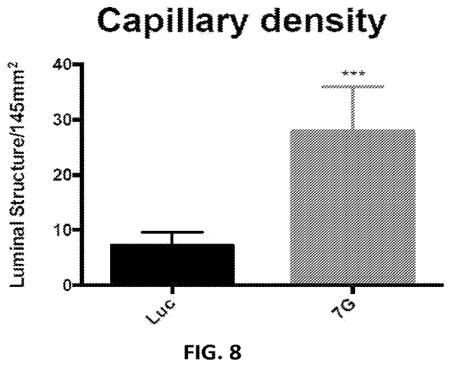

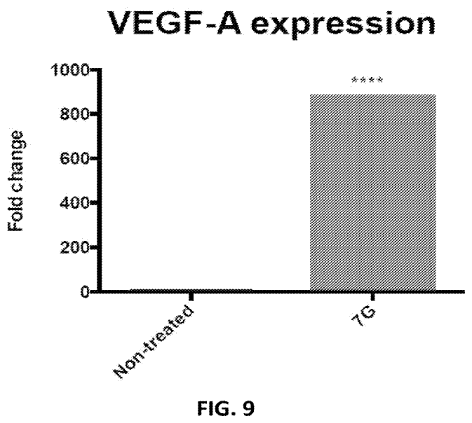

Mice were treated with tamoxifen at 8 weeks of age. At 10 weeks of age they were administered a myocardial infarction (MI) event (ligation of the left anterior descending artery). modRNA injections occurred at weeks 12 and 13 of age. At 15 weeks of age, heart function and cellular expression were tested. As shown in FIG. 2, treatment with 7G GMT(Hx2), 7G, or GMT alone all caused an increase in cardiomyocyte-like cells in an infarct are in mice with an MI event. As shown in FIG. 3, 7G and 7G GMT(Hx2) treatment significantly increased ejection fraction, indicating improved cardiac function compared to luciferase-transfected control mice. FIG. 4 shows that 7G GMT(Hx2) significantly increased cardiac output compared to controls, again indicating improved cardiac health. FIG. 5 shows that 7G GMT(Hx2) significantly increased stroke volume compared to controls, again indicating improved cardiac health. FIG. 6 shows that 7G GMT(Hx2) significantly increased fractional shortening compared to controls, again indicating improved cardiac health. FIG. 7 shows that 7G and 7G GMT(Hx2) cause significant reductions in infarct size. FIGS. 8 and 9 show increased capillary density and VEGF-A expression following 7G treatment.

### Example 4—Seven Gene modRNA Cocktail (7G) can Induce High-Efficiency Cardiac Reprogramming in Mouse or Human Non-CMs In Vitro

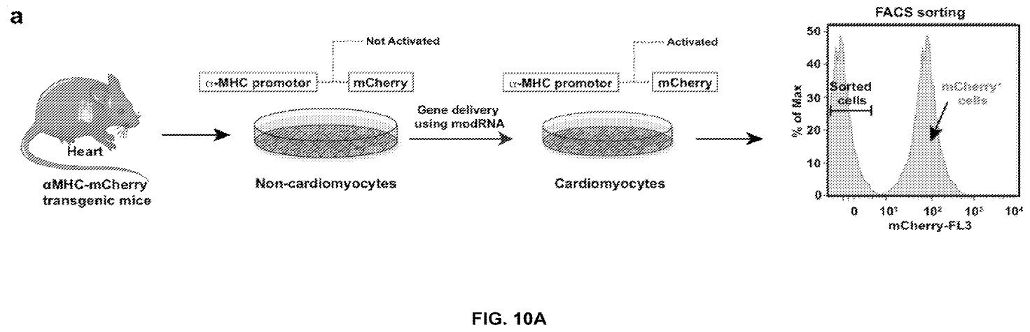

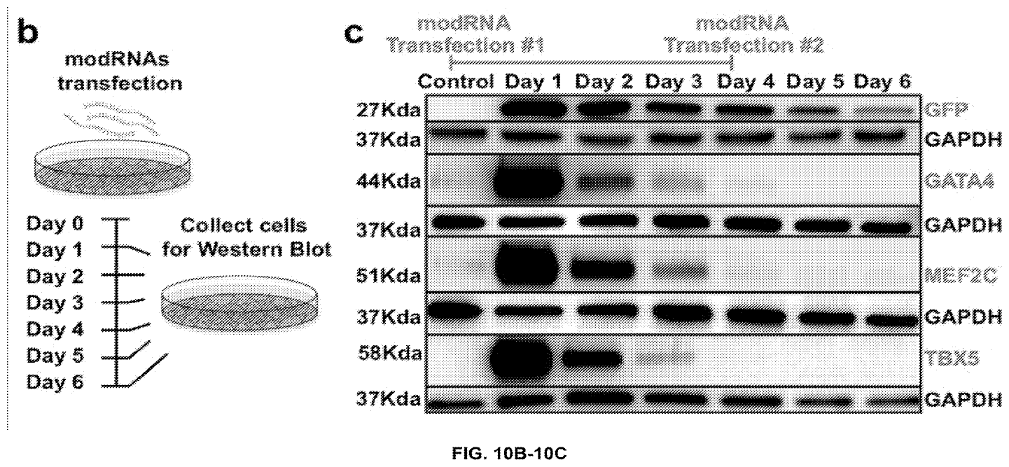

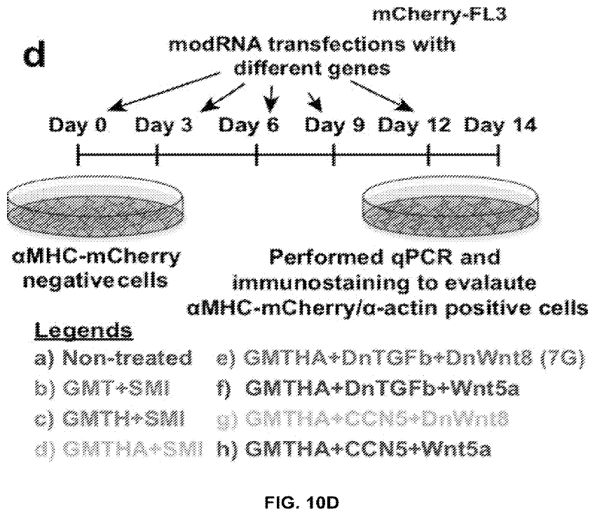

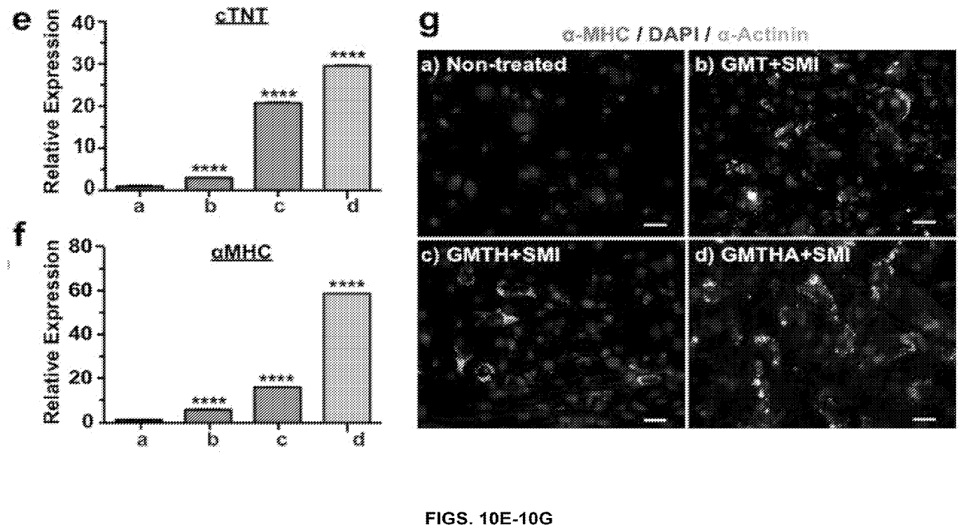

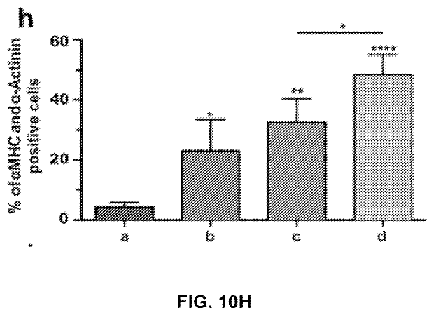

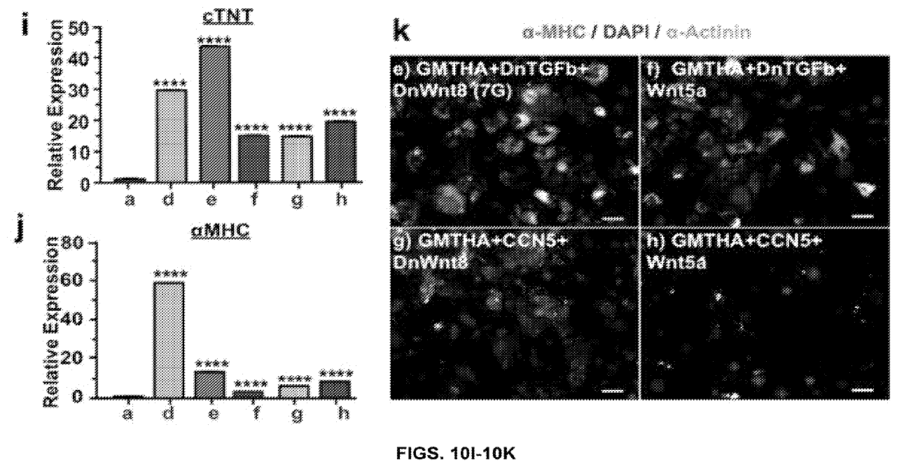

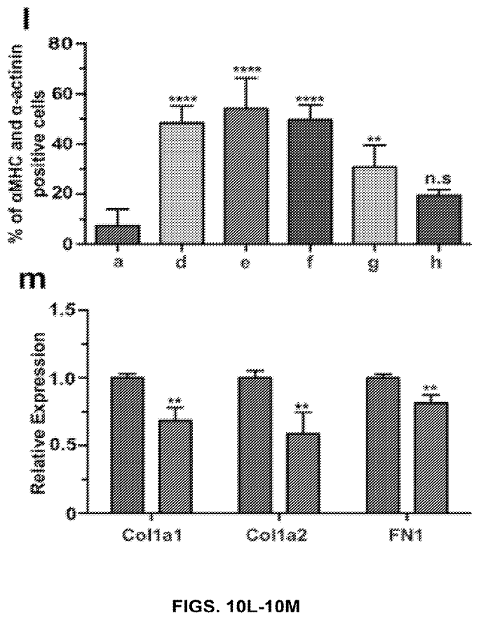

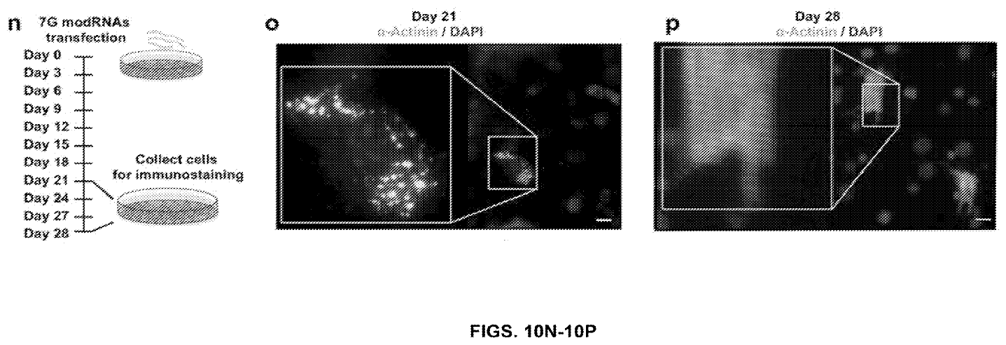

To demonstrate that modRNA technology can be used to reprogram adult mouse non-CMs into CM-like cells, a previously published in vitro lineage-tracing model was generated (Ieda et al., “Direct Reprogramming of Fibroblasts Into Functional Cardiomyocytes by Defined Factors,” Cell 142:375-86 (2010) and Qian et al., “In Vivo Reprogramming of Murine Cardiac Fibroblasts Into Induced Cardiomyocytes,” Nature 485:593-8 (2012), both of which are hereby incorporated by reference in their entirety) based on αMHC-mCherry transgenic mice. In this model, the CMs of mice carrying αMHC-mCherry express mCherry while non-CM are mCherry-negative. Using the enzymatic dissociation method, cardiac cells were isolated from neonate mouse hearts and cultured for 3 days before the mCherry-negative cells were sorted using FACS. Isolated mCherry-negative cells were plated and modRNA transfection began once the cells reached partial confluency. Successful cardiac reprogramming in vitro was confirmed by the expression of the mCherry reporter gene in the non-CM subset that upregulates αMHC, a CM-specific cell marker (FIG. 10A). As modRNA leads to transient gene expression, repeated transfections could replenish the gene expression in the transfected cells. To optimize the GMT transfection efficiency for reprogramming, non-CM cells were transfected with GFP or GMT modRNA (flag tagged) and measured their expression at different time points (day 1 through 6) using western blot (FIG. 10B). It was observed that GMT expression until day 3, but not 4 days post modRNA transfection (FIG. 10C). Thus, non-CMs that were isolated from αMHC-mCherry mice were transduced with GMT modRNA every 3 days for 14 days before evaluating cardiac reprogramming with qPCR and immunostaining (FIG. 10D).

GMT or GMTH driven cardiac reprogramming, could be enhanced by inhibiting TGFβ and/or WNT pathways. Abad et al., “Notch Inhibition Enhances Cardiac Reprogramming by Increasing MEF2C Transcriptional Activity,” Stem Cell Reports 8:548-560 (2017); Ifkovits et al., “Inhibition of TGFβ Signaling Increases Direct Conversion of Fibroblasts to Induced Cardiomyocytes,” PLoS One. 9:e89678 (2014); and Mohamed et al., “Chemical Enhancement of In Vitro and In Vivo Direct Cardiac Reprogramming,” Circulation 135:978-995 (2017), all of which are hereby incorporated by reference in their entirety. In addition, as the reprogramming process and the repeated use of transfection reagents can lead to an increased cell death rate, modRNA backbone was designed that reduce apoptosis and stress. Strelow et al. have shown that Acid Ceramidase (AC) overexpression protects cells from elevated cell death and curtails cellular stress. Strelow et al., “Overexpression of Acid Ceramidase Protects From Tumor Necrosis Factor-Induced Cell Death,” J. Exp. Med. 192:601-12 (2000), which is hereby incorporated by reference in its entirety. Therefore, as can be seen in FIG. 10D, non-treated non-CMs were compared with groups treated with either GMT only or GMT with Hand2 or AC (GMTH or GMTHA) modRNA cocktails together with small molecules (SMI) that inhibit TGFβ and WNT pathways (SB431542 and XAV939, respectively) or groups treated with GMTHA with gene modRNA that inhibits TGFβ (Dominant negative (DN) of TGFβ or CCN5) and WNT pathways (DN-Wnt8 or Wnt5a). Groups treated with either GMT, GMTH or GMTHA modRNA cocktail together with SB431542 and XAV939 show cardiac reprogramming activity 14 days after first transfection, as demonstrated by upregulated troponin T (cTNT) and αMHC (FIGS. 10E and 10F). Notably, GMTHA produced the highest reprogramming rate: 48% CM-like cells (αMHC⁺ and αActinin⁺ cells, FIGS. 10G and 10H).

Moreover, it is shown that the reprogramming helper small molecules SB431542 and XAV939 can both be replaced by reprogramming helper modRNA (DN-TGFβ or CCN5 and DN-Wnt8 or Wnt5a modRNA, respectively) without losing reprogramming efficiency (FIGS. 10I-10L). This data suggest that 7 genes (7G) modRNA cocktail (GMTHA with DN-TGFβ and DN-Wnt8) significantly elevates cTNT and αMHC (FIGS. 10I and 10J), resulting in 57% CM-like cells 14 days after first transfections in vitro (FIGS. 10K and 10L). In addition, the collagen and fibronectin gene expression levels in reprogramed CM-like cells were significantly lower than in untreated non-CMs in vitro (FIG. 10M). Importantly, repeated transfection for 28 days but not 21 led to the formation of a small number of contracting CM-like cells with mature sarcomere structures and complete cardiac reprogramming (FIGS. 10N-10P).

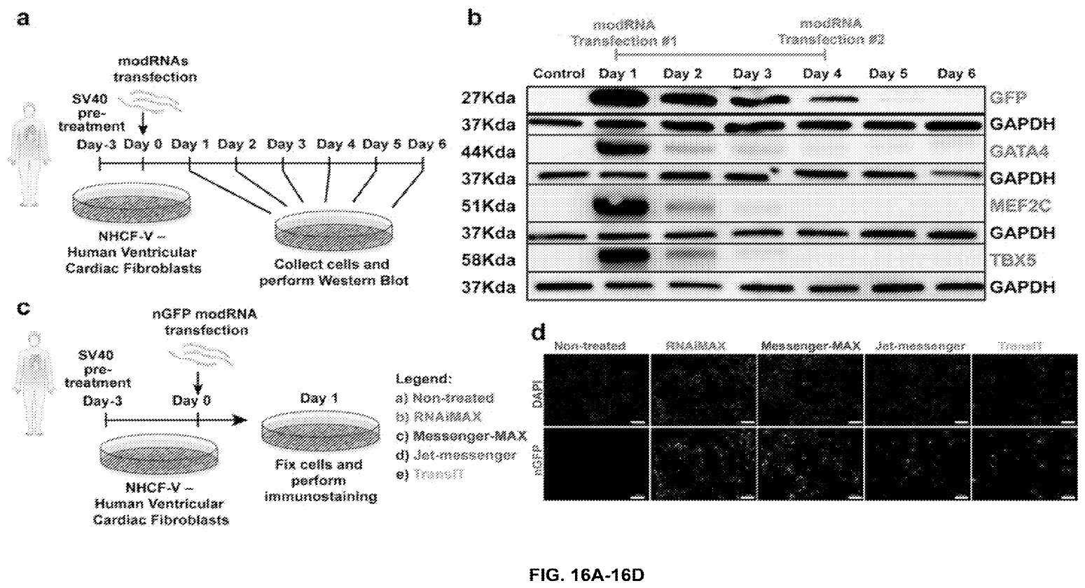

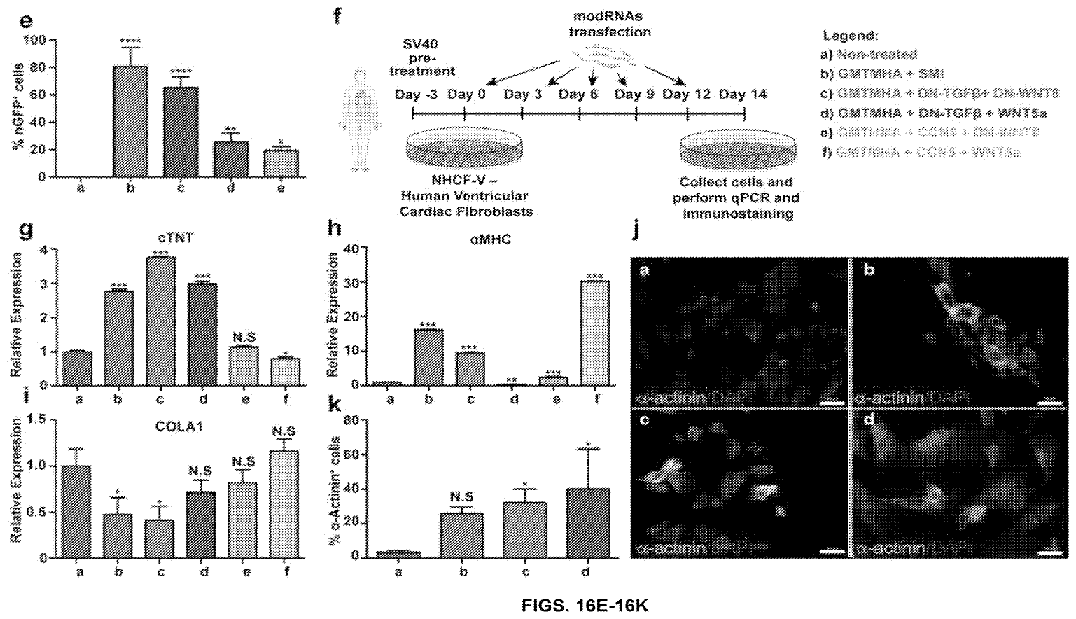

Next, it was assessed if 7G modRNA induces cardiac reprogramming in human ventricle cardiac fibroblasts. First, protein expression analysis was conducted to confirm that GMT modRNA kinetics are similar to those in mouse non-CMs (FIGS. 16A-16B). Different transfection reagents were investigated and it was found, again similar to mouse non-CMs, that the RNAiMAX transfection reagent gave the highest (˜80%) transfection efficiency in comparison to other commercially available transfection reagents (FIGS. 16C-16E). Since previous reports indicate that SV40 pretreatment (Fu et al., “Direct Reprogramming of Human Fibroblasts Toward a Cardiomyocyte-Like State,” Stem Cell Reports 1:235-47 (2013); Nam et al. “Reprogramming of Human Fibroblasts Toward a Cardiac Fate,” Proc. Natl. Acad. Sci. USA 110:5588-93 (2013); Singh et al., “MiR-590 Promotes Transdifferentiation of Porcine and Human Fibroblasts Toward a Cardiomyocyte-Like Fate by Directly Repressing Specificity Protein 1,” J. Am. Heart Assoc. 5 (2016); and Wada et al., “Induction of Human Cardiomyocyte-Like Cells From Fibroblasts by Defined Factors,” Proc. Natl. Acad. Sci. USA. 110:12667-72 (2013), all of which are hereby incorporated by reference in their entirety) and Myocd addition (Fu et al., “Direct Reprogramming of Human Fibroblasts Toward a Cardiomyocyte-Like State,” Stem Cell Reports 1:235-47 (2013); Nam et al. “Reprogramming of Human Fibroblasts Toward a Cardiac Fate,” Proc. Natl. Acad. Sci. USA 110:5588-93 (2013); Wada et al., “Induction of Human Cardiomyocyte-Like Cells From Fibroblasts by Defined Factors,” Proc. Natl. Acad. Sci. USA. 110:12667-72 (2013), all of which are hereby incorporated by reference in their entirety) lead to superior cardiac reprogramming, human ventricle cardiac fibroblasts were pretreated with SV40 modRNA 3 days prior to transfection with or without 6 genes modRNA (GMT+Myocd+Hand2+AC (GMTMHA)) along with reprogramming helper small molecules SB431542 and XAV939 or different genes modRNA that can inhibit TGFβ and WNT pathways (FIG. 16F). These in vitro results show that GMTMHA+DN-TGFβ and DN-WNT8 or GMTMHA+DN-TGFβ and WNT5a modRNA cocktails significantly increased cTNT and αMHC (FIGS. 16G-16H), reduced collagen expression (FIG. 16I) and resulted in 30-40% CM-like cells 14 days after first cardiac reprogramming transfection (FIGS. 16J and 16K).

### Example 5—Screening of Additional Genes to Improve the Reprogramming Efficiency of 7G

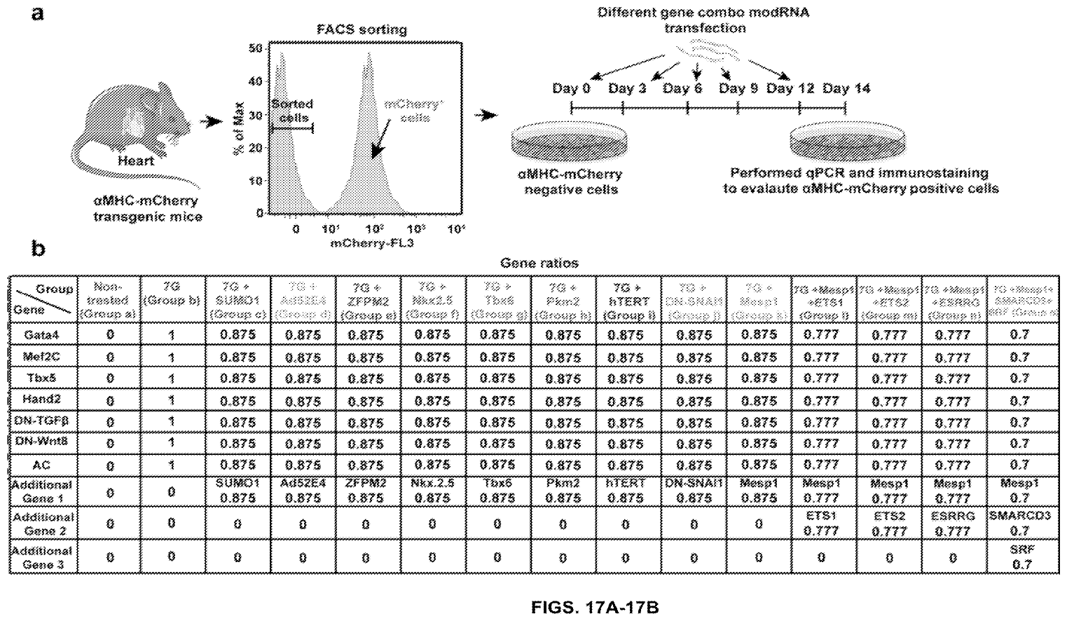

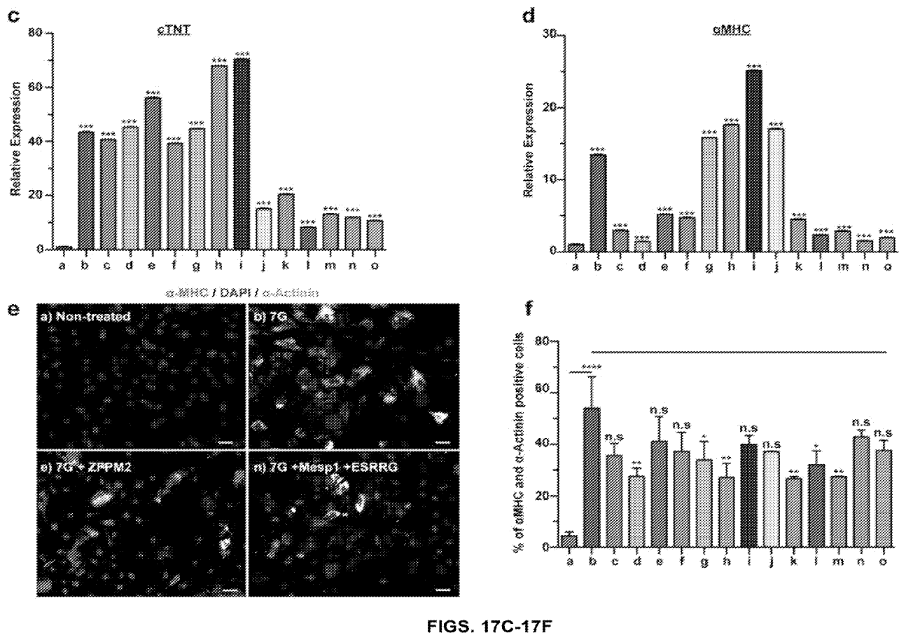

As 7G modRNA can lead to efficient cardiac reprogramming in vitro, it was next evaluated if adding one or several selected candidate genes can enhance 7G cardiac reprogramming. Genes with the potential to increase cardiac reprogramming were pre-selected (Zfpm2 (Fu et al., “Direct Reprogramming of Human Fibroblasts Toward a Cardiomyocyte-Like State,” Stem Cell Reports 1:235-47 (2013), which is hereby incorporated by reference in its entirety), Tbx6 (Sadahiro et al., “Tbx6 Induces Nascent Mesoderm from Pluripotent Stem Cells and Temporally Controls Cardiac versus Somite Lineage Diversification,” Cell Stem Cell. 23:382-395 e5 (2018), which is hereby incorporated by reference in its entirety), DN-SNAI1 (Muraoka et al., “MiR-133 Promotes Cardiac Reprogramming by Directly Repressing Snail and Silencing Fibroblast Signatures,” EMBO J. 33:1565-81 (2014), which is hereby incorporated by reference in its entirety), Mesp1 (Christoforou et al., “Transcription Factors MYOCD, SRF, Mesp1 and SMARCD3 Enhance the Cardio-Inducing Effect of GATA4, TBX5, and MEF2C During Direct Cellular Reprogramming,” PLoS One 8:e63577 (2013), which is hereby incorporated by reference in its entirety), Ets1 (Islas et al., “Transcription Factors ETS2 and MESP1 Transdifferentiate Human Dermal Fibroblasts Into Cardiac Progenitors,” Proc. Natl. Acad. Sci. USA 109:13016-21 (2012), which is hereby incorporated by reference in its entirety), Ets2 (Islas et al., “Transcription Factors ETS2 and MESP1 Transdifferentiate Human Dermal Fibroblasts Into Cardiac Progenitors,” Proc. Natl. Acad. Sci. USA 109:13016-21 (2012), which is hereby incorporated by reference in its entirety), Esrrg (Fu et al., “Direct Reprogramming of Human Fibroblasts Toward a Cardiomyocyte-Like State,” Stem Cell Reports 1:235-47 (2013), which is hereby incorporated by reference in its entirety), SMarcd3 (Christoforou et al., “Transcription Factors MYOCD, SRF, Mesp1 and SMARCD3 Enhance the Cardio-Inducing Effect of GATA4, TBX5, and MEF2C During Direct Cellular Reprogramming,” PLoS One 8:e63577 (2013), which is hereby incorporated by reference in its entirety) or Srf (Christoforou et al., “Transcription Factors MYOCD, SRF, Mesp1 and SMARCD3 Enhance the Cardio-Inducing Effect of GATA4, TBX5, and MEF2C During Direct Cellular Reprogramming,” PLoS One 8:e63577 (2013), which is hereby incorporated by reference in its entirety) or their known function in choreographing cardiac contractility (SUMO1), cell survival (Ad52E4), neonatal cardiac metabolism (Pkm2) or telomere size (hTERT). While all 7G modRNA cocktails with additional candidate modRNA genes led to significant cardiac reprogramming (FIGS. 17A-17D), none of them gave similar or significantly higher cardiac reprogramming activity than 7G alone (FIGS. 17E-17F).

### Example 6-7G modRNA Cocktail with Equal Ratio or Higher Concentrations of Either Hand2 or Mef2c Yields High Cardiac Reprogramming Activity in Mouse Non-CM In Vitro

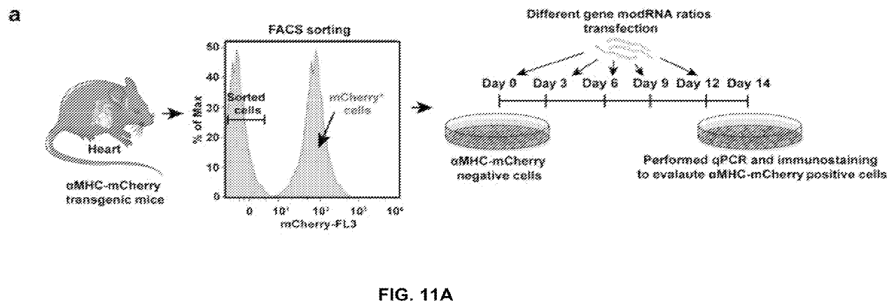

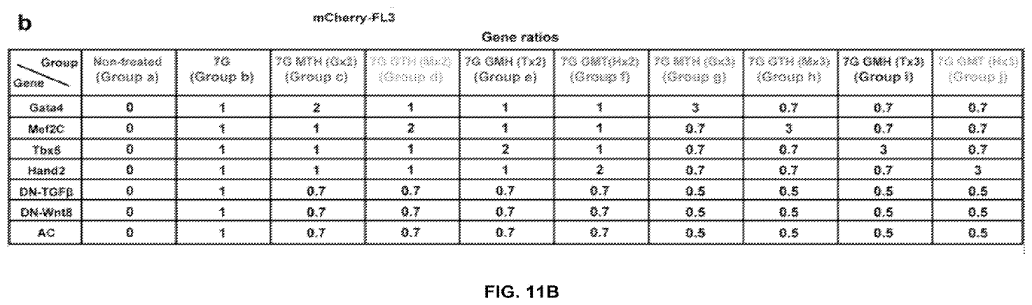

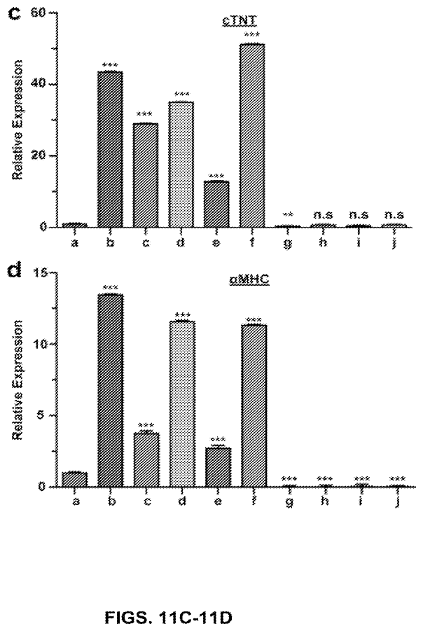

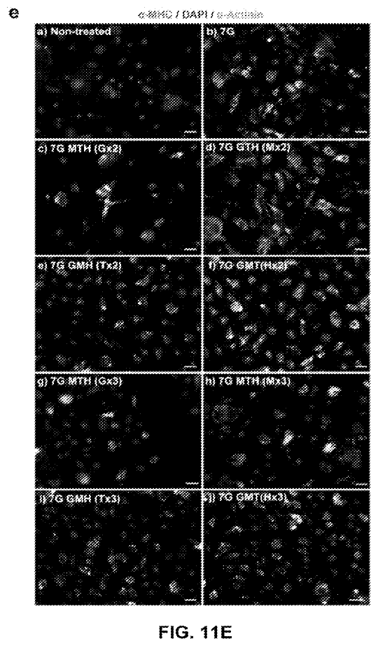

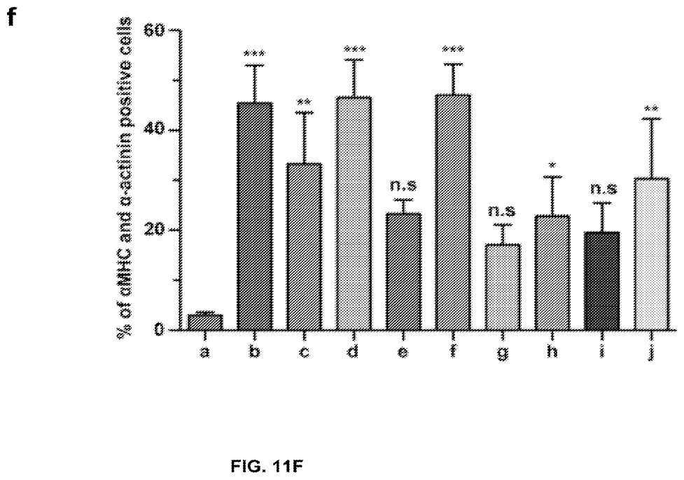

One advantage of modRNA gene delivery methods over viral is the capability to control the amount of mRNA being delivered into the cells. Therefore, the potential of different ratios of reprogramming gene modRNA (GMTH) in the 7G modRNA cocktail in driving cardiac reprogramming were interrogated (FIGS. 11A-11B). It is shown in FIGS. 11A-11F that double but not triple concentrations of Hand2 or Mef2C, compared to TBX5 or GATA4, lead to similar cardiac reprogramming activity as 7G, in vitro (FIGS. 11E-11F). Thus, it is concluded that either 7G modRNA cocktail or enhanced Hand2 7G modRNA cocktail (7G GMT (HX2)) or enhanced Mef2c 7G modRNA cocktail (7G G(MX2)TH) has ˜50% cardiac reprogramming efficiency 14 days post transfections, in vitro.

### Example 7-7G or 7G GMT (HX2) modRNA Cocktails Improve Outcomes after MI

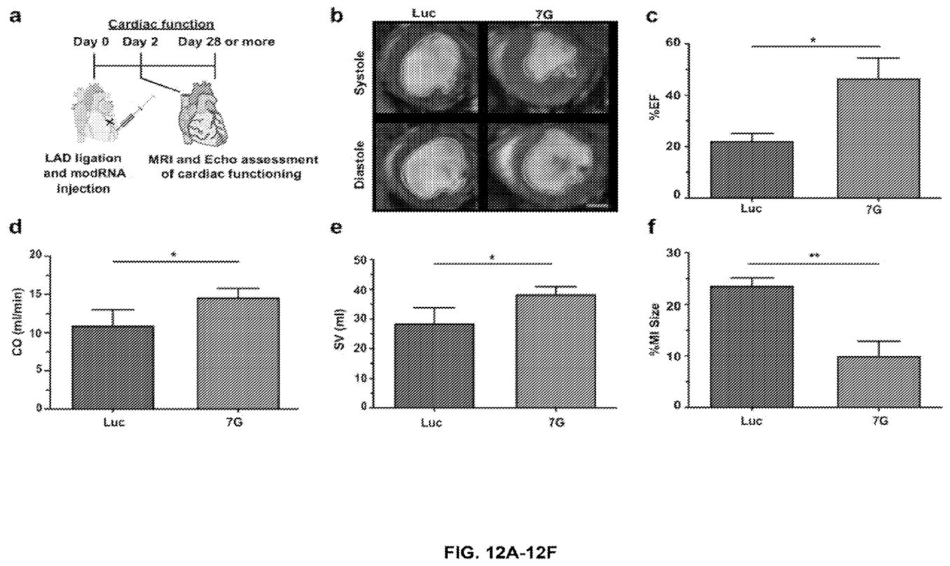

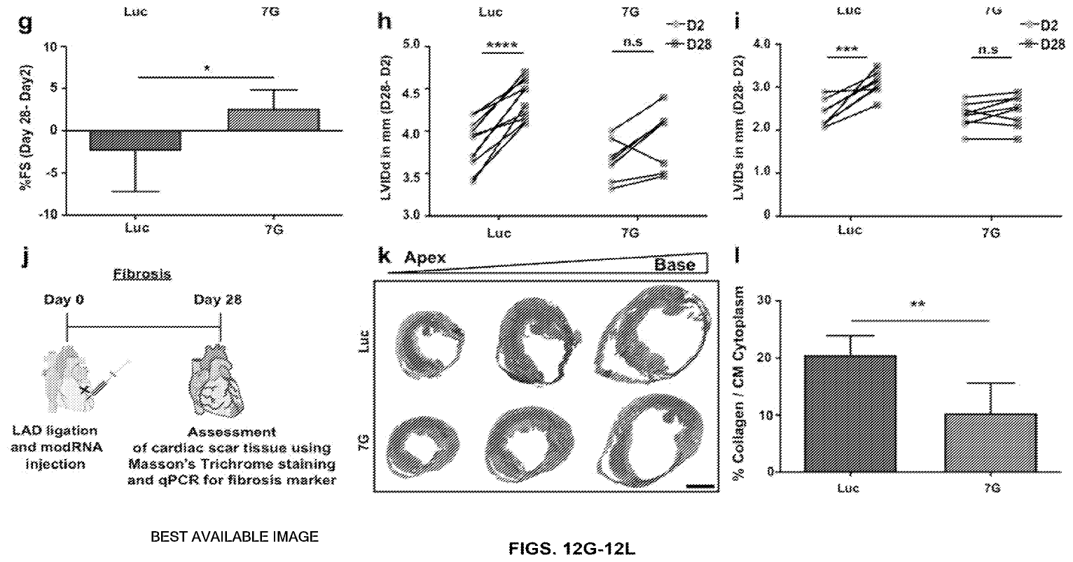

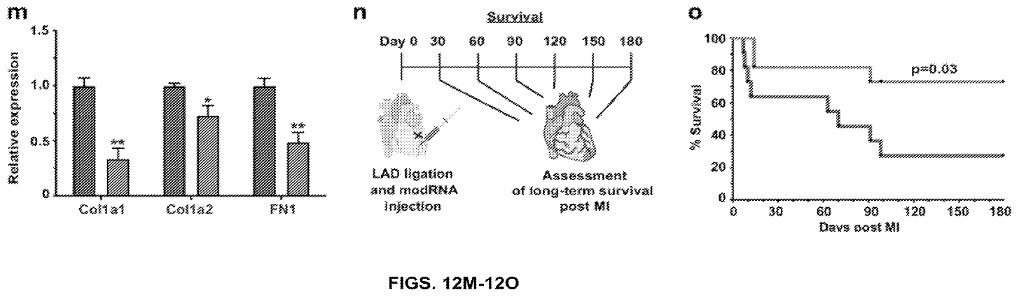

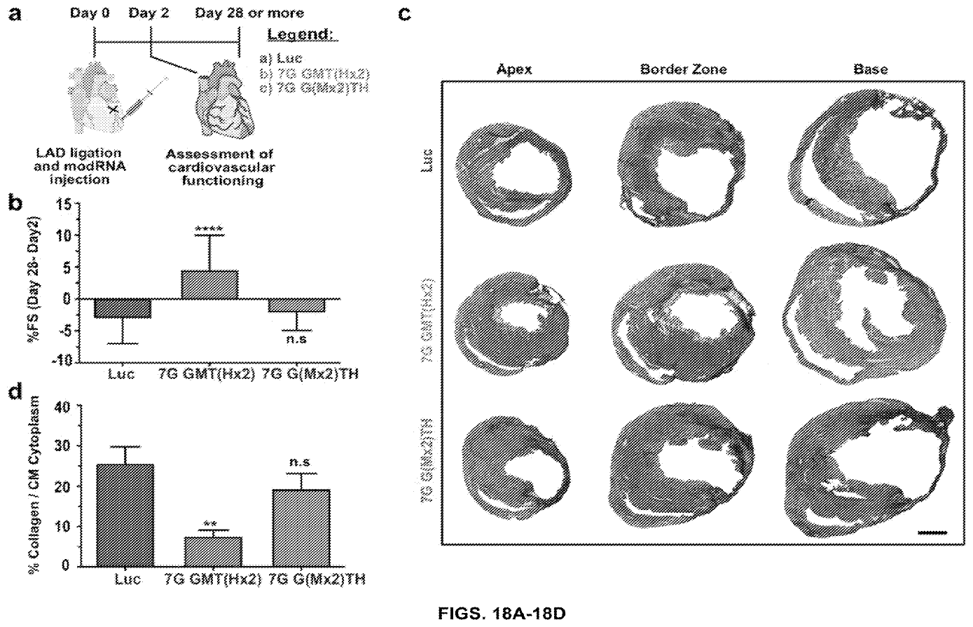

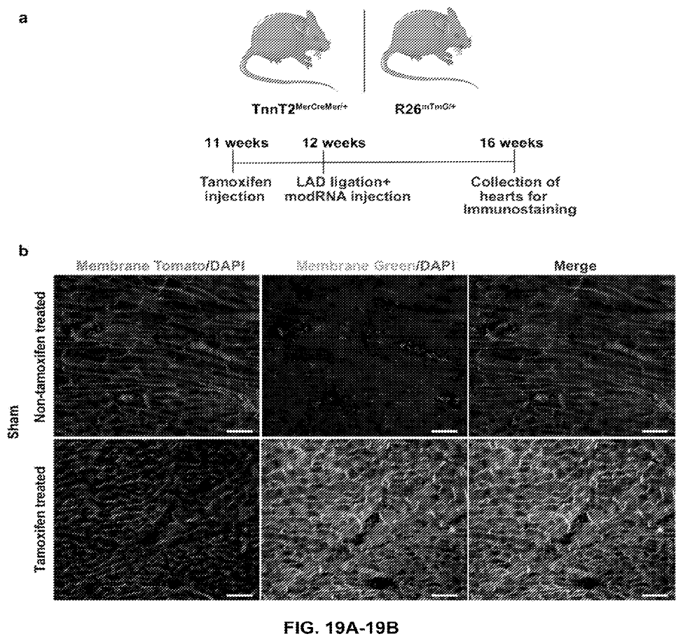

In addition to evaluating cardiac reprogramming in vitro, 7G modRNA cocktail's ability to improve functional outcomes after MI was assessed. To do so, a mouse model of MI was used and cardiac function was measured at days 2 and 28 post MI (FIGS. 12A-12O and FIGS. 18A-18D). MRI evaluation 28 days post MI post Luc or 7G modRNA delivery (FIGS. 12B-12F) showed a significant improvement in percentile ejection fraction (% EF, 48% for 7G) in comparison to 22% for control modRNA (luciferase (Luc)); cardiac output (CO, 14% for 7G) in comparison to 11% for control; stroke volume (SV, 38% for 7G in comparison to 28% for control) and MI size (10% for 7G and in comparison to 24% for control). To confirm that these improvements are not due to differences in MI induction, % fractioning shortening (% FS) and left ventricular internal dimension (LVID) of both end-diastolic (LVIDd) and end-systolic (LVIDs) was measured at days 2 and 28 post MI and treatment with different modRNA cocktails. Percent FS was significantly higher (2.5% for 7G in comparison to −2.4% for control (FIG. 12G). Moreover, while both LVIDd and LVIDs differ significantly between day 2 and day 28 in the control group (FIGS. 12H and 12I), these differences are not significant when 7G modRNA cocktails was delivered. Also evaluated was cardiac scar formation and the expression of extracellular markers like collagen and fibronectin 28 days post MI and different treatments (FIGS. 12J-12M). It is shown that cardiac scars in mice treated with 7G modRNA cocktail was significantly smaller (10%) in comparison to 21% for control (FIGS. 3J-3L) and had lower expression of both collagen and fibronectin (FIG. 3M).

Importantly, 7G with higher concentration of Mef2c (7G G(Mx2)TH did not improve cardiac scarring or cardiac function in comparison to control (FIGS. 18A-18D), while increased Hand2 (7G GMT (HX2) modRNA) showed a similar beneficial effect as 7G modRNA. This finding indicates that in vivo higher concentration of Hand2, but not Mef2c had a beneficial effect on outcome after MI. Lastly, it is shown that 7G modRNA cocktail significantly improved 6-months mouse survival post MI (FIGS. 12N and 12O).

### Example 8-7G modRNA Cocktails Upregulate Angiogenic Paracrine Factors Secretion in the LV and Promote Cardiovascular Regeneration Post MI

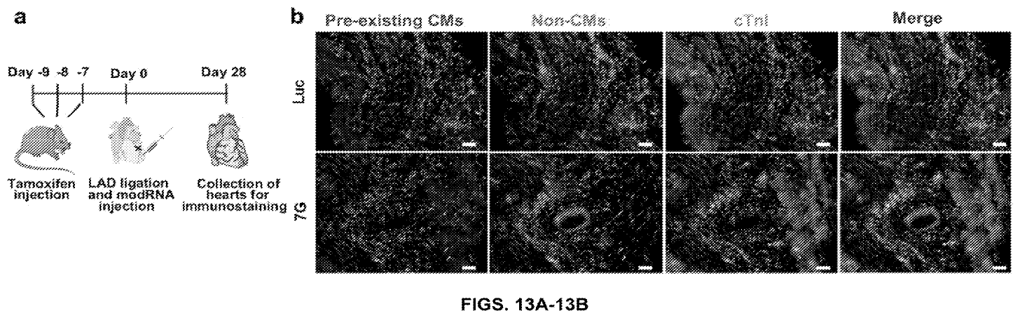

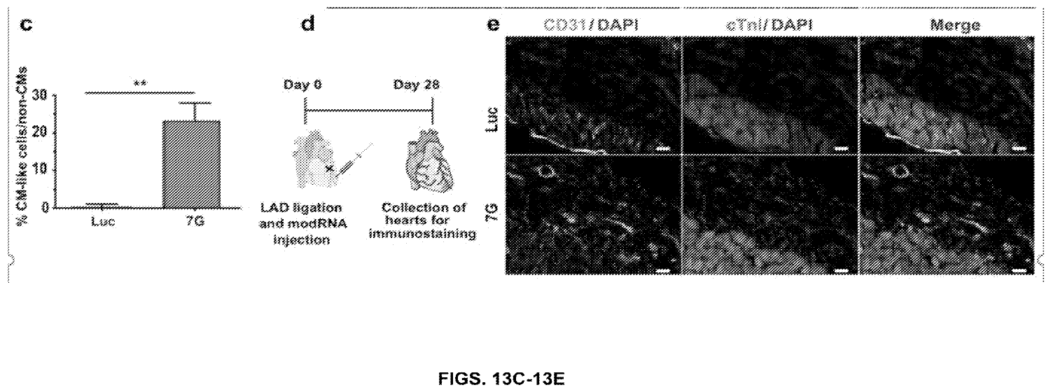

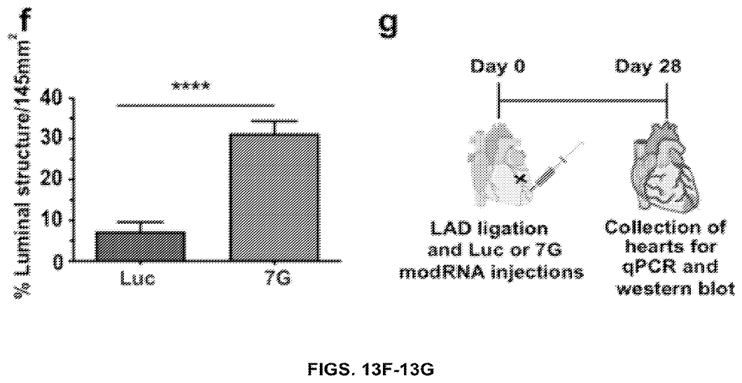

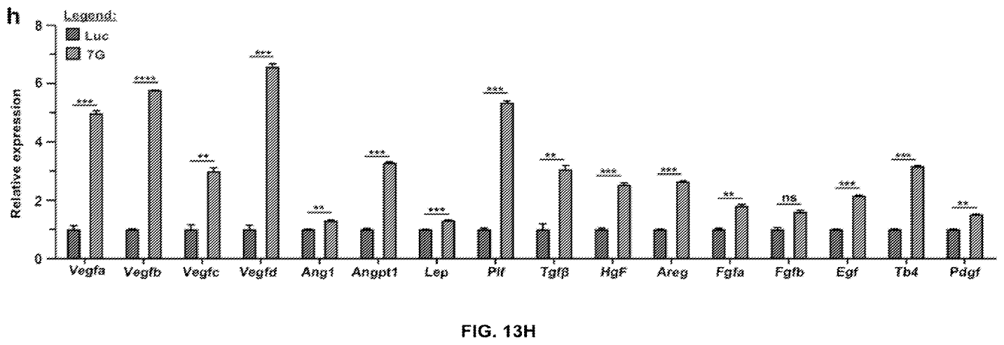

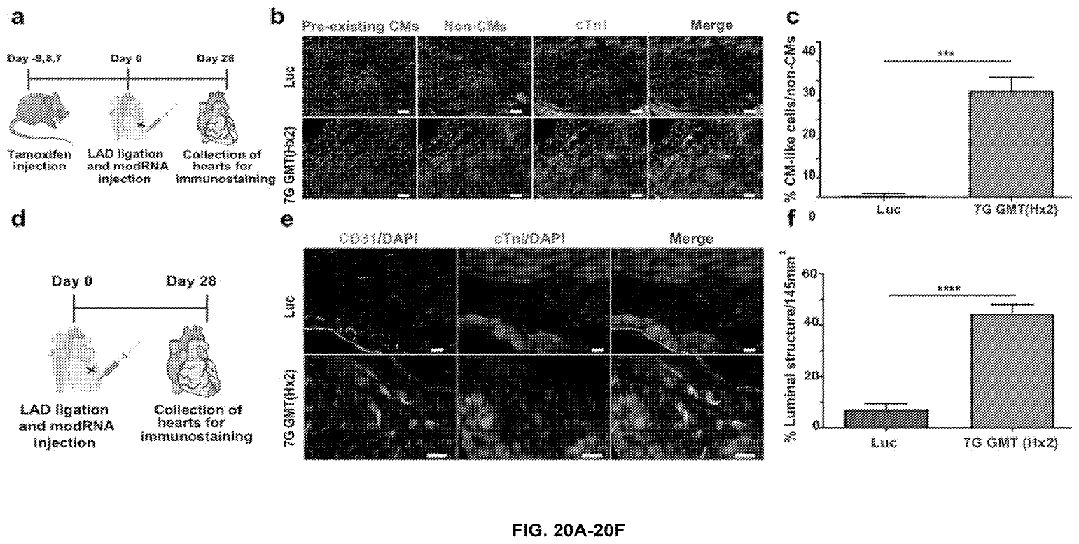

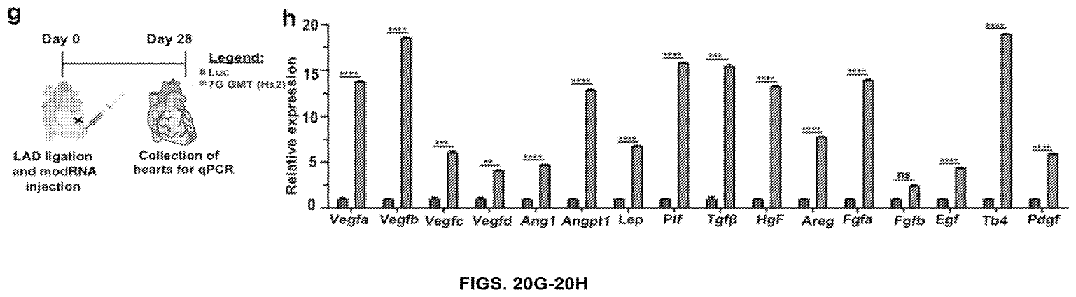

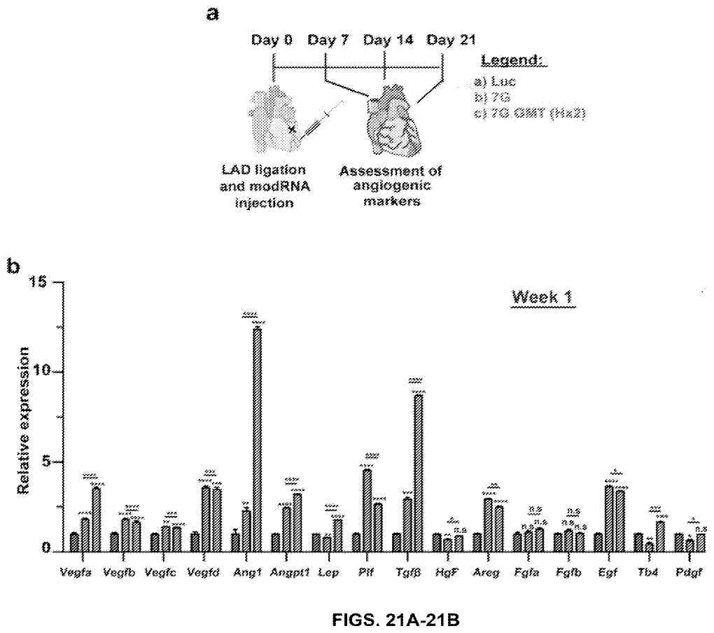

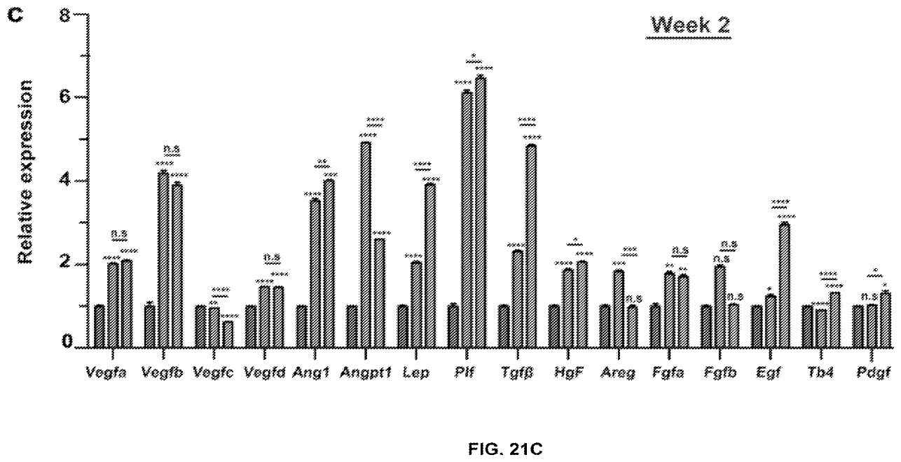

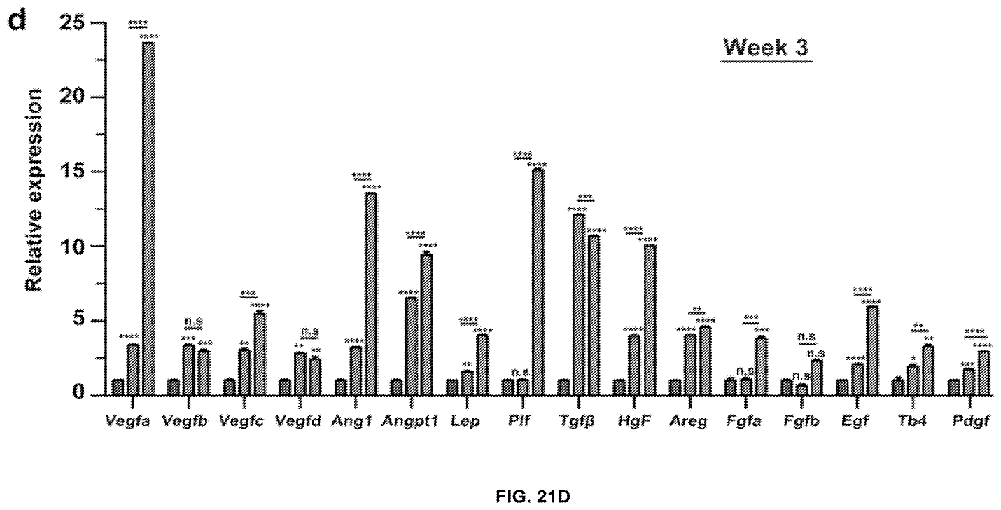

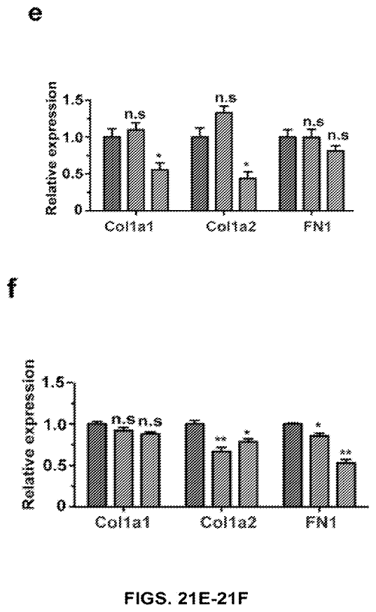

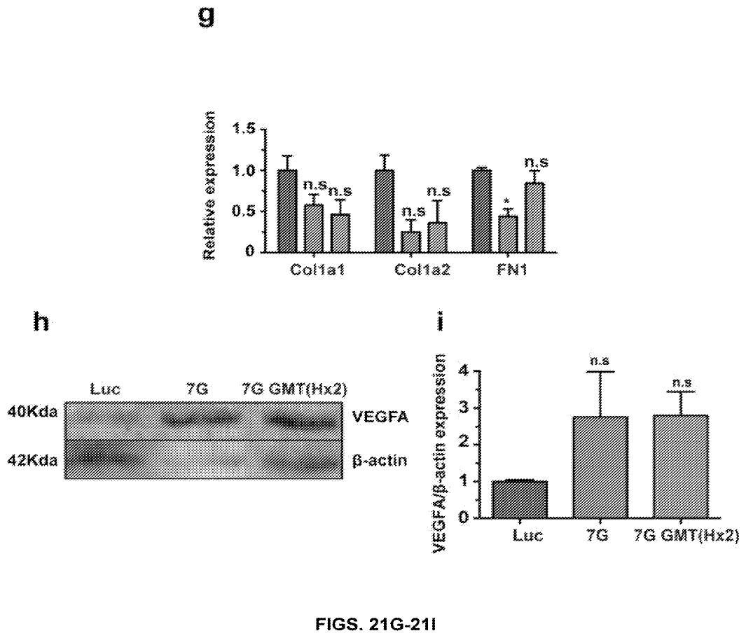

As 7G modRNA cocktails promote CM-like cell formation in vitro and improve post-MI outcomes, their ability to induce CM-like cells in vivo was evaluated. To this end, a lineage-tracing model (TnnT^{Cre/mTmG} mice, FIGS. 19A-19B) was used that pre-labels all preexisting CMs in the heart in green (GFP), while all non-CMs are labeled in red (tdTomato⁺). It is shown that 4 weeks post MI and delivery of 7G modRNA cocktails into the LV, there are 24% CM-like cells (tdTomato⁺ and cTnI⁺ cells) of total non-CMs (tdTomato⁺ cells) (FIGS. 13A-13C). Notably, these CM-like cells lacked a mature sarcomere structure and were much smaller than preexisting mature CMs (FIG. 13B). It was very unlikely that the immature, partial reprogrammed, CM-like cells formed by 7G modRNA cocktail can contribute to cardiac muscle regeneration. Therefore, it was hypothesized that 7G modRNA cocktail's beneficial mechanism of action may occur via paracrine effect to induce cardiovascular regeneration post MI. To test this hypothesis, LV capillary density was evaluated 28 days post MI and delivery of different modRNAs. It is shown that 7G modRNA cocktail significantly promoted capillary density in the LV in compare to control (FIGS. 13D-13F). To evaluate the role of 7G modRNA cocktails angiogenesis factor secretion, qPCR was used to measure gene expression of 16 pre-selected key angiogenic factors that are known for their ability to increase blood vessel formation and maturation. Vegfa (Haro et al., “Vascular Endothelial Growth Factor (VEGF)-Induced Angiogenesis in Herniated Disc Resorption,” J. Orthop. Res. 20:409-15 (2002), which is hereby incorporated by reference in its entirety), Vegfb (Robciuc et al. “VEGFB/VEGFR1-Induced Expansion of Adipose Vasculature Counteracts Obesity and Related Metabolic Complications,” Cell Metab. 23, 712-24 (2016), which is hereby incorporated by reference in its entirety), Vegfc (Cao et al., “Vascular Endothelial Growth Factor C Induces Angiogenesis In Vivo,” Proc. Natl. Acad. Sci. USA 95:14389-94 (1998), which is hereby incorporated by reference in its entirety), Vegfd (Rissanen et al., “VEGF-D is the Strongest Angiogenic and Lymphangiogenic Effector Among VEGFs Delivered Into Skeletal Muscle Via Adenoviruses,” Circ. Res. 92:1098-106 (2003), which is hereby incorporated by reference in its entirety), Ang1 (Koblizek et al., “Angiopoietin-1 Induces Sprouting Angiogenesis In Vitro,” Curr. Biol. 8:529-32 (1998), which is hereby incorporated by reference in its entirety), Angpt1 (Rosa et al., “The Angiogenic Factor Angiopoietin-1 is a Proneurogenic Peptide on Subventricular Zone Stem/progenitor Cells,” J. Neurosci. 30:4573-84 (2010), which is hereby incorporated by reference in its entirety), Lep (Zhou et al., “Leptin Pro-Angiogenic Signature in Breast Cancer is Linked to IL-1 Signalling,” Br. J. Cancer 104:128-37 (2011), which is hereby incorporated by reference in its entirety), Plf (Toft et al., “Reactivation of Proliferin Gene Expression is Associated With Increased Angiogenesis in a Cell Culture Model of Fibrosarcoma Tumor Progression,” Proc. Natl. Acad. Sci. USA 98:13055-9 (2001), which is hereby incorporated by reference in its entirety), Tgfβ (Vinals et al., “Transforming Growth Factor Beta1 (TGF-beta1) Promotes Endothelial Cell Survival During In Vitro Angiogenesis Via an Autocrine Mechanism Implicating TGF-alpha Signaling,” Mol. Cell Biol. 21:7218-30 (2001), which is hereby incorporated by reference in its entirety), Hgf (Kaga et al., “Hepatocyte Growth Factor Stimulated Angiogenesis Without Inflammation: Differential Actions Between Hepatocyte Growth Factor, Vascular Endothelial Growth Factor and Basic Fibroblast Growth Factor,” Vascul. Pharmacol. 57:3-9 (2012), which is hereby incorporated by reference in its entirety), Areg (Wang et al., “Amphiregulin Enhances VEGF-A Production in Human Chondrosarcoma Cells and Promotes Angiogenesis by Inhibiting miR-206 via FAK/c-Src/PKCδ Pathway,” Cancer Lett. 385:261-270 (2017), which is hereby incorporated by reference in its entirety), Fgfa (Murakami et al., “Fibroblast Growth Factor Regulation of Neovascularization,” Curr. Opin. Hematol. 15:215-20 (2008), which is hereby incorporated by reference in its entirety), Fgfb (Murakami et al., “Fibroblast Growth Factor Regulation of Neovascularization,” Curr. Opin. Hematol. 15:215-20 (2008), which is hereby incorporated by reference in its entirety), Egf (Mehta et al., “HB-EGF Promotes Angiogenesis in Endothelial Cells via PI3-kinase and MAPK Signaling Pathways,” Growth Factors 25:253-63 (2007), which is hereby incorporated by reference in its entirety), Tb4 (Lv et al., “Thymosin beta4 Induces Angiogenesis Through Notch Signaling in Endothelial cells,” Mol. Cell Biochem. 381:283-90 (2013), which is hereby incorporated by reference in its entirety) and Pdgf (Xie et al., “PDGF-BB Secreted by Preosteoclasts Induces Angiogenesis During Coupling with Osteogenesis,” Nat. Med. 20:1270-8 (2014), which is hereby incorporated by reference in its entirety) was assessed one, two, three or four weeks post MI and delivery of 7G modRNA cocktail or Luc control modRNA (FIGS. 13G, 13H). There was a significant upregulation of 15 out of 16 (FGF-2 was the exception) angiogenic factors from 1 week to 4 weeks post MI. Similar results were seen when 7G GMT (Hx2) modRNA cocktail was delivered post MI (FIGS. 20A-20H). This indicated that 7G and 7G GMT (Hx2) modRNA cocktail delivery might have induced an angiogenic mechanism after in the MI setting. Moreover, this effect started 1 week after modRNA delivery (FIGS. 21A-21G) and lasted for at least 4 week, long after modRNA translation was ceased (˜day 10).

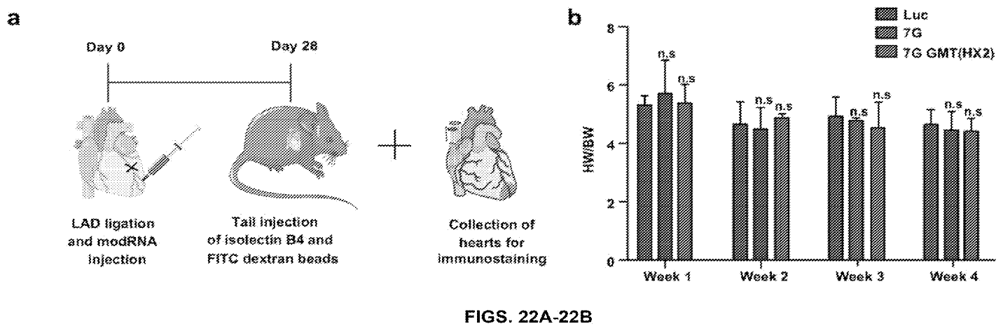

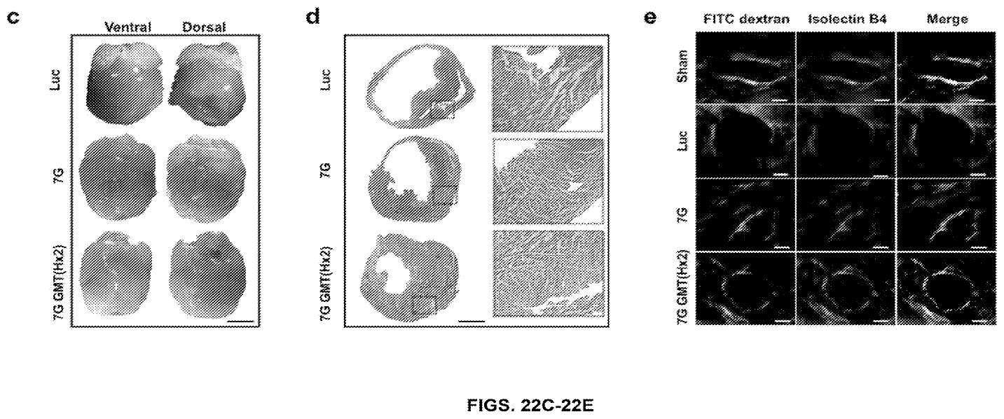

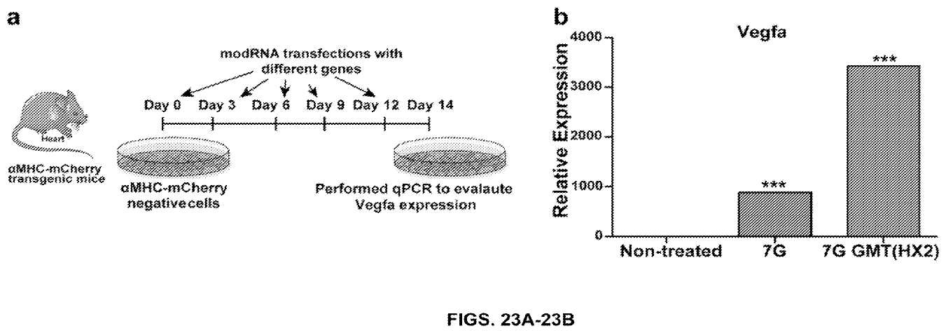

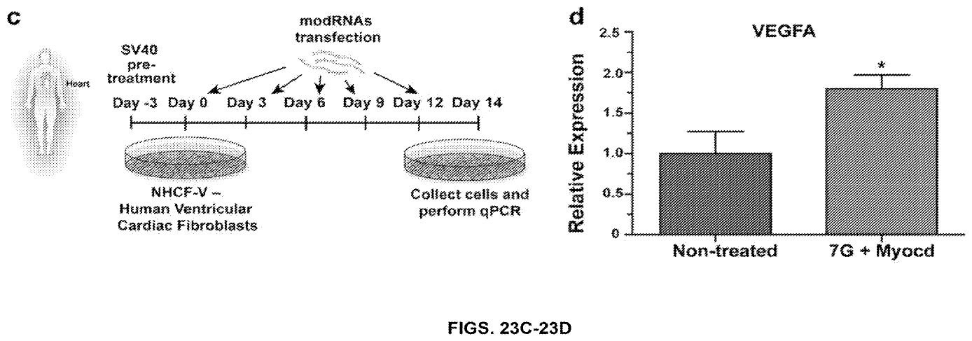

Since Vegfa protein is a key regulator of cardiac vascularization (Zangi et al., “Modified mRNA Directs the Fate of Heart Progenitor Cells and Induces Vascular Regeneration After Myocardial Infarction,” Nat. Biotechnol. 31:898-907 (2013) and Kikuchi et al., “An Antiangiogenic Isoform of VEGF-A Contributes to Impaired Vascularization in Peripheral Artery Disease,” Nat. Med. 20:1464-71 (2014), both of which are hereby incorporated by reference in their entirety), western blot analysis was used to evaluate Vegfa levels 21 or 28 days post MI and different modRNA treatments. It was shown that Vegfa levels are significantly higher in the LV 28 days but not 21 days post MI (FIGS. 13G-13J and FIGS. 21H and 21I). In addition, qPCR evaluation of LV at different time points post MI with various treatments indicated that 7G or 7G GMT (Hx2) modRNA cocktails reduce, especially in week 2 post MI, the levels of Col1a2 and fibronectin (FIGS. 21E-21F). As high unregulated Vegfa levels in the heart post MI can have deleterious side effects like edema and angioma and may lead to leaky blood vessels (Zangi et al., “Modified mRNA Directs the Fate of Heart Progenitor Cells and Induces Vascular Regeneration After Myocardial Infarction,” Nat. Biotechnol. 31:898-907 (2013) and Lee et al., “VEGF Gene Delivery to Myocardium: Deleterious Effects of Unregulated Expression,” Circulation 102:898-901 (2000), both of which are hereby incorporated by reference in their entirety), these factors were evaluated in the heart at different time points post MI and different modRNA cocktail treatments (FIGS. 22A-22E). As can be seen in FIGS. 21A-21G, 7G and 7G GMT (Hx2) modRNA cocktails did not notably change heart weight to body weight ratio (FIG. 22B) or heart size (FIG. 22C) and did not induce angioma formation in the myocardium (FIG. 22D) or leaky vessels (FIG. 22E), as compared to Luc control modRNA 28 days post MI. It was concluded that although both 7G and 7G GMT (HX2) modRNA cocktails raise Vegfa protein levels in the LV after MI (FIGS. 13I, 13J), this elevation is not associated with Vegfa's detrimental effects (FIGS. 22A-22E). In addition, it was determined if cardiac reprogramming of non-CM from mouse or human hearts in vitro could also induce higher Vegfa expression 14 days following transfections with or without modRNA cocktails. Both mouse (FIGS. 23A, 23B) and human (FIGS. 23C, 23D) cardiac reprogramming led to significantly increased Vegfa expression, demonstrating that conversion of non-CM to CM-like cells might turn on a pro-angiogenic program, facilitating this reparative process.

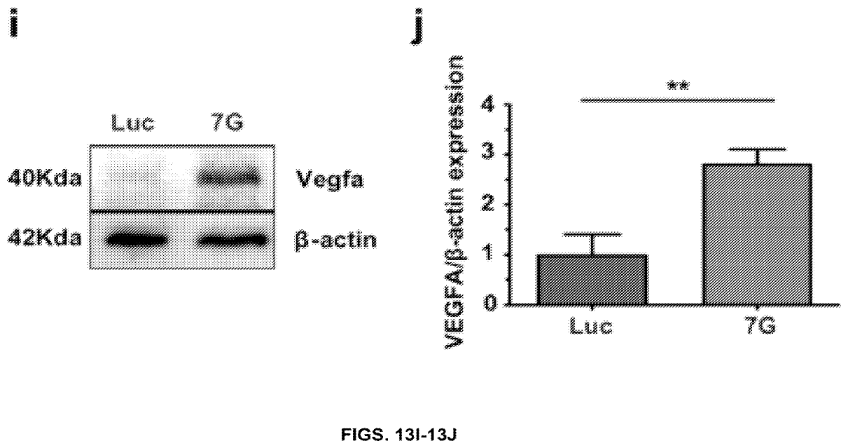

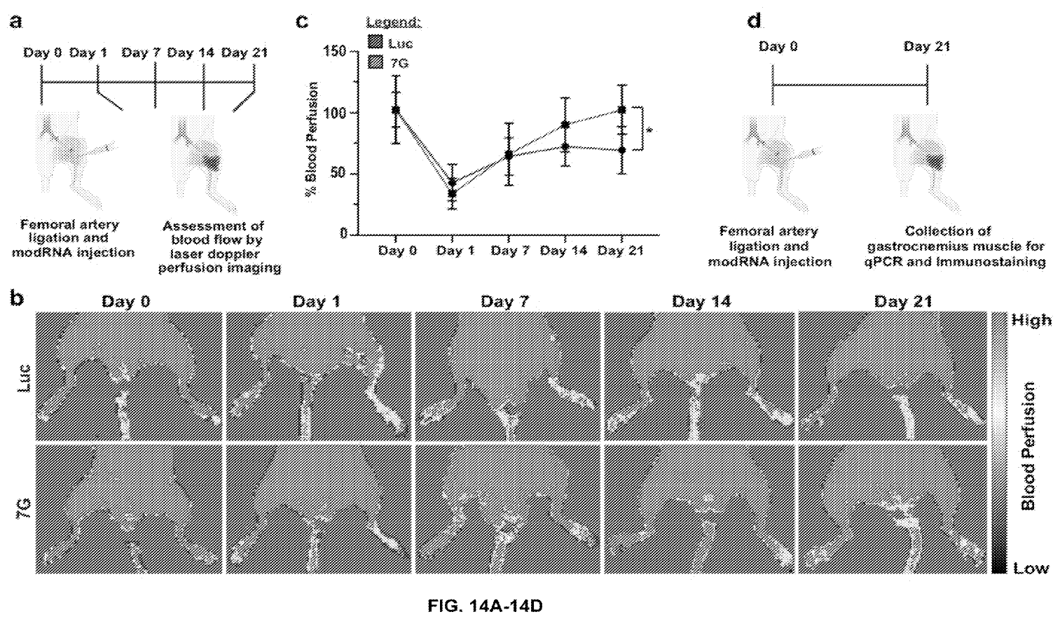

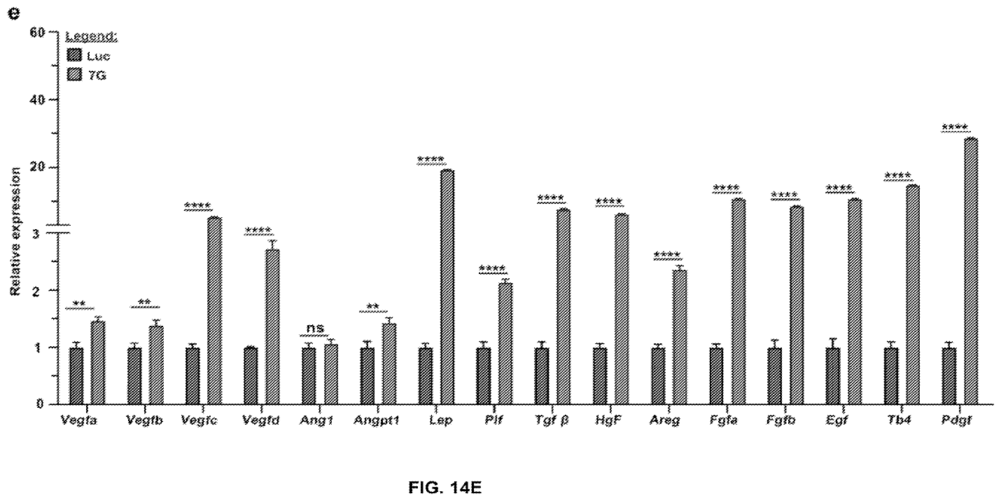

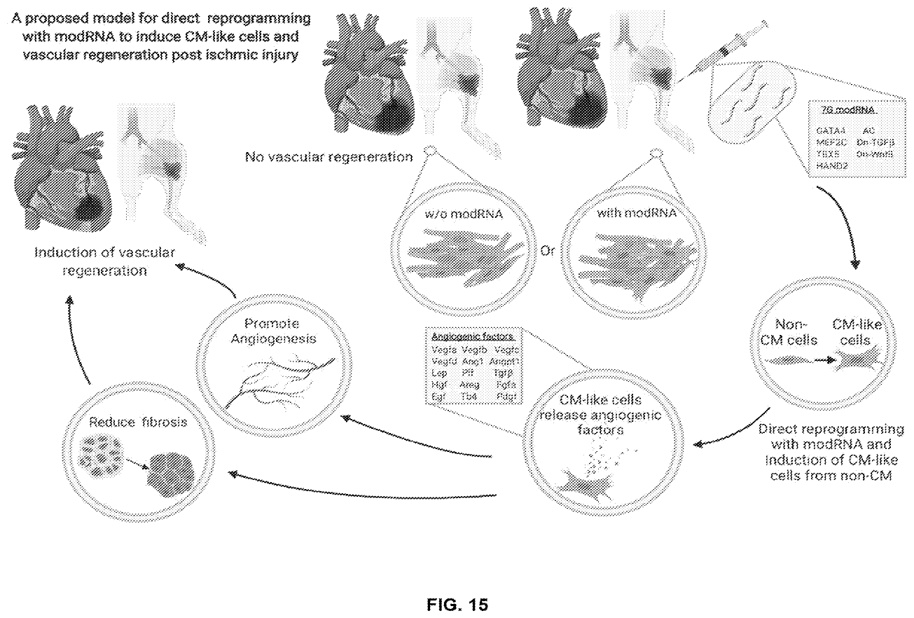

To further evaluate this pro-angiogenic program in non-CMs it was sought to test if injection of 7G modRNA under ischemic condition in a non-cardiac muscle (e.g skeletal muscle) can lead to enhance vascularization post injury. ApoE−/− mouse hindlimb ischemia model was shown previously to use as a preferred model for vascular regeneration in skeletal muscle. Kang et al., “Apolipoprotein E−/− Mice Have Delayed Skeletal Muscle Healing After Hind Limb Ischemia-Reperfusion,” J. Vasc. Surg. 48:701-8 (2008), which is hereby incorporated by reference in its entirety. 7G modRNA cocktail or Luc modRNA was injected immediately post-femoral artery ligation. Laser Doppler perfusion imaging was used to evaluate blood perfusion in the foot region, at day 0, 1, 7, 14 and 21 post injury (FIG. 14A). The data show that 7G modRNA cocktail significantly promote improvement in blood perfusion in comparison to Luc modRNA (FIGS. 14B and 14C). Also, it is shown that key angiogenic factors expressions were significantly unregulated, similar to cardiac tissue, post muscle ischemic injury and delivery of 7G modRNA cocktail in compare to Luc control modRNA. In conclusion, this data shows that 7G modRNA cocktail induce pro-angiogenic program in cells in cardiac and skeletal muscles (FIG. 15).

### Example 9—Discussion

The mammalian heart contains ˜50% CMs, while the rest are non-CMs. Post cardiac ischemic injury, large numbers of CMs die and are replaced by non-CM cell types. One approach to overcome this imbalance in CM ratio is to reprogram non-CMs to CMs and thereby generate de novo CMs. The main obstacles to using such reprogramming for cardiac repair are the low efficacy of cardiac reprogramming and the possibly detrimental side effects using viral delivery methods and small molecules. In this disclosure, it is shown that delivering reprogramming genes and helper genes via modRNA technology eliminates the need for viral transfection or small molecules and leads to high numbers of CM-like cells in vitro and in vivo. To date, the highest reprogramming efficiency reported 14 days post transfection yielded ˜30% CM-like cells. Mohamed et al., “Chemical Enhancement of In Vitro and In Vivo Direct Cardiac Reprogramming,” Circulation 135:978-995 (2017) and Nam et al., “Induction of Diverse Cardiac Cell Types by Reprogramming Fibroblasts With Cardiac Transcription Factors,” Development 141:4267-78 (2014), both of which are hereby incorporated by reference in their entirety. Here, it is demonstrated that using 7G or 7G GMT (Hx2) modRNA cocktails increases reprogramming activity and yields ˜50% CM-like cells in vitro (FIGS. 10A-10P, FIGS. 11A-11F). It is also shown that using 7G+Myocd or GMTMHA+DN-TGFβ and WNT5a modRNA cocktails leads to 30 or 40% reprogramming efficiency in human ventricle cardiac fibroblasts (FIGS. 16A-16J). These results are similar to previous reports of human cardiac reprogramming with GHTM, miR-1 and miR133a. Nam et al. “Reprogramming of Human Fibroblasts Toward a Cardiac Fate,” Proc. Natl. Acad. Sci. USA 110:5588-93 (2013), which is hereby incorporated by reference in its entirety. Additionally, the fact that repeated transfection for 28 days produces mature-like contracting CMs leads to the conclusion that modRNA is an efficient and safe gene delivery system for inducing CM-like cells in vitro (FIGS. 10N-10P). Any additional gene or change was not able to be identified in reprogramming gene modRNA ratios that gave higher reprogramming levels. However, it is shown that elevating any of the cardiac reprogramming genes to three-fold, abolished the reprogramming process (FIGS. 11A-11F). This indicates that each reprogramming gene plays an essential role and that the lack or low levels of any one of them prevents reprogramming.

Using a lineage tracing in vivo model, it is confirmed that modRNA successfully promotes the formation of ˜25% CM-like cells in the LV 28 days post MI and delivery of 7G modRNA cocktail (FIGS. 13A-13C). This is a higher cardiac reprogramming efficiency than the previously published 12% cardiac reprogramming in vivo. Jayawardena et al., “MicroRNA Induced Cardiac Reprogramming In Vivo: Evidence For Mature Cardiac Myocytes and Improved Cardiac Function,” Circ. Res. 116: 418-24 (2015), which is hereby incorporated by reference in its entirety. Nonetheless, no CMs were detected with mature sarcomeres among the CM-like cell population. This could be due to the finding that short-term (˜10 days) use of the modRNA delivery platform was insufficient for effective reprogramming of non-CMs to mature contracting CMs, as was shown previously using viral delivery systems that have long-term expression patterns. Song et al., “Heart Repair by Reprogramming Non-Myocytes With Cardiac Transcription Factors,” Nature 485:599-604 (2012), which is hereby incorporated by reference in its entirety. Even without the formation of de novo adult CM, the protocol led to significantly improved cardiac function, scar size and long-term survival 28 days post MI and delivery of 7G or 7G GMT (HX2) modRNA cocktails (FIGS. 12A-12) and FIGS. 18A-18D). It was also demonstrated that this improvement is due to induction of angiogenic factors during modRNA partial cardiac reprogramming process (FIGS. 13A-13J, FIGS. 20A-20H, FIGS. 21A-21G). As modRNA gene delivery maintained DNA integrity and has not been shown to induce insertional mutagenesis, there is hope that for clinical gene delivery modRNA will be preferred over lenti- or retro-viral systems. However, these gene transfer viral methods generate small amounts of functional CMs in the LV and are afflicted with serious disadvantageous side effects for their clinical use. Additionally, it is shown that modRNA can eliminate the need for small molecules to inhibit TGFβ and WNT pathways in the reprogramming process. This enabled for the composition of all-modRNA cocktails that can enhance cardiac reprogramming and lead to higher capillary density and cardiovascular regeneration post MI.

Importantly, this modRNA reprogramming cocktails did not lead to aberrant outcomes, such as cardiac angioma or edema (FIGS. 22A-22E). This indicates that modRNA upregulation of secreted angiogenic factors are tightly regulated. Thus, modRNA directed cardiac reprogramming makes no contribution to mature CMs; rather, promotes cardiac protection and cardiovascular regeneration post MI occur via a paracrine effect. In this regard, these findings are similar to previous attempts to deliver angiogenic factors secreting cells (mostly bone marrow-derived cells) to the LV in order to promote cardiovascular regeneration (Gong et al., “Exosomes Derived From SDF1-overexpressing Mesenchymal Stem Cells Inhibit Ischemic Myocardial Cell Apoptosis and Promote Cardiac Endothelial Microvascular Regeneration in Mice With Myocardial Infarction,” J. Cell Physiol. 234:13878-13893 (2019); Leong et al., “Cardiac Stem Cells for Myocardial Regeneration: They Are Not Alone,” Front Cardiovasc Med. 4:47 (2017); Lader et al., “Cardiac Stem Cells for Myocardial Regeneration: Promising But Not Ready For Prime Time,” Curr. Opin. Biotechnol. 47:30-35 (2017); Beltrami et al., “Adult Cardiac Stem Cells are Multipotent and Support Myocardial Regeneration,” Cell 114:763-76 (2003); Yoon et al., “Clonally Expanded Novel Multipotent Stem Cells From Human Bone Marrow Regenerate Myocardium After Myocardial Infarction,” J. Clin. Invest. 115:326-38 (2005), all of which are hereby incorporated by reference in their entirety), which marginal benefits. Thus, the modRNA approach might lay the foundation to improve upon previous ineffective cytokine delivery approaches to ignite cardiac repair. Also, the ability to show that 7G modRNA is capable to promote pro-angiogenic program outside the cardiac tissue (e.g skeletal muscle FIGS. 14A-14E), indicate that 7G, that 4 of the genes (Gata4, Mef2c, Tbx5 and Hand2) are heart developmental genes, may play a role not strictly in cardiac tissue, but rather can promote pro-angiogenic program in non-CMs outside the heart. This data shed light on the beneficial mechanism of action of partial cardiac reprograming that can promote vascular regeneration post ischemic muscle injury. Further, the remarkable performance of the 7G modRNA cocktail is reported in induction of angiogenesis in other ischemic organs beyond heart for which hind limb ischemic model was employed. Although, VEGFA modRNA have shown to enhance blood vessel regeneration in ischemic cardiac or skeletal muscle mouse models before (Zangi et al., “Modified mRNA Directs the Fate of Heart Progenitor Cells and Induces Vascular Regeneration After Myocardial Infarction,” Nat. Biotechnol. 31:898-907 (2013) and Witman et al., “Cell-Mediated Delivery of VEGF Modified mRNA Enhances Blood Vessel Regeneration and Ameliorates Murine Critical Limb Ischemia,” J. Control Release 310:103-114 (2019), which are hereby incorporated by reference in their entirety), here it is established that 7G cocktail enhance non-CMs to secrete, in physiological levels, angiogenic factors, long after modRNA translation is complete, and induce vascular regeneration in vivo presenting an advanced strategy for treatment of ischemic injuries.

Since 2002, bone marrow-derived cells have been used in many clinical trials to induce vascular regeneration in patients with both acute MI and chronic ischemic heart or chronic critical limb ischemia disease. Leong et al., “Cardiac Stem Cells for Myocardial Regeneration: They Are Not Alone,” Front Cardiovasc Med. 4:47 (2017) and Ponemone et al., “Safety and Effectiveness of Bone Marrow Cell Concentrate in the Treatment of Chronic Critical Limb Ischemia Utilizing a Rapid Point-of-Care System,” Stem Cells Int. 4137626 (2017), which are hereby incorporated by reference in their entirety. Years of clinical trials have shown this treatment is safe and leads to modest improvements in physiologic and anatomic parameters, above and beyond conventional therapy. Leong et al., “Cardiac Stem Cells for Myocardial Regeneration: They Are Not Alone,” Front Cardiovasc Med. 4:47 (2017) and Ponemone et al., “Safety and Effectiveness of Bone Marrow Cell Concentrate in the Treatment of Chronic Critical Limb Ischemia Utilizing a Rapid Point-of-Care System,” Stem Cells Int. 4137626 (2017), which are hereby incorporated by reference in their entirety. However, the fundamental problems with this method are poor survival of the injected cells and the need to increase the number of ex vivo-cultured cells, typically from the same donor. Gong et al., “Exosomes Derived From SDF1-overexpressing Mesenchymal Stem Cells Inhibit Ischemic Myocardial Cell Apoptosis and Promote Cardiac Endothelial Microvascular Regeneration in Mice With Myocardial Infarction,” J. Cell Physiol. 234:13878-13893 (2019); Leong et al., “Cardiac Stem Cells for Myocardial Regeneration: They Are Not Alone,” Front Cardiovasc Med. 4:47 (2017); Lader et al., “Cardiac Stem Cells for Myocardial Regeneration: Promising But Not Ready For Prime Time,” Curr. Opin. Biotechnol. 47:30-35 (2017); Beltrami et al., “Adult Cardiac Stem Cells are Multipotent and Support Myocardial Regeneration,” Cell 114:763-76 (2003); Yoon et al., “Clonally Expanded Novel Multipotent Stem Cells From Human Bone Marrow Regenerate Myocardium After Myocardial Infarction,” J. Clin. Invest. 115:326-38 (2005), all of which are hereby incorporated by reference in their entirety. Therefore, this approach is to use over-the-shelf modRNA cocktails that can convert non-CMs from the scar area to angiogenic factors secreting cells (similar to bone marrow-derived cells) without engraftment issues or the need to culture cells ex vivo.

While it is shown that modRNA angiogenic effect also accrues in human cardiac reprogramming (FIGS. 23A-23D), there is a need to quantify the role of modRNA cocktails in inducing vascular regeneration in large animal models (e.g. swine). Taken together, the experiments in this study reveal the therapeutic angiogenic function of 7G modRNA cocktail post ischemic muscle injury and may encourage others to create different, novel modRNA cocktails for partial or complete reprogramming or other types of multicomponent drug discovery.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A composition comprising molecules of modified mRNA (modRNA) encoding GATA Binding Protein 4 (G), modRNA encoding Myocyte Enhancer Factor 2C (M), modRNA encoding T-box 5 (T), modRNA encoding Heart- and neural crest derivatives-expressed protein 2 (H), modRNA encoding dominant negative transforming growth factor beta (dnT), and modRNA encoding dominant negative Wingless-related integration site 8 (dnW), wherein said molecules of modRNAs are present in said composition in a ratio of G:M:T:H:dnT:dnW.

2. The composition of claim 1, wherein said ratio consists of 1:1:1:1:1:1.

3. The composition of claim 1, wherein said ratio consists of 2:2:2:2:0.7:07.

4. The composition of claim 1, wherein said ratio consists of 2:1:1:1:0.7:0.7.

5. The composition of claim 1, wherein said ratio consists of 1:2:1:1:0.7:0.7.

6. The composition of claim 1, wherein said ratio consists of 1:1:2:1:0.7:0.7.

7. The composition of claim 1, wherein said ratio consists of 1:1:1:2:0.7:0.7.

8. The composition of claim 1, wherein said ratio consists of 1:2:1:2:0.5:0.5.

9. A pharmaceutical composition comprising the composition of claim 1 and a pharmaceutically acceptable carrier.

10. A method for increasing a ratio of a number of cardiomyocytes to a number of non-cardiomyocytes within a population of cells comprising contacting said population of cells with the composition of claim 1.

11. The method of claim 10, wherein said non-cardiomyocytes comprise cardiac fibroblasts.

12. A method for treating cardiac injury comprising administering to a patient in need of such treatment a therapeutically effective amount of a composition of claim 1.

13. The method of claim 12, wherein said cardiac injury comprises myocardial infarction.

14. The method of claim 12, wherein a cause of said cardiac injury comprises reperfusion injury.

15. A method for stimulating vascular regeneration following ischemic damage comprising contacting tissue damaged by ischemic damage with the composition of claim 1.

16. A method for treating stroke comprising administering to a patient in need of said treatment a therapeutically effective amount of a composition of claim 1.

17. A method for enhancing wound healing comprising administering to a patient in need of such enhancement a therapeutically effective amount of a composition of claim 1.

18. A composition comprising molecules of modified mRNA (modRNA) encoding GATA Binding Protein 4 (G), modRNA encoding Myocyte Enhancer Factor 2C (M), modRNA encoding T-box 5 (T), modRNA encoding Heart- and neural crest derivatives-expressed protein 2 (H), modRNA encoding acid ceramidase (A), modRNA encoding dominant negative transforming growth factor beta (dnT), and modRNA encoding dominant negative Wingless-related integration site 8 (dnW), wherein said molecules of modRNAs are present in said composition in a ratio of G:M:T:H:A:dnT:dnW.

19. The composition of claim 18, wherein said ratio consists of 1:1:1:1:1:1:1.

20. The composition of claim 18, wherein said ratio consists of 2:2:2:2:0.7:0.7:0.7.

21. The composition of claim 18, wherein said ratio consists of 2:1:1:1:0.7:0.7:0.7.

22. The composition of claim 18, wherein said ratio consists of 1:2:1:1:0.7:0.7:0.7.

23. The composition of claim 18, wherein said ratio consists of 1:1:2:1:0.7:0.7:0.7.

24. The composition of claim 18, wherein said ratio consists of 1:1:1:2:0.7:0.7:0.7.

25. The composition of claim 18, wherein said ratio consists of 1:2:1:2:0.5:0.5:0.5.

26. A pharmaceutical composition comprising the composition of claim 18 and a pharmaceutically acceptable carrier.

27. A method for increasing a ratio of a number of cardiomyocytes to a number of non-cardiomyocytes within a population of cells comprising contacting said population of cells with the composition of claim 18.

28. The method of claim 27, wherein said non-cardiomyocytes comprise cardiac fibroblasts.

29. A method for treating cardiac injury comprising administering to a patient in need of such treatment a therapeutically effective amount of a composition of claim 18.

30. The method of claim 29, wherein said cardiac injury comprises myocardial infarction.

31. The method of claim 29, wherein a cause of said cardiac injury comprises reperfusion injury.

32. A method for stimulating vascular regeneration following ischemic damage comprising contacting tissue damaged by ischemic damage with the composition of claim 18.

33. A method for treating stroke comprising administering to a patient in need of said treatment a therapeutically effective amount of a composition of claim 18.

34. A method for enhancing wound healing comprising administering to a patient in need of such enhancing a therapeutically effective amount of a composition of claim 18.

35. The composition of claim 1, wherein when said composition comprises a ratio of G:M:T:H:dnT:dnW, M is present in an amount that is higher than other modRNA present in said composition.

36. The composition of claim 1, wherein when said composition comprises a ratio of G:M:T:H:dnT:dnW, H is present in an amount that is higher than other modRNA present in said composition.

37. The composition of claim 18, wherein when said composition comprises a ratio of G:M:T:H:A:dnT:dnW, M is present in an amount that is higher than other modRNA present in said composition.

38. The composition of claim 18, wherein when said composition comprises a ratio of G:M:T:H:A:dnT:dnW, H is present in an amount that is higher than other modRNA present in said composition.

39. A method for stimulating skeletal muscle regeneration comprising administering to a patient in need of said stimulation a therapeutically effective amount of the composition of claim 1.

40. A method for stimulating skeletal muscle regeneration comprising administering to a patient in need of said stimulation a therapeutically effective amount of the composition of claim 18.


SEQUENCE LISTING (92 sequences)

SEQ ID NO: 1 - DNA, 1329 bp, Artificial Sequence
  Mouse GATA4
atgtaccaaagcctggccatggccgccaaccacggccccccgcccggcgcctacgaagcaggtggccctggcgccttcatgcacagcgcgggcgccgcgtcctcgcccgtctacgtgcccactccgcgggtgccgtcctctgtgctgggcctgtcctacctgcagggcggtggcagtgccgctgcagctggaaccacctcgggtggcagctccggggccggcccgtcgggtgcagggcctgggacccagcagggtagccctggctggagccaagctggagccgagggagccgcctacaccccgccgcccgtgtccccgcgcttctctttcccggggactactgggtccctggcggccgctgccgccgctgccgcagcccgggaagctgcagcctacggcagtggcggcggggcggcgggcgctggtctggctggccgagagcagtacgggcgtccgggcttcgccggctcctactccagcccctacccagcctacatggccgacgtgggagcatcctgggccgcagccgctgccgcctctgccggccccttcgacagcccagtcctgcacagcctgcctggacgggccaaccctggaagacaccccaatctcgtagatatgtttgatgacttctcagaaggcagagagtgtgtcaattgtggggccatgtccaccccactctggaggcgagatgggacgggacactacctgtgcaatgcctgtggcctctatcacaagatgaacggcatcaaccggcccctcattaagcctcagcgccgcctgtccgcttcccgccgggtaggcctctcctgtgccaactgccagactaccaccaccacgctgtggcgtcgtaatgccgagggtgagcctgtatgtaatgcctgcggcctctacatgaagctccatggggttcccaggcctcttgcaatgcggaaggaggggattcaaaccagaaaacggaagcccaagaacctgaataaatctaagacgccagcaggtcctgctggtgagaccctccctccctccagtggtgcctccagcggtaactccagcaatgccactagcagcagcagcagcagtgaagagatgcgccccatcaagacagagcccgggctgtcatctcactatgggcacagcagctccatgtcccagacattcagtactgtgtccggccacgggccctccatccatccagtgctgtctgctctgaagctgtccccacaaggctatgcatctcctgtcactcagacatcgcaggccagctccaagcaggactcttggaacagcctggtcctggctgacagtcatggggacataatcaccgcgtaa

SEQ ID NO: 2 - DNA, 1401 bp, Artificial Sequence
  Mouse Mef2c
atggggagaaaaaagattcagattacgaggataatggatgagcgtaacagacaggtgacttttacgaagaggaaatttggattgatgaagaaggcttatgagctgagcgtgctgtgcgactgtgagattgcactgatcatcttcaacagcaccaacaagctgttccagtacgccagcactgacatggataaggtgttgctcaagtacaccgagtacaacgagccgcacgagagccggacaaactcagacattgtggagacattgagaaagaagggcctcaatggctgtgacagcccagatcccgatgcagacgattcagtaggtcacagccctgagtctgaggacaagtacaggaaaattaacgaagatattgatctaatgatcagcaggcaaagattgtgtgctgttccacctcccagctttgagatgccagttaccatcccagtgtccagccataacagtttggtgtacagcaatcctgtcagcacactgggaaaccccaatcttctgccactggcccacccgtctctgcagaggaatagtatgtctcctggtgtaacacatagacctccaagtgcaggtaacacaggcggtctgatgggcggagatctgacatccggtgcaggcaccagcgcagggaatggatacggcaacccccggaactcaccaggcctgctggtctcacctggtaacctgaacaagaatatacaagccaaatctcctccccctatgaatctaggaatgaataatcgtaagccagatctccgcgttcttatcccacctggcagcaagaacacgatgccatcagtgaatcaaaggataaataactcccagtcggctcagtcattggctaccccggtggtttccgtagcaactcctactttaccaggacaaggaatgggaggatatccatcagccatttcaacaacatatggtactgagtactctctgagtagcgcagatctgtcatctctgtctggcttcaacactgccagtgcgctccacctcggctctgtaactggctggcagcagcagcacctacataacatgccgccatctgccctcagtcagttgggagcttgcactagcactcatttatctcagagttcaaatctctccctgccttctactcaaagcctcagcatcaagtcagaacctgtttctcctcctagagaccgtaccaccaccccttcgagatacccacaacacaccacgcgccacgaggcggggaggtctcctgttgacagcttgagcagctgtagcagttcctacgatgggagcgaccgagaggatcaccggaacgaattccactcccccattggactcaccagaccttcgccggacgaaagggaaagtccttcagtcaagcgcatgcgactctctgaaggatgggcaacatga

SEQ ID NO: 3 - DNA, 654 bp, Artificial Sequence
  Mouse Tbx5
atgagtctggtggggggctttccccaccaccccgtggtgcaccatgagggctacccattcgccgcagccgccgccgccgctgccgccgccgccgccagccgctgcagccacgaggagaacccctacttccacggctggcttattggccacccggagatgtcgccccccgactacagtatggccctgtcctacagccccgagtacgccagcggtgccgcgggcctggaccactcccattatgggggagtgccgcccggcgccgggcctcccggcctgggggggccgcgcccggtgaagcgccggggcaccgccaaccgcaaggagcggcgcaggactcagagcatcaacagcgccttcgccgagctgcgcgagtgcatccccaacgtgcccgccgacaccaaactctccaagatcaagacactgcgcctggccaccagctacatcgcctacctcatggatctgctggccaaggacgaccagaacggagaggcggaggccttcaaggcggagatcaagaagaccgacgtgaaagaggagaagaggaagaaagagctgaatgagatcttgaaaagcacagtgagcagcaacgacaagaaaaccaaaggccggacaggctggccacagcacgtctgggccctggagctcaagcagtga

SEQ ID NO: 4 - DNA, 654 bp, Artificial Sequence; identical to SEQ ID NO: 3
  Mouse Hand2

SEQ ID NO: 5 - DNA, 1413 bp, Artificial Sequence
  Mouse Gata4-Flag
atgtaccaaagcctggccatggccgccaaccacggccccccgcccggcgcctacgaagcaggtggccctggcgccttcatgcacagcgcgggcgccgcgtcctcgcccgtctacgtgcccactccgcgggtgccgtcctctgtgctgggcctgtcctacctgcagggcggtggcagtgccgctgcagctggaaccacctcgggtggcagctccggggccggcccgtcgggtgcagggcctgggacccagcagggtagccctggctggagccaagctggagccgagggagccgcctacaccccgccgcccgtgtccccgcgcttctctttcccggggactactgggtccctggcggccgctgccgccgctgccgcagcccgggaagctgcagcctacggcagtggcggcggggcggcgggcgctggtctggctggccgagagcagtacgggcgtccgggcttcgccggctcctactccagcccctacccagcctacatggccgacgtgggagcatcctgggccgcagccgctgccgcctctgccggccccttcgacagcccagtcctgcacagcctgcctggacgggccaaccctggaagacaccccaatctcgatatgtttgatgacttctcagaaggcagagagtgtgtcaattgtggggccatgtccaccccactctggaggcgagatgggacgggacactacctgtgcaatgcctgtggcctctatcacaagatgaacggcatcaaccggcccctcattaagcctcagcgccgcctgtccgcttcccgccgggtaggcctctcctgtgccaactgccagactaccaccaccacgctgtggcgtcgtaatgccgagggtgagcctgtatgtaatgcctgcggcctctacatgaagctccatggggttcccaggcctcttgcaatgcggaaggaggggattcaaaccagaaaacggaagcccaagaacctgaataaatctaagacgccagcaggtcctgctggtgagaccctccctccctccagtggtgcctccagcggtaactccagcaatgccactagcagcagcagcagcagtgaagagatgcgccccatcaagacagagcccgggctgtcatctcactatgggcacagcagctccatgtcccagacattcagtactgtgtccggccacgggccctccatccatccagtgctgtctgctctgaagctgtccccacaaggctatgcatctcctgtcactcagacatcgcaggccagctccaagcaggactcttggaacagcctggtcctggctgacagtcatggggacataatcaccgcgggaggcggtggagccgactacaaggaccacgacggcgactacaaggaccacgacatcgactacaaggacgacgacgacaaggggcccgtt

SEQ ID NO: 6 - DNA, 1488 bp, Artificial Sequence
  Mouse Mef2c-Flag
atggggagaaaaaagattcagattacgaggataatggatgagcgtaacagacaggtgacttttacgaagaggaaatttggattgatgaagaaggcttatgagctgagcgtgctgtgcgactgtgagattgcactgatcatcttcaacagcaccaacaagctgttccagtacgccagcactgacatggataaggtgttgctcaagtacaccgagtacaacgagccgcacgagagccggacaaactcagacattgtggagacattgagaaagaagggcctcaatggctgtgacagcccagatcccgatgcagacgattcagtaggtcacagccctgagtctgaggacaagtacaggaaaattaacgaagatattgatctaatgatcagcaggcaaagattgtgtgctgttccacctcccagctttgagatgccagttaccatcccagtgtccagccataacagtttggtgtacagcaatcctgtcagcacactgggaaaccccaatcttctgccactggcccacccgtctctgcagaggaatagtatgtctcctggtgtaacacatagacctccaagtgcaggtaacacaggcggtctgatgggcggagatctgacatccggtgcaggcaccagcgcagggaatggatacggcaacccccggaactcaccaggcctgctggtctcacctggtaacctgaacaagaatatacaagccaaatctcctccccctatgaatctaggaatgaataatcgtaagccagatctccgcgttcttatcccacctggcagcaagaacacgatgccatcagtgaatcaaaggataaataactcccagtcggctcagtcattggctaccccggtggtttccgtagcaactcctactttaccaggacaaggaatgggaggatatccatcagccatttcaacaacatatggtactgagtactctctgagtagcgcagatctgtcatctctgtctggcttcaacactgccagtgcgctccacctcggctctgtaactggctggcagcagcagcacctacataacatgccgccatctgccctcagtcagttgggagcttgcactagcactcatttatctcagagttcaaatctctccctgccttctactcaaagcctcagcatcaagtcagaacctgtttctcctcctagagaccgtaccaccaccccttcgagatacccacaacacaccacgcgccacgaggcggggaggtctcctgttgacagcttgagcagctgtagcagttcctacgatgggagcgaccgagaggatcaccggaacgaattccactcccccattggactcaccagaccttcgccggacgaaagggaaagtccttcagtcaagcgcatgcgactctctgaaggatgggcaacaggaggcggtggagccgactacaaggaccacgacggcgactacaaggaccacgacatcgactacaaggacgacgacgacaaggggcccgtt

SEQ ID NO: 7 - DNA, 1644 bp, Artificial Sequence
  Mouse TBX5-Flag
atggccgatacagatgagggctttggcctggcgcgcacgcctctggagcctgattccaaagacaggtcttgcgattcgaaacctgagagtgctctgggggctcccagcaagtctccatcatccccgcaggctgccttcacccagcagggcatggaaggaatcaaggtgtttcttcatgaacgtgaactgtggctgaagttccacgaagtgggcacagagatgatcatcaccaaggcagggaggagaatgtttcctagttacaaagtgaaggtgactggccttaatcccaaaacgaagtatattcttctcatggatattgttcccgcagacgaccacagatataaatttgctgataacaaatggtccgtaactggcaaagcagagcctgccatgccggggcgcctttacgtgcacccggactccccagcaaccggagcccactggatgcgacaacttgtctccttccagaagctcaaactcaccaacaaccacctggacccgtttggacacattatcctgaactccatgcacaaataccagccccgattacacatcgtgaaagcagacgaaaataatgggttcggttcaaagaacactgcgttttgcacccacgtcttcccggagacagcttttatcgctgtgacttcgtaccagaatcacaagatcacacagctgaaaattgagaacaaccccttcgccaaaggctttcggggcagtgatgacctggagttacacaggatgtctcggatgcaaagtaaagagtatcctgtggttcccaggagcacagtgaggcacaaagtcacctccaaccacagccccttcagcagcgagacccgagctctctccacctcatccaatttagggtcccagtaccagtgtgagaatggtgtctctggcccctcccaggaccttctgcccccacctaacccatacccactggcccaggagcacagccaaatttaccactgtaccaagaggaaagatgaggaatgttccagcacggagcacccctataagaagccgtacatggagacatcccccagcgaggaagacaccttctatcgctcgggctacccccagcagcagggcctgagtacctcttacaggacagagtcggcccagcggcaggcctgcatgtatgccagctccgctccccccagcgagcccgtgcctagcctggaggacatcagctgtaacacatggcccagcatgccctcctatagcagctgtaccgtcaccaccgtgcagcccatggaccgtcttccctaccagcacttctccgctcatttcacctcggggcccctggtccctcggttggctggcatggccaaccatggttctccccagctcggcgaagggatgtttcagcaccagacctcagtggcccatcagcctgtggtcaggcagtgcgggcctcagactggccttcagtctccgggcggcctccagcccccagagtttctctacactcacggcgtgcccaggaccctgtccccccatcagtatcactcggtacacggcgtcggcatggtgccagagtggagtgagaatagcggaggcggtggagccgactacaaggaccacgacggcgactacaaggaccacgacatcgactacaaggacgacgacgacaaggggcccgtt

SEQ ID NO: 8 - DNA, 1188 bp, Artificial Sequence
  ASAH1
atgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 9 - DNA, 1332 bp, Artificial Sequence
  Human GATA4
atgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgtagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaa

SEQ ID NO: 10 - DNA, 1050 bp, Artificial Sequence
  Human Mef2c
atggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggaggcattgaacaagaaagaaaacaaaggctgtgaaagccccgatcccgactcctcttatgcactcaccccacgcactgaagaaaaatacaaaaaaattaatgaagaatttgataatatgatcaagagtcataaaattcctgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccacca

SEQ ID NO: 11 - DNA, 1557 bp, Artificial Sequence
  Human Tbx5
atggccgacgcagacgagggctttggcctggcgcacacgcctctggagcctgacgcaaaagacctgccctgcgattcgaaacccgagagcgcgctcggggcccccagcaagtccccgtcgtccccgcaggccgccttcacccagcagggcatggagggaatcaaagtgtttctccatgaaagagaactgtggctaaaattccacgaagtgggcacggaaatgatcataaccaaggctggaaggcggatgtttcccagttacaaagtgaaggtgacgggccttaatcccaaaacgaagtacattcttctcatggacattgtacctgccgacgatcacagatacaaattcgcagataataaatggtctgtgacgggcaaagctgagcccgccatgcctggccgcctgtacgtgcacccagactcccccgccaccggggcgcattggatgaggcagctcgtctccttccagaaactcaagctcaccaacaaccacctggacccatttgggcatattattctaaattccatgcacaaataccagcctagattacacatcgtgaaagcggatgaaaataatggatttggctcaaaaaatacagcgttctgcactcacgtctttcctgagactgcgtttatagcagtgacttcctaccagaaccacaagatcacgcaattaaagattgagaataatccctttgccaaaggatttcggggcagtgatgacatggagctgcacagaatgtcaagaatgcaaagtaaagaatatcccgtggtccccaggagcaccgtgaggcaaaaagtggcctccaaccacagtcctttcagcagcgagtctcgagctctctccacctcatccaatttggggtcccaataccagtgtgagaatggtgtttccggcccctcccaggacctcctgcctccacccaacccatacccactgccccaggagcatagccaaatttaccattgtaccaagaggaaagaggaagaatgttccaccacagaccatccctataagaagccctacatggagacatcacccagtgaagaagattccttctaccgctctagctatccacagcagcagggcctgggtgcctcctacaggacagagtcggcacagcggcaagcttgcatgtatgccagctctgcgccccccagcgagcctgtgcccagcctagaggacatcagctgcaacacgtggccaagcatgccttcctacagcagctgcaccgtcaccaccgtgcagcccatggacaggctaccctaccagcacttctccgctcacttcacctcggggcccctggtccctcggctggctggcatggccaaccatggctccccacagctgggagagggaatgttccagcaccagacctccgtggcccaccagcctgtggtcaggcagtgtgggcctcagactggcctgcagtcccctggcacccttcagccccctgagttcctctactctcatggcgtgccaaggactctatcccctcatcagtaccactctgtgcacggagttggcatggtgccagagtggagcgacaatagctaa

SEQ ID NO: 12 - DNA, 654 bp, Artificial Sequence
  Human Hand2
atgagtctggtaggtggttttccccaccacccggtggtgcaccacgagggctacccgtttgccgccgccgccgccgcagctgccgccgccgccgccagccgctgcagccatgaggagaacccctacttccatggctggctcatcggccaccccgagatgtcgccccccgactacagcatggccctgtcctacagccccgagtatgccagcggcgccgccggcctggaccactcccattacgggggggtgccgccgggcgccgggcccccgggcctgggggggccgcgcccggtgaagcgccgaggcaccgccaaccgcaaggagcggcgcaggactcagagcatcaacagcgccttcgccgaactgcgcgagtgcatccccaacgtacccgccgacaccaaactctccaaaatcaagaccctgcgcctggccaccagctacatcgcctacctcatggacctgctggccaaggacgaccagaatggcgaggcggaggccttcaaggcagagatcaagaagaccgacgtgaaagaggagaagaggaagaaggagctgaacgaaatcttgaaaagcacagtgagcagcaacgacaagaaaaccaaaggccggacgggctggccgcagcacgtctgggccctggagctcaagcagtga

SEQ ID NO: 13 - DNA, 1422 bp, Artificial Sequence
  Human GATA4-Flag
atgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgtagatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtccccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaaggaggcggtggagccgactacaaggaccacgacggcgactacaaggaccacgacatcgactacaaggacgacgacgacaaggggcccgtt

SEQ ID NO: 14 - DNA, 1140 bp, Artificial Sequence
  Human Mef2c-Flag
atggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggaggcattgaacaagaaagaaaacaaaggctgtgaaagccccgatcccgactcctcttatgcactcaccccacgcactgaagaaaaatacaaaaaaattaatgaagaatttgataatatgatcaagagtcataaaattcctgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccaggaggcggtggagccgactacaaggaccacgacggcgactacaaggaccacgacatcgactacaaggacgacgacgacaaggggcccgtt

SEQ ID NO: 15 - DNA, 1647 bp, Artificial Sequence
  Human Tbx5- Flag
atggccgacgcagacgagggctttggcctggcgcacacgcctctggagcctgacgcaaaagacctgccctgcgattcgaaacccgagagcgcgctcggggcccccagcaagtccccgtcgtccccgcaggccgccttcacccagcagggcatggagggaatcaaagtgtttctccatgaaagagaactgtggctaaaattccacgaagtgggcacggaaatgatcataaccaaggctggaaggcggatgtttcccagttacaaagtgaaggtgacgggccttaatcccaaaacgaagtacattcttctcatggacattgtacctgccgacgatcacagatacaaattcgcagataataaatggtctgtgacgggcaaagctgagcccgccatgcctggccgcctgtacgtgcacccagactcccccgccaccggggcgcattggatgaggcagctcgtctccttccagaaactcaagctcaccaacaaccacctggacccatttgggcatattattctaaattccatgcacaaataccagcctagattacacatcgtgaaagcggatgaaaataatggatttggctcaaaaaatacagcgttctgcactcacgtctttcctgagactgcgtttatagcagtgacttcctaccagaaccacaagatcacgcaattaaagattgagaataatccctttgccaaaggatttcggggcagtgatgacatggagctgcacagaatgtcaagaatgcaaagtaaagaatatcccgtggtccccaggagcaccgtgaggcaaaaagtggcctccaaccacagtcctttcagcagcgagtctcgagctctctccacctcatccaatttggggtcccaataccagtgtgagaatggtgtttccggcccctcccaggacctcctgcctccacccaacccatacccactgccccaggagcatagccaaatttaccattgtaccaagaggaaagaggaagaatgttccaccacagaccatccctataagaagccctacatggagacatcacccagtgaagaagattccttctaccgctctagctatccacagcagcagggcctgggtgcctcctacaggacagagtcggcacagcggcaagcttgcatgtatgccagctctgcgccccccagcgagcctgtgcccagcctagaggacatcagctgcaacacgtggccaagcatgccttcctacagcagctgcaccgtcaccaccgtgcagcccatggacaggctaccctaccagcacttctccgctcacttcacctcggggcccctggtccctcggctggctggcatggccaaccatggctccccacagctgggagagggaatgttccagcaccagacctccgtggcccaccagcctgtggtcaggcagtgtgggcctcagactggcctgcagtcccctggcacccttcagccccctgagttcctctactctcatggcgtgccaaggactctatcccctcatcagtaccactctgtgcacggagttggcatggtgccagagtggagcgacaatagctaaggaggcggtggagccgactacaaggaccacgacggcgactacaaggaccacgacatcgactacaaggacgacgacgacaaggggcccgtt

SEQ ID NO: 16 - DNA, 1188 bp, Artificial Sequence; identical to SEQ ID NO: 8
  ACVI

SEQ ID NO: 17 - DNA, 609 bp, Artificial Sequence
  dnTGFbeta
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttggatccatggactacaaagacgatgacgataaatag

SEQ ID NO: 18 - DNA, 657 bp, Artificial Sequence
  CCN5
atgagaggcacaccgaagacccacctcctggccttctccctcctctgcctcctctcaaaggtgcgtacccagctgtgcccgacaccatgtacctgcccctggccacctccccgatgcccgctgggagtacccctggtgctggatggctgtggctgctgccgggtatgtgcacggcggctgggggagccctgcgaccaactccacgtctgcgacgccagccagggcctggtctgccagcccggggcaggacccggtggccggggggccctgtgcctctgtaagcaggaccccagttttctggccttgtctcttccctgccccctggtgtcccctgcccagaatggagcacggcctggggaccctgctcgaccacctgtgggctgggcatggccacccgggtgtccaaccagaaccgcttctgccgactggagacccagcgccgcctgtgcctgtccaggccctgcccaccctccaggggtcgcagtccacaaaacagtgccttctagagccgggctgggaatggggacacggtgtccaccatccccagctggtggccctgtgcctgggccctgggctgatggaagatggtccgtgcccaggcccttggctgcaggcaacactttagcttgggtccaccatgcagaacaccaatattaa

SEQ ID NO: 19 - DNA, 678 bp, Artificial Sequence
  dnWnt8a
  misc_feature (669)..(669): n is a, c, g, or t
  misc_feature (672)..(672): n is a, c, g, or t
atgcaaaacaccactttgttcatccttgcaactcttctgatcttctgcccattcttcactgcctcagcatggtcagtcaataactttctgatgacaggacccaaggcatatctgacatactcagcgagtgttgccgtgggtgcgcagaatggaattgaggagtgtaaatatcagtttgcgtgggaaagatggaattgccctgaaagtaccctgcagcttgctacccacaatggacttcgaagtgcaaccagagaaacctcctttgtgcatgccattagctcagcgggagttatgtatacactgacgagaaactgcagcatgggggactttgataactgtggatgtgatgactccagaaatggccgcatcggtggccgaggctgggtatggggcggctgcagtgataatgcagaatttggtgagcggatctctaaactattcgtcgatggcttggagacgggacaagatgccagagccctaatgaacttgcataacaatgaagcaggaagacttgcagtgaaagagacaatgaaaaggacttgcaagtgccatggaatatctggaagttgcagcatacaaacttgctggcttcagctggccgagtttcgggatattggcaatcccttaaagatcaagcacgaccaagcgctaaagcttgagatggacaanagnaaatga

SEQ ID NO: 20 - DNA, 1083 bp, Artificial Sequence
  WNT5a
atgtcttccaagttcttcctaatggctttggccacgtttttctccttcgcccaggttgttatagaagctaattcttggtggtctctaggtatgaataaccctgttcagatgtcagaagtatatatcataggtgcacagcctctctgcagccaactggcaggactttctcaaggacagaagaaactctgccacttgtatcaggaccacatgcagtacattggagaaggtgcgaagacaggcatcaaggaatgccagtaccagttccggcatcggagatggaactgcagcacagtggacaatacttctgtctttggcagggtgatgcaaataggcagccgagagacggccttcacgtacgcggtgagcgcagctggggtggtgaacgccatgagccgagcatgccgggagggcgagctgtctacctgtggctgcagccgcgctgcgcgccccaaggacctgcctcgggactggttgtggggcggctgcggagacaacatcgactatggctaccgcttcgccaaggagttcgtggacgctagagaaagggaacgaatccacgctaagggttcctatgagagcgcacgcatcctcatgaacttacacaacaatgaagcaggccgtaggacagtatacaacctggcagatgtagcctgtaagtgtcatggagtgtctggctcctgtagcctcaagacgtgctggctgcagctggcggacttccggaaggtgggcgatgccctcaaggagaagtatgatagcgcggcggccatgaggctcaacagccggggcaagctggtgcaggtcaacagccgcttcaactccccgaccacgcaggacctggtctacatcgaccccagtccggactactgtgtgcgcaacgagagcactggctcgctgggcacgcagggacgcctgtgcaacaagacctcagaggggatggacggctgcgagctcatgtgctgtgggcgtggctatgaccagtttaagacagtgcagaccgaacgctgtcattgcaagtttcactggtgctgctatgtcaaatgcaagaagtgcacggagattgtggatcagttcgtgtgcaaatag

SEQ ID NO: 21 - DNA, 915 bp, Artificial Sequence
  SV40 Large T
atgcctaggcttttgcaaaaagctttgcaaatatggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgaggtatttgcttcttccttaaatcctggtgttgatgcaatgtactgcaaacaatggcctgagtgtgcaaagaaaatgtctactaactgcatatgcttgctgtgcttactgaggatgaagcatgaaaatagaaaattatacaggaaagatccacttgtgtgggttgattgctactgcttcgattgctttagaatgtggtttggacttgatctttgtgaaggaaccttacttctgtggtgtgacataattggacaaactacctacagagatttaaagctataaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaataa

SEQ ID NO: 22 - DNA, 915 bp, Artificial Sequence
  SRF
atgatcaccagtgagaccggcaaggcactgattcagacctgcctcaactcgccagactctccaccccgttcagaccccacaacagaccagagaatgagtgccactggctttgaagagacagatctcacctaccaggtgtcggagtctgacagcagtggggagaccaaggacacactgaagccggcgttcacagtcaccaacctgccgggtacaacctccaccatccaaacagcacctagcacctctaccaccatgcaagtcagcagcggcccctcctttcccatcaccaactacctggcaccagtgtctgctagtgtcagccccagtgctgtcagcagtgccaatgggactgtgctgaagagtacaggcagcggccctgtctcctctgggggccttatgcagctgcctaccagcttcaccctcatgcctggtggggcagtggcccagcaggtcccagtgcaggccattcaagtgcaccaggccccacagcaagcgtctccctcccgtgacagcagcacagacctcacgcagacctcctccagcgggacagtgacgctgcccgccaccatcatgacgtcatccgtgcccacaactgtgggtggccacatgatgtaccctagcccgcatgcggtgatgtatgcccccacctcgggcctgggtgatggcagcctcaccgtgctgaatgccttctcccaggcaccatccaccatgcaggtgtcacacagccaggtccaggagccaggtggcgtcccccaggtgttcctgacagcatcatctgggacagtgcagatccctgtttcagcagttcagctccaccagatggctgtgatagggcagcaggccgggagcagcagcaacctcaccgagctacaggtggtgaacctggacaccgcccacagcaccaagagtgaatga

SEQ ID NO: 23 - DNA, 2961 bp, Artificial Sequence
  Human Myocardin
atgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccagggccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagaactcaggagcacacgatggccatcctccaagcttctctccccattcttccagcctccacccgcccttctctggagcccaagcagacagcagtcatggtgccgggggaaacccttgtcccaaaagcccatgtgtacagcaaaagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtggtag

SEQ ID NO: 24 - DNA, 1458 bp, Artificial Sequence
  ETS1
atgagctactttgtggattctgctgggagcagccccgtcccttactcagcgcctcgtcctgcagtggtgaggcaaggacctagcaacacttatgaagatcctcgaatgaactgtggtttccagtccaattatcaccagcaaagaccttgctaccccttttgggatgagatggcaactcaggaagttcctactggtcttgaacactgtgtctcagatatggaatgtgcagatgtcccactattaactccaagcagcaaagaaatgatgtctcaagcattaaaagctactttcagtggtttcactaaagaacagcaacgactggggatcccaaaagacccccggcagtggacagaaacccatgttcgggactgggtgatgtgggctgtgaatgaattcagcctgaaaggtgtagacttccagaagttctgtatgaatggagcagccctctgcgccctgggtaaagactgctttctcgagctggccccagactttgttggggacatcttatgggaacatctagagatcctgcagaaagaggatgtgaaaccatatcaagttaatggagtcaacccagcctatccagaatcccgctatacctcggattacttcattagctatggtattgagcatgcccagtgtgttccaccatcggagttctcagagcccagcttcatcacagagtcctatcagacgctccatcccatcagctcggaagagctcctctccctcaagtatgagaatgactacccctcggtcattctccgagaccctctccagacagacaccttgcagaatgactactttgctatcaaacaagaagtcgtcaccccagacaacatgtgcatggggaggaccagtcgtggtaaactcgggggccaggactcttttgaaagcatagagagctacgatagttgtgatcgcctcacccagtcctggagcagccagtcatctttcaacagcctgcagcgtgttccctcctatgacagcttcgactcagaggactatccggctgccctgcccaaccacaagcccaagggcaccttcaaggactatgtgcgggaccgtgctgacctcaataaggacaagcctgtcattcctgctgctgccctagctggctacacaggcagtggaccaatccagctatggcagtttcttctggaattactcactgataaatcctgtcagtcttttatcagctggacaggagatggctgggaattcaaactttctgacccagatgaggtggccaggagatggggaaagaggaaaaacaaacctaagatgaattatgagaaactgagccgtggcctacgctactattacgacaaaaacatcatccacaagacagcggggaaacgctacgtgtaccgctttgtgtgtgacctgcagagcctgctggggtacacccctgaggagctgcacgccatgctggacgtcaagccagatgccgacgagtga

SEQ ID NO: 25 - DNA, 1830 bp, Artificial Sequence
  ETS2
atggggtcggctcaatttcagggccttattacccaagcccggctgcccttcggtgccaccagcaccactgctccgtcgctgcggaattccaaaggcaggtttggcgttagggccttggccccagagaggacgccgagcgctccacggaaagtctccgcccggctcccagggcgcacactcgcgcgcacgtggggccgaggccctgctcccggggcctcagggccagccggcgagggacccagccgagtgacagcaggaggcggagggaagctcagagctcccggagccgcccggccagcgtccggcctccctgatcgtctctggccggcgccctcgccctcgcccggcgcgcaccgagcagccgcgggcgccgagcagccaccgtcccgaccaagcgccggccctgcccgcagcggcaggatgaatgatttcggaatcaagaatatggaccaggtagcccctgtggctaacagttacagagggacactcaagcgccagccagcctttgacacctttgatgggtccctgtttgctgtttttccttctctaaatgaagagcaaacactgcaagaagtgccaacaggcttggattccatttctcatgactccgccaactgtgaattgcctttgttaaccccgtgcagcaaggctgtgatgagtcaagccttaaaagctaccttcagtggcttcaaaaaggaacagcggcgcctgggcattccaaagaacccctggctgtggagtgagcaacaggtatgccagtggcttctctgggccaccaatgagttcagtctggtgaacgtgaatctgcagaggttcggcatgaatggccagatgctgtgtaaccttggcaaggaacgctttctggagctggcacctgactttgtgggtgacattctctgggaacatctggagcaaatgatcaaagaaaaccaagaaaagacagaagatcaatatgaagaaaattcacacctcacctccgttcctcattggattaacagcaatacattaggttttggcacagagcaggcgccctatggaatgcagacacagaattaccccaaaggcggcctcctggacagcatgtgtccggcctccacacccagcgtactcagctctgagcaggagtttcagatgttccccaagtctcggctcagctccgtcagcgtcacctactgctctgtcagtcaggacttcccaggcagcaacttgaatttgctcaccaacaattctgggactcccaaagaccacgactcccctgagaacggtgcggacagcttcgagagctcagactccctcctccagtcctggaacagccagtcgtccttgctggatgtgcaacgggttccttccttcgagagcttcgaagatgactgcagccagtctctctgcctcaataagccaaccatgtctttcaaggattacatccaagagaggagtgacccagtggagcaaggcaaaccagttatacctgcagctgtgctggccggcttcacaggaagtggacctattcagctgtggcagtttctcctggagctgctatcagacaaatcctgccagtcattcatcagctggactggagacggatgggagtttaagctcgccgaccccgatgaggtggcccgccggtggggaaagaggaaaaataagcccaagatgaactacgagaagctgagccggggcttacgctactattacgacaagaacatcatccacaagacgtcggggaagcgctacgtgtaccgcttcgtgtgcgacctccagaacttgctggggttcacgcccgaggaactgcacgccatcctgggcgtccagcccgacacggaggactga

SEQ ID NO: 26 - DNA, 1308 bp, Artificial Sequence
  ESRRG
atgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccatataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcgtttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtctga

SEQ ID NO: 27 - DNA, 1452 bp, Artificial Sequence
  SMARCD3
atggccgcggacgaagttgccggaggggcgcgcaaagccacgaaaagcaaactttttgagtttctggtccatggggtgcgccccgggatgccgtctggagcccggatgccccaccagggggcgcccatgggccccccgggctccccgtacatgggcagccccgccgtgcgacccggcctggcccccgcgggcatggagcccgcccgcaagcgagcagcgcccccgcccgggcagagccaggcacagagccagggccagccggtgcccaccgcccccgcgcggagccgcagtgccaagaggaggaagatggctgacaaaatcctccctcaaaggattcgggagctggtccccgagtcccaggcttacatggacctcttggcatttgagaggaaactggatcaaaccatcatgcggaagcgggtggacatccaggaggctctgaagaggcccatgaagcaaaagcggaagctgcgactctatatctccaacacttttaaccctgcgaagcctgatgctgaggattccgacggcagcattgcctcctgggagctacgggtggaggggaagctcctggatgatcccagcaaacagaagcggaagttctcttctttcttcaagagtttggtcatcgagctggacaaagatctttatggccctgacaaccacctcgttgagtggcatcggacacccacgacccaggagacggacggcttccaggtgaaacggcctggggacctgagtgtgcgctgcacgctgctcctcatgctggactaccagcctccccagttcaaactggatccccgcctagcccggctgctggggctgcacacacagagccgctcagccattgtccaggccctgtggcagtatgtgaagaccaacaggctgcaggactcccatgacaaggaatacatcaatggggacaagtatttccagcagatttttgattgtccccggctgaagttttctgagattccccagcgcctcacagccctgctattgccccctgacccaattgtcatcaaccatgtcatcagcgtggacccttcagaccagaagaagacggcgtgctatgacattgacgtggaggtggaggagccattaaaggggcagatgagcagcttcctcctatccacggccaaccagcaggagatcagtgctctggacagtaagatccatgagacgattgagtccataaaccagctcaagatccagagggacttcatgctaagcttctccagagaccccaaaggctatgtccaagacctgctccgctcccagagccgggacctcaaggtgatgacagatgtagccggcaaccctgaagaggagcgccgggctgagttctaccaccagccctggtcccaggaggccgtcagtcgctacttctactgcaagatccagcagcgcaggcaggagctggagcagtcgctggttgtgcgcaacacctag

SEQ ID NO: 28 - DNA, 3297 bp, Artificial Sequence
  ZFPM2
atgtcccggcgaaagcaaagcaaaccccggcagatcaaacgtgatgatgaaggaatccaggagacagcagaatcagatggggacacacagtcagagaaaccggggcaacctggagttgagacagacgactgggatggaccaggagagctggaggtgtttcagaaagatggggaacgaaaaattcagagtcgacagcaacttccagtgggaacaacctgggggccgtttcctgggaagatggacttgaataataattctttgaagacaaaggctcaggtcccaatggtgctgactgctggtcccaagtggttgctggatgtgacttggcaaggagtggaagacaacaaaaacaactgcattgtgtacagcaaagggggtcagctttggtgtacaactacgaaggccatctctgagggtgaagagctaattgcctttgtggtggattttgactcaaggctacaagctgccagtcagatgactctcacagaagggatgtaccctgcacgcctgctggactcaattcagctgcttcctcagcaagctgccatggcttctattttgcccacagctattgtcaataaggatatattcccttgcaagtcctgtggcatctggtatcggagtgagcggaatctgcaggcccatttgatgtactactgcagtgggaggcaaagagaagctgctccggtgtcagaggaaaatgaagacagtgcccatcagatttccagcctgtgccccttcccacagtgcaccaagagcttttcaaatgctcgagctctagaaatgcacctgaattcacacagtggagtgaaaatggaagaattcctgccccctggtgctagtctaaaatgcaccgtctgtagctacactgctgattccgtgatcaactttcaccaacacctgttctcccatctcactcaagctgccttccgatgtaatcactgccatttcggcttccagactcagagggagttattgcagcaccaggagctccatgtccctagcggcaaacttcccagagaaagtgacatggaacactctccaagtgcaactgaagacagcttacagccagccacagacttattgaccagaagcgaacttccccagagccaaaaggccatgcagactaaagatgcgagctctgacacagagctggacaagtgtgagaaaaagactcagctctttctcacgaaccagagaccagagatacagcctacaacaaataaacaaagcttttcttacacaaaaataaagtctgagccctctagcccaagacttgcctcatctccagttcagcctaatattgggccttctttccctgtgggccctttcctatctcagttttctttcccccaagatatcaccatggtccctcaagcttcagagatcttagctaagatgtctgaactggtgcatcggcgactgaggcatggcagtagtagctaccctcccgtcatttacagccctttgatgcccaagggggctacttgttttgagtgtaacataacattcaataatttggataattatctagtgcacaaaaagcattattgcagcagccgatggcagcagatggctaagtccccagagttccctagtgtgtcagaaaagatgcctgaagctttgagtcccaacactggccaaacctccataaaccttctcaacccagctgctcattctgctgatcctgagaatccacttcttcaaacatcttgcatcaattcttccactgtcttagatttaattgggccaaatgggaagggccatgacaaggacttttccactcaaactaagaagctctccacctccagtaacaatgatgacaaaattaatggaaaacctgttgatgtgaaaaatcccagtgtccccttagtggatggggaaagtgacccaaataagactacctgtgaagcttgcaacattaccttcagccggcacgaaacatacatggtccacaaacagtattactgtgctacacgccacgaccctccactgaagaggtctgcttccaacaaagtgcctgccatgcagagaaccatgcgcacacgcaagcgcagaaagatgtatgagatgtgcctacctgagcaggaacaaaggcctccactggttcagcagagatttcttgacgtagccaacctcaataatccttgtacctccactcaagaacccacagaagggctaggagagtgctaccacccaagatgtgatatctttccaggaattgtctctaaacacttggaaacttctctgacgatcaacaagtgtgttccagtttccaaatgtgatactactcattccagtgtttcctgcctagagatggacgtgcccatagatctcagcaaaaagtgtttatctcagtctgagcggacgaccacgtctcccaaaaggctgctggactatcacgagtgcactgtgtgcaagatcagtttcaataaggtagaaaactatctggcccacaagcagaatttctgcccggttactgcacatcagcgtaatgacctgggtcaactggacggcaaagtgtttccgaatccagaaagcgaacgaaacagccctgatgtcagctacgaaagaagcataataaaatgtgagaaaaatgggaatttgaagcagccttcccccaatggaaacttattttcatcccacctagcaaccctgcaaggcttgaaggtctttagtgaagctgctcagctcattgctacaaaagaagaaaacagacatttgtttcttccacaatgcctttaccctggagcaataaagaaagcaaaaggagccgaccagctttctccatattatggaatcaagccaagtgattatatttctggttctcttgtcatccataacactgacatcgagcaaagcagaaatgcagaaaatgaatctcctaaaggccaggcttcctcaaatgggtgtgctgcgctgaagaaagattctctgccattgttgcccaaaaatcgaggaatggtaatagtgaatggtggactgaaacaagatgagagacctgctgccaacccacagcaagagaacatttcccagaatcctcagcacgaagacgaccacaaatctccctcgtggatctctgagaacccattagctgccaatgagaatgtctcaccaggaattccctcagcagaggaacagttgtctagtatagcaaaaggtgtgaatggttccagccaggctccaaccagtgggaaatattgccggctatgtgatatccagttcaacaacctttcaaactttataactcacaagaagttttattgctcatcacatgcagcagaacatgtcaaatga

SEQ ID NO: 29 - DNA, 1033 bp, Artificial Sequence
  DN SNAI1
cactatagggcgaattggcggaaggccgtcaaggccgcatttggaccctcgtacagaagctaatacgactcactatagggaaataagagagaaaataagagtaagaagaaatataagagccaccatgaggaagccgtccgacccccgccggaagcccaactatagcgagctgcaggacgcgtgtgtggagttcaccttccagcagccctacgaccaggcccacctgctggccgccatccctccgcccgaggtcctcaaccccgccgcttcgctgcccaccctcatctgggactctctcctggtaccccaagtgcggccggttgcctgggccaccctcccgctgcgggagagccccaaggccgtagagctgacctcgctgtccgatgaggacagtggcaaaagctcccagccgcccagcccgccctcgccggcgccgtcgtccttctcgtccacctcggcctcctccctggaggccgaggccttcatcgccttccctggcttgggccaacttcccaagcagctggccaggctctcggtggccaaggacccccagtcgcggaagatcttcaactgcaaatattgtaacaaggagtacctcagcctgggcgctctgaagatgcacatccgaagccacacgctgccttgtgtctgcacgacctgtggaaaggccttctctaggccctggctgcttcagggccacgtccgcacccacactggtgagaagccattctcctgctcccactgcaaccgtgcttttgctgaccgctccaacctgcgtgcccacctccaaacccactcggatgtgaagagataccagtgccaggcctgtgcccgaaccttctcccgcatgtccttgctccacaagcaccaagagtctggctgctccggaggccctcgctgagctgccttctgcggggcttgccttctggccatgcccttcttctctcccttgcacctgtacctcttggtctttgaataaagcctgagtaggaactgggcctcatgggccttccgctcactgcccgctttccag

SEQ ID NO: 30 - DNA, 732 bp, Artificial Sequence
  Mesp1
atggcccagcccctgtgcgagccgcgctccgagtcctggatcctgagtcccgctggtcggcagccaccgatgccttccgatgggaacagcgtctgctccccagcctggtcctcggacccgtgggacggtgcccaggccagcagccctgcaccaccctgcgcccgcccggcccggcgtgctgggaccccgggtaggcgcgggacgcacggtagccgcctgggtagcggacagcggcagagcgccagcgagcgggagaagctacgtatgcgcacactcgcccgcgcgctgcacgagctgcgccgcttcttgccgccatccgtggcaccaaccggccagaacctgaccaagatcgagacgctgcgcctggccatccgctacattggccacctgtcggctgtgctgggactcagcgaggacaacctccggcgacagcggcacgcggtgtcacctcgaggctgcccgctgtgccccgacagcgacctggcgcagtcgcagtcgctcggtccccgtttaagcccggccgtctgcagcggggtgtcgtggggatccccgcctgcctaccctagaccccgagtcgccgcagaatcgtgggacccatcgttcctgtacgcagaaacagcatcccaggaaaggcaggaaatggagcccagtccctcatctccgctcttcagcagcgacatgctggctcttctagaaacctggacgccgccgcaggagtggccgcctgcctga

SEQ ID NO: 31 - DNA, 306 bp, Artificial Sequence
  Sumo1
atgtctgaccaggaggcaaaaccttcaactgaggacttaggcgataagaaggaaggagaatacattaaactcaaagttattggacaggatagcagtgagatacatttcaaagtgaaaatgacaacacatctcaagaaactcaaagaatcatactgtcaaagacagggagttccaatgaattcactcaggtttctctttgaaggtcagagaattgctgataatcatactccgaaagaactgggaatggaggaagaagatgtgattgaagtttatcaggaacaaacggggggtcactcgacggtttag

SEQ ID NO: 32 - DNA, 957 bp, Artificial Sequence
  NKX2.5
atgttccccagccctgcgctcacacccacgcctttctcagtcaaagacatcctgaacctggagcagcagcagcgtagcctggcgtctggggacctgtctgcgcgcctcgaggccaccctggcccctgcctcctgcatgctggccgccttcaagcccgaggcctactctggccccgaggcggcagcgtccggcctggcagagctgcgcgcggagatgggccccgcgccttcgccccccaagtgctctcctgctttcccagccgcccccacattttacccgggagcctacggtgaccctgacccagccaaagaccctcgggcggataaaaaagagctgtgcgcgctgcagaaggcagtggagctggacaaagccgagacggatggcgccgagagaccacgcgcacggcggcgacggaagccacgcgtgctcttctcgcaggcgcaggtctacgagctggagcggcgcttcaagcaacagcggtacctgtcggcgccagagcgcgaccagctggccagcgtgctgaagctcacgtccacgcaggtcaagatctggttccagaaccgtcgctacaagtgcaagcgacagcggcaggaccagactctggagcttctggggccgccgccgccgcccgcgcgcaggatcgcggtgcccgtgctggtgcgcgacgggaagccctgcctgggggaccccgcggcctacgctcccgcctacggcgtgggtctcaatgcctatggctacaacgcctacccctaccccagctacggcggcgcggcctgcagtcccggctacagctgcgccgcctaccccgctgcgccccccgccgcgcagccccccgccgcctccgccaacagcaacttcgtgaactttggcgtcggggacttgaacaccgtgcagagtcccgggatgccgcagggcaattcgggcgtctccacgctgcacggcatccgagcctggtag

SEQ ID NO: 33 - DNA, 1311 bp, Artificial Sequence
  Tbx6
atgtaccatccacgagagttgtacccctccctggggactggctaccgtctgggacacccccagcctggggcagactccaccttcccacctgccctgacagagggttaccgctaccctgatttggatacttctaaactggattgcttcctctctgggatcgaggcagctccccacactctggctgcagccgctcctttgccccttctcccatctgctctgggccccgagacagcaccgccacccccagaggcccttcactcgcttcctggggtcagcctgagcttggagaaccaggaactgtggaaggaattcagcgctgtggggacagagatgatcatcaccaaggctggcaggcgcatgttccctgcttgccgagtatcagtcactggcctggacccagaggcccgctacttgtttcttctggatgtggttccagtggatggggcccgataccgctggcagggccagcactgggagccaagtggcaaggctgaaccccgcctacccgaccgtgtctacattcaccctgactctcctgccactggtgcccactggatgcggcagcccgtatccttccatcgtgttaagctcaccaacagcacactggacccccatggccacctgatcttgcactcgatgcacaagtaccagcctcgcatccacctggtgagagccacccaactatgcagccaacactgggggggtgtggcctccttccgatttcctgagaccacattcatctctgtgacagcctaccagaaccctaggatcacacagctgaagatcgcagccaatccctttgccaaaggtttccgagaaaatggcagaaactgtaagagggagcgggatgcccgtgtgaagaggaaacttcggggcccagagccagtggccacagaggcctgtgggagtggggatacaccagggggtccctgtgactccaccctgggtggggacattcgggactcagatccagagcaggccccaaccccccaggaagctgcttctgcctcagctcctccatgtgggggccccagtgctgaggcctaccttctacaccctgccgcttttcatggcgcccccagtcacctaccagccaggacccccagcttcgctgaggctccagaccctgggcgcccagccccctactcagctgcatttctggacctacagcctggaccagggggctctgcctatcaggcagctccatctgtaccatcctttgccccacacttcatccaagggggtcccttccctctaccgtacccaggacctggaggttatctggacatgggatccaagccaatgtactga

SEQ ID NO: 34 - DNA, 381 bp, Artificial Sequence
  Ad52E4
atggctgatcaacatatatatgtgcatttgctgggacgtcgggcctttttgccccagcaacagggttattctaatatgtatgttcttttttcaccggaggattttgtgcttgctcccagagggattattttgctgtctttgcagctgtcgttggatattcccactgggtatctgggacgttttttttctgtggcggatatgaatgtgaggggggtattgctgtgtgctcaggagatccaaccgagtacatggtgggaagtttctgttgttctttttaatcattcggatgaattttaccgcggttcccggggacagccggttgcttgcctgctgctggagcgtgtcatatatcccaccgttcgccaagcttctttagtttaa

SEQ ID NO: 35 - DNA, 3210 bp, Artificial Sequence
  Human TERT
atgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactga

SEQ ID NO: 36 - DNA, 1776 bp, Artificial Sequence
  Pkm2
atgactcaacctttcaactttgcggcttttgagaaaattttcttccttggctcttcagggcaaagcatactccactctgagcgatctttccaagtgaggccaccaagccacactgagtcaacaaaacagcaatatgccatcagaagcccagacccagagaaccaaaggacttcaggaaccatgccgaagccacacagtgaagcagggactgccttcattcagacccagcagctccatgcagccatggctgacaccttcctggaacacatgtgccgcctggacattgactctgcccccatcacggcccgcaacactggcatcatttgtaccattgggcctgcttcccgatctgtggagatgctgaaggagatgattaagtctggaatgaatgtggctcggctgaatttctctcatggaacccatgagtaccatgcagagaccatcaagaatgtccgtgaagccacagaaagctttgcatctgatcccattctctaccgtcctgttgcggtggctctggatacaaagggacctgagatccggactggactcatcaagggcagcggcaccgctgaggtggagctgaagaagggagccactctgaagatcaccctggacaacgcttacatggagaagtgtgacgagaacatcctgtggctggactacaagaacatctgcaaggtggtggaggtgggcagcaagatctacgtggacgatgggctcatctcactgcaggtgaaggagaaaggcgctgacttcctggtgacggaggtggagaatggtggctccttgggcagcaagaagggcgtgaacctgccgggcgctgctgtggatctccccgctgtgtcggaaaaggacatccaggacctgaagtttggggtggagcaggatgtggacatggtgtttgcatctttcatccgcaaggcagccgacgtgcatgaagtcaggaaggtgctgggagagaagggcaagaacatcaagatcatcagcaaaatcgagaaccatgaaggcgtccgcaggtttgatgagatcttggaggccagtgatgggatcatggtggctcgtggtgacctgggcattgagattcctgcagagaaggtcttcctggctcagaagatgatgatcgggcgatgcaaccgagctgggaagcctgtcatctgtgccacacagatgctggagagcatgatcaagaagccacgccccacccgtgctgaaggcagtgatgtggccaatgcagtcctggatggagcagactgcatcatgctgtctggagaaacagccaagggggactaccctctggaggctgttcgcatgcagcacctgatagctcgggaggctgaggcagccatgttccaccgtctgctgtttgaagagcttgtgcgagcctccagtcactccacagacctcatggaggccatggccatgggcagcgtggaggcctcttataagtgtttagcagcagctttgatagttctcacggagtctggcaggagtgctcaccaagtggccaggtaccgccctcgggctcctatcattgccgtgactcgaaatccccagactgctcgccaggcccatctgtaccgtggcatcttccctgtgctgtgtaaggatgccgtgctgaatgcctgggctgaggatgtcgaccttcgtgtaaacttggccatggatgttggcaaggcccgaggcttcttcaagaagggagatgtggtcattgtgctgaccgggtggcgccctggctctggattcaccaacaccatgcgtgtagtgcctgtaccttga

SEQ ID NO: 37 - protein, 442 aa, Artificial Sequence
  Mouse GATA4
MYQSLAMAANHGPPPGAYEAGGPGAFMHSAGAASSPVYVPTPRVPSSVLGLSYLQGGGSAAAAGTTSGGSSGAGPSGAGPGTQQGSPGWSQAGAEGAAYTPPPVSPRFSFPGTTGSLAAAAAAAAAREAAAYGSGGGAAGAGLAGREQYGRPGFAGSYSSPYPAYMADVGASWAAAAAASAGPFDSPVLHSLPGRANPGRHPNLVDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAGPAGETLPPSSGASSGNSSNATSSSSSSEEMRPIKTEPGLSSHYGHSSSMSQTFSTVSGHGPSIHPVLSALKLSPQGYASPVTQTSQASSKQDSWNSLVLADSHGDIITA

SEQ ID NO: 38 - protein, 466 aa, Artificial Sequence
  Mouse Mef2c
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPSFEMPVTIPVSSHNSLVYSNPVSTLGNPNLLPLAHPSLQRNSMSPGVTHRPPSAGNTGGLMGGDLTSGAGTSAGNGYGNPRNSPGLLVSPGNLNKNIQAKSPPPMNLGMNNRKPDLRVLIPPGSKNTMPSVNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLSIKSEPVSPPRDRTTTPSRYPQHTTRHEAGRSPVDSLSSCSSSYDGSDREDHRNEFHSPIGLTRPSPDERESPSVKRMRLSEGWAT

SEQ ID NO: 39 - protein, 518 aa, Artificial Sequence
  Mouse Tbx5
MADTDEGFGLARTPLEPDSKDRSCDSKPESALGAPSKSPSSPQAAFTQQGMEGIKVFLHERELWLKFHEVGTEMIITKAGRRMFPSYKVKVTGLNPKTKYILLMDIVPADDHRYKFADNKWSVTGKAEPAMPGRLYVHPDSPATGAHWMRQLVSFQKLKLTNNHLDPFGHIILNSMHKYQPRLHIVKADENNGFGSKNTAFCTHVFPETAFIAVTSYQNHKITQLKIENNPFAKGFRGSDDLELHRMSRMQSKEYPVVPRSTVRHKVTSNHSPFSSETRALSTSSNLGSQYQCENGVSGPSQDLLPPPNPYPLAQEHSQIYHCTKRKDEECSSTEHPYKKPYMETSPSEEDTFYRSGYPQQQGLSTSYRTESAQRQACMYASSAPPSEPVPSLEDISCNTWPSMPSYSSCTVTTVQPMDRLPYQHFSAHFTSGPLVPRLAGMANHGSPQLGEGMFQHQTSVAHQPVVRQCGPQTGLQSPGGLQPPEFLYTHGVPRTLSPHQYHSVHGVGMVPEWSENS

SEQ ID NO: 40 - protein, 217 aa, Artificial Sequence
  Mouse Hand2
MSLVGGFPHHPVVHHEGYPFAAAAAAAAAAAASRCSHEENPYFHGWLIGHPEMSPPDYSMALSYSPEYASGAAGLDHSHYGGVPPGAGPPGLGGPRPVKRRGTANRKERRRTQSINSAFAELRECIPNVPADTKLSKIKTLRLATSYIAYLMDLLAKDDQNGEAEAFKAEIKKTDVKEEKRKKELNEILKSTVSSNDKKTKGRTGWPQHVWALELKQ

SEQ ID NO: 41 - protein, 395 aa, Artificial Sequence
  ASAH1
MPGRSCVALVLLAAAVSCAVAQHAPPWTEDCRKSTYPPSGPTYRGAVPWYTINLDLPPYKRWHELMLDKAPVLKVIVNSLKNMINTFVPSGKIMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDVMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 42 - protein, 202 aa, Artificial Sequence
  dnTGFB
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVGSMDYKDDDDK

SEQ ID NO: 43 - protein, 225 aa, Artificial Sequence
  dnWnt8a
  misc_feature (223)..(224): Xaa can be any naturally occurring amino acid
MQNTTLFILATLLIFCPFFTASAWSVNNFLMTGPKAYLTYSASVAVGAQNGIEECKYQFAWERWNCPESTLQLATHNGLRSATRETSFVHAISSAGVMYTLTRNCSMGDFDNCGCDDSRNGRIGGRGWVWGGCSDNAEFGERISKLFVDGLETGQDARALMNLHNNEAGRLAVKETMKRTCKCHGISGSCSIQTCWLQLAEFRDIGNPLKIKHDQALKLEMDXXK

SEQ ID NO: 44 - protein, 443 aa, Artificial Sequence
  Human GATA4
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLVDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 45 - protein, 463 aa, Artificial Sequence
  Human Mef2c
MGRKKIQITRIMDERNRQVTFTKRKFGLMKKAYELSVLCDCEIALIIFNSTNKLFQYASTDMDKVLLKYTEYNEPHESRTNSDIVEALNKKENKGCESPDPDSSYALTPRTEEKYKKINEEFDNMIKSHKIPAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 46 - protein, 518 aa, Artificial Sequence
  Human Tbx5
MADADEGFGLAHTPLEPDAKDLPCDSKPESALGAPSKSPSSPQAAFTQQGMEGIKVFLHERELWLKFHEVGTEMIITKAGRRMFPSYKVKVTGLNPKTKYILLMDIVPADDHRYKFADNKWSVTGKAEPAMPGRLYVHPDSPATGAHWMRQLVSFQKLKLTNNHLDPFGHIILNSMHKYQPRLHIVKADENNGFGSKNTAFCTHVFPETAFIAVTSYQNHKITQLKIENNPFAKGFRGSDDMELHRMSRMQSKEYPVVPRSTVRQKVASNHSPFSSESRALSTSSNLGSQYQCENGVSGPSQDLLPPPNPYPLPQEHSQIYHCTKRKEEECSTTDHPYKKPYMETSPSEEDSFYRSSYPQQQGLGASYRTESAQRQACMYASSAPPSEPVPSLEDISCNTWPSMPSYSSCTVTTVQPMDRLPYQHFSAHFTSGPLVPRLAGMANHGSPQLGEGMFQHQTSVAHQPVVRQCGPQTGLQSPGTLQPPEFLYSHGVPRTLSPHQYHSVHGVGMVPEWSDNS

SEQ ID NO: 47 - protein, 217 aa, Artificial Sequence; identical to SEQ ID NO: 40
  Human Hand2

SEQ ID NO: 48 - protein, 986 aa, Artificial Sequence
  Human Myocardin
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 49 - DNA, 21 bp, Artificial Sequence
  primer
aggtcggtgtgaacggatttg

SEQ ID NO: 50 - DNA, 23 bp, Artificial Sequence
  primer
tgtagaccatgtagttgaggtca

SEQ ID NO: 51 - DNA, 21 bp, Artificial Sequence
  primer
taacgaacgagactctggcat

SEQ ID NO: 52 - DNA, 22 bp, Artificial Sequence
  primer
cggacatctaagggcatcacag

SEQ ID NO: 53 - DNA, 20 bp, Artificial Sequence
  primer
ggtgtgcggataaaagacgg

SEQ ID NO: 54 - DNA, 20 bp, Artificial Sequence
  primer
tggtccacctaagagggtgt

SEQ ID NO: 55 - DNA, 20 bp, Artificial Sequence
  primer
ctgagacagaggaggccaac

SEQ ID NO: 56 - DNA, 20 bp, Artificial Sequence
  primer
ttccgctctgtcttctggat

SEQ ID NO: 57 - DNA, 20 bp, Artificial Sequence
  primer
ctgtgcaggctgctgtaacg

SEQ ID NO: 58 - DNA, 20 bp, Artificial Sequence
  primer
gttcccgaaaccctgaggag

SEQ ID NO: 59 - DNA, 18 bp, Artificial Sequence
  primer
cccctgtgtcccagtttg

SEQ ID NO: 60 - DNA, 19 bp, Artificial Sequence
  primer
cgtctatccatggcaccac

SEQ ID NO: 61 - DNA, 27 bp, Artificial Sequence
  primer
cagacaagttcattcaattattagacg

SEQ ID NO: 62 - DNA, 21 bp, Artificial Sequence
  primer
catgtcttgttagctgcctga

SEQ ID NO: 63 - DNA, 25 bp, Artificial Sequence
  primer
gcaactttctatgacactgaaacac

SEQ ID NO: 64 - DNA, 20 bp, Artificial Sequence
  primer
tctctctagggctgcattgg

SEQ ID NO: 65 - DNA, 20 bp, Artificial Sequence
  primer
cacagatggccttgatgttg

SEQ ID NO: 66 - DNA, 20 bp, Artificial Sequence
  primer
ctctggctcagcatgactcc

SEQ ID NO: 67 - DNA, 19 bp, Artificial Sequence
  primer
gggggaggttggacagtaa

SEQ ID NO: 68 - DNA, 19 bp, Artificial Sequence
  primer
catcagctcaatcctcagc

SEQ ID NO: 69 - DNA, 19 bp, Artificial Sequence
  primer
caagcagtgcctatccaga

SEQ ID NO: 70 - DNA, 20 bp, Artificial Sequence
  primer
aagcccaggaatgaagtcca

SEQ ID NO: 71 - DNA, 20 bp, Artificial Sequence
  primer
ttcaaccatgctcctggata

SEQ ID NO: 72 - DNA, 20 bp, Artificial Sequence
  primer
gaccattcctcattgcacac

SEQ ID NO: 73 - DNA, 21 bp, Artificial Sequence
  primer
gctgcgcttgcagagattaaa

SEQ ID NO: 74 - DNA, 20 bp, Artificial Sequence
  primer
ttgctgtactgtgtgtccag

SEQ ID NO: 75 - DNA, 25 bp, Artificial Sequence
  primer
gtaaatgggattccaacacgaacaa

SEQ ID NO: 76 - DNA, 24 bp, Artificial Sequence
  primer
tgtcgtgcagtaagaacccaactc

SEQ ID NO: 77 - DNA, 24 bp, Artificial Sequence
  primer
gccattatgcagctgctttggagc

SEQ ID NO: 78 - DNA, 24 bp, Artificial Sequence
  primer
tgtttttcttgggcttaatcacct

SEQ ID NO: 79 - DNA, 23 bp, Artificial Sequence
  primer
gttgaccctaccatgttcccttg

SEQ ID NO: 80 - DNA, 20 bp, Artificial Sequence
  primer
gccagcagcatctatgggac

SEQ ID NO: 81 - DNA, 24 bp, Artificial Sequence
  primer
gaaacactcttctgtaacacactt

SEQ ID NO: 82 - DNA, 22 bp, Artificial Sequence
  primer
gtcaaactacaactccaagcag

SEQ ID NO: 83 - DNA, 21 bp, Artificial Sequence
  primer
gcaactccgtccgggcgagga

SEQ ID NO: 84 - DNA, 21 bp, Artificial Sequence
  primer
gaagatgactgtggtcccggg

SEQ ID NO: 85 - DNA, 22 bp, Artificial Sequence
  primer
aaacccgatatggctgagattg

SEQ ID NO: 86 - DNA, 20 bp, Artificial Sequence
  primer
gcctgcttgcttctcctgtt

SEQ ID NO: 87 - DNA, 23 bp, Artificial Sequence
  primer
cccctgcccattcggaggaagag

SEQ ID NO: 88 - DNA, 23 bp, Artificial Sequence
  primer
ttggccaccttgacgctgcggtg

SEQ ID NO: 89 - DNA, 22 bp, Artificial Sequence
  primer
ttgccttgctgctctacctcca

SEQ ID NO: 90 - DNA, 22 bp, Artificial Sequence
  primer
gatggcagtagctgcgctgata

SEQ ID NO: 91 - DNA, 21 bp, Artificial Sequence
  primer
tgcctgccgtgtgaaccatgt

SEQ ID NO: 92 - DNA, 24 bp, Artificial Sequence
  primer
tgcggcatcttcaaacctccatga